SEQUENCE LISTING (73 sequences)

SEQ ID NO: 1 - DNA, 17 bp, Artificial Sequence
  Primer
gacctgatgcaccggcg

SEQ ID NO: 2 - DNA, 19 bp, Artificial Sequence
  Primer
atgtataaatgcccttctc

SEQ ID NO: 3 - DNA, 18 bp, Artificial Sequence
  Primer
ttgcgcaacttgggcctg

SEQ ID NO: 4 - DNA, 17 bp, Artificial Sequence
  Primer
acccaccggagtggttg

SEQ ID NO: 5 - DNA, 18 bp, Artificial Sequence
  Primer
gccgccttcgccaaccac

SEQ ID NO: 6 - DNA, 19 bp, Artificial Sequence
  Primer
acggctttgtccaagagac

SEQ ID NO: 7 - DNA, 18 bp, Artificial Sequence
  Primer
gtcctcgtcctcctgcat

SEQ ID NO: 8 - DNA, 18 bp, Artificial Sequence
  Primer
tcagtcaggtctcggggg

SEQ ID NO: 9 - DNA, 19 bp, Artificial Sequence
  Primer
ccgttctgtcccgagtatg

SEQ ID NO: 10 - DNA, 18 bp, Artificial Sequence
  Primer
ggtcaccatcccggcaga

SEQ ID NO: 11 - DNA, 19 bp, Artificial Sequence
  Primer
cgtgagcccatctgggaaa

SEQ ID NO: 12 - DNA, 19 bp, Artificial Sequence
  Primer
gaggtcaggcttacaggat

SEQ ID NO: 13 - DNA, 21 bp, Artificial Sequence
  Primer
accatggtgtctttgctttcc

SEQ ID NO: 14 - DNA, 17 bp, Artificial Sequence
  Primer
gtgagcaggggacccga

SEQ ID NO: 15 - DNA, 18 bp, Artificial Sequence
  Primer
gtgtccagaggagcccat

SEQ ID NO: 16 - DNA, 17 bp, Artificial Sequence
  Primer
gtggcagggggcttggt

SEQ ID NO: 17 - DNA, 18 bp, Artificial Sequence
  Primer
gtgttcctggcgcgctat

SEQ ID NO: 18 - DNA, 17 bp, Artificial Sequence
  Primer
gtaaggggtcgccttcc

SEQ ID NO: 19 - DNA, 19 bp, Artificial Sequence
  Primer
aggccttcctggcagagat

SEQ ID NO: 20 - DNA, 18 bp, Artificial Sequence
  Primer
tcattcctcctgggacgc

SEQ ID NO: 21 - DNA, 19 bp, Artificial Sequence
  Primer
aggatcctgtaagcctgac

SEQ ID NO: 22 - DNA, 20 bp, Artificial Sequence
  Primer
atgaatcacggcagtggtgt

SEQ ID NO: 23 - DNA, 19 bp, Artificial Sequence
  Primer
agggccactttgtgaagcc

SEQ ID NO: 24 - DNA, 21 bp, Artificial Sequence
  Primer
caggaaagcaaagacaccatg

SEQ ID NO: 25 - DNA, 18 bp, Artificial Sequence
  Primer
gcgtttctccctcatgac

SEQ ID NO: 26 - DNA, 20 bp, Artificial Sequence
  Primer
ggtcagtgatatggagtagg

SEQ ID NO: 27 - DNA, 18 bp, Artificial Sequence
  Primer
ctgcatgcaagacaggag

SEQ ID NO: 28 - DNA, 23 bp, Artificial Sequence
  Primer
ccttgggaatgagatagtttctg

SEQ ID NO: 29 - DNA, 23 bp, Artificial Sequence
  Primer
cagatatgcaataattttcccac

SEQ ID NO: 30 - DNA, 26 bp, Artificial Sequence
  Primer
gcaaggtttttaagtaatttgttatg

SEQ ID NO: 31 - DNA, 24 bp, Artificial Sequence
  Primer
ccattattttccagaaacgtttcg

SEQ ID NO: 32 - DNA, 23 bp, Artificial Sequence
  Primer
ggatttcccagaaaaaaagactg

SEQ ID NO: 33 - DNA, 23 bp, Artificial Sequence
  Primer
aacaaagttttagcaaacgattt

SEQ ID NO: 34 - DNA, 17 bp, Artificial Sequence
  Primer
atgcccatcgtggcgca

SEQ ID NO: 35 - DNA, 23 bp, Artificial Sequence
  Primer
ggctcttatctacataggttgtt

SEQ ID NO: 36 - DNA, 20 bp, Artificial Sequence
  Primer
ctatgggagtctcccctatt

SEQ ID NO: 37 - DNA, 17 bp, Artificial Sequence
  Probe
  misc_feature (1)..(1): 56-FAM
  misc_feature (17)..(17): 3BHQ_1
ctggtgggtagcgtgca

SEQ ID NO: 38 - DNA, 19 bp, Artificial Sequence
  Probe
  misc_feature (1)..(1): 5HEX
  misc_feature (19)..(19): 3IABkFQ
cctggtgagtagcgtgcag

SEQ ID NO: 39 - DNA, 20 bp, Artificial Sequence
  Probe
  misc_feature (1)..(1): 56-FAM
  misc_feature (20)..(20): 3IABkFQ
tcggtcacccactgctccag

SEQ ID NO: 40 - DNA, 19 bp, Artificial Sequence
  Probe
  misc_feature (1)..(1): 5HEX
  misc_feature (19)..(19): 3IABkFQ
tcggtcacccctgctccag

SEQ ID NO: 41 - DNA, 18 bp, Artificial Sequence
  Probe
  misc_feature (1)..(1): 56-FAM
  misc_feature (18)..(18): 3IABkFQ
acccccaggacgcccctt

SEQ ID NO: 42 - DNA, 19 bp, Artificial Sequence
  Probe
  misc_feature (1)..(1): 5HEX
  misc_feature (19)..(19): 3IABkFQ
acccccaagacgccccttt

SEQ ID NO: 43 - DNA, 21 bp, Artificial Sequence
  Probe
  misc_feature (1)..(1): 56-FAM
  misc_feature (21)..(21): 3BHQ_1
tcccaggtcatcctgtgctca

SEQ ID NO: 44 - DNA, 18 bp, Artificial Sequence
  Probe
  misc_feature (1)..(1): 5HEX
  misc_feature (18)..(18): 3IABkFQ
caggtcatccgtgctcag

SEQ ID NO: 45 - DNA, 20 bp, Artificial Sequence
  Probe
  misc_feature (1)..(1): 56-FAM
  misc_feature (20)..(20): 3BHQ_1
agccaccactatgcgcaggt

SEQ ID NO: 46 - DNA, 20 bp, Artificial Sequence
  Probe
  misc_feature (1)..(1): 5HEX
  misc_feature (20)..(20): 3IABkFQ
agccaccactatgcacaggt

SEQ ID NO: 47 - DNA, 19 bp, Artificial Sequence
  Probe
  misc_feature (1)..(1): 56-FAM
  misc_feature (19)..(19): 3BHQ_1
agggaggaagggtacaggc

SEQ ID NO: 48 - DNA, 19 bp, Artificial Sequence
  Probe
  misc_feature (1)..(1): 5HEX
  misc_feature (19)..(19): 3IABkFQ
agggagaaagggtacaggc

SEQ ID NO: 49 - DNA, 20 bp, Artificial Sequence
  Probe
  misc_feature (1)..(1): 56-FAM
  misc_feature (20)..(20): 3BHQ_1
tggtgagcccatccccctat

SEQ ID NO: 50 - DNA, 20 bp, Artificial Sequence
  Probe
  misc_feature (1)..(1): 5HEX
  misc_feature (20)..(20): 3IABkFQ
tggtgaccccatccccctat

SEQ ID NO: 51 - DNA, 20 bp, Artificial Sequence
  Probe
  misc_feature (1)..(1): 56-FAM
  misc_feature (20)..(20): 3BHQ_1
tggtgcccctggccgtgata

SEQ ID NO: 52 - DNA, 20 bp, Artificial Sequence
  Probe
  misc_feature (1)..(1): 5HEX
  misc_feature (20)..(20): 3IABkFQ
tggtgcccctggccatgata

SEQ ID NO: 53 - DNA, 19 bp, Artificial Sequence
  Probe
  misc_feature (1)..(1): 56-FAM
  misc_feature (19)..(19): 3IABkFQ
tcgccaaccactccggtgg

SEQ ID NO: 54 - DNA, 19 bp, Artificial Sequence
  Probe
  misc_feature (1)..(1): 5HEX
  misc_feature (19)..(19): 3IABkFQ
tcgccaaccactccagtgg

SEQ ID NO: 55 - DNA, 19 bp, Artificial Sequence
  Probe
  misc_feature (1)..(1): 5Cy5
  misc_feature (19)..(19): 3IAbRQSp
tcgccaaccactcctgtgg

SEQ ID NO: 56 - DNA, 22 bp, Artificial Sequence
  Probe
  misc_feature (1)..(1): 56-FAM
  misc_feature (22)..(22): 3IABkFQ
agagatggagaaggtgagagtg

SEQ ID NO: 57 - DNA, 19 bp, Artificial Sequence
  Probe
  misc_feature (1)..(1): 5HEX
  misc_feature (19)..(19): 3IABkFQ
agagatggaggtgagagtg

SEQ ID NO: 58 - DNA, 21 bp, Artificial Sequence
  Probe
  misc_feature (1)..(1): 56-FAM
  misc_feature (21)..(21): 3IABkFQ
atcgacgacgtgatagggcag

SEQ ID NO: 59 - DNA, 21 bp, Artificial Sequence
  Probe
  misc_feature (1)..(1): 5HEX
  misc_feature (21)..(21): 3IABkFQ
atcgacgacatgatagggcag

SEQ ID NO: 60 - DNA, 18 bp, Artificial Sequence
  Probe
  misc_feature (1)..(1): 56-FAM
  misc_feature (18)..(18): 3BHQ_1
cacaggccgccgtgcatg

SEQ ID NO: 61 - DNA, 19 bp, Artificial Sequence
  Probe
  misc_feature (1)..(1): 5HEX
  misc_feature (19)..(19): 3IABkFQ
ccacaggccaccgtgcatg

SEQ ID NO: 62 - DNA, 22 bp, Artificial Sequence
  Probe
  misc_feature (1)..(1): 56-FAM
  misc_feature (22)..(22): 3BHQ_1
cattgaggaccgtgttcaagag

SEQ ID NO: 63 - DNA, 22 bp, Artificial Sequence
  Probe
  misc_feature (1)..(1): 5HEX
  misc_feature (22)..(22): 3BHQ_1
cattgaggactgtgttcaagag

SEQ ID NO: 64 - DNA, 21 bp, Artificial Sequence
  Probe
  misc_feature (1)..(1): 56-FAM
  misc_feature (21)..(21): 3BHQ_1
cgaggtccagagatacattga

SEQ ID NO: 65 - DNA, 21 bp, Artificial Sequence
  Probe
  misc_feature (1)..(1): 5HEX
  misc_feature (21)..(21): 3IABkFQ
cgaggtccagagataccttga

SEQ ID NO: 66 - DNA, 22 bp, Artificial Sequence
  Probe
  misc_feature (1)..(1): 56-FAM
  misc_feature (22)..(22): 3BHQ_1
tcattgattatttcccgggaac

SEQ ID NO: 67 - DNA, 22 bp, Artificial Sequence
  Probe
  misc_feature (1)..(1): 5HEX
  misc_feature (22)..(22): 3IABkFQ
tcattgattatttcccaggaac

SEQ ID NO: 68 - DNA, 21 bp, Artificial Sequence
  Probe
  misc_feature (1)..(1): 56-FAM
  misc_feature (21)..(21): 3IABkFQ
taagcaccccctggatccagg

SEQ ID NO: 69 - DNA, 21 bp, Artificial Sequence
  Probe
  misc_feature (1)..(1): 5HEX
  misc_feature (21)..(21): 3IABkFQ
taagcaccccctgaatccagg

SEQ ID NO: 70 - DNA, 20 bp, Artificial Sequence
  Probe
  misc_feature (1)..(1): 56-FAM
  misc_feature (20)..(20): 3BHQ_1
tcttctgttctcaaagcatc

SEQ ID NO: 71 - DNA, 20 bp, Artificial Sequence
  Probe
  misc_feature (1)..(1): 5HEX
  misc_feature (20)..(20): 3IABkFQ
tgtcttctgttctcaaagta

SEQ ID NO: 72 - DNA, 23 bp, Artificial Sequence
  Probe
  misc_feature (1)..(1): 56-FAM
  misc_feature (23)..(23): 3BHQ_1
tatgtgttcatgggtaatatgct

SEQ ID NO: 73 - DNA, 22 bp, Artificial Sequence
  Probe
  misc_feature (1)..(1): 5HEX
  misc_feature (22)..(22): 3IABkFQ
atatgcgttcatgggtaatatg

(19) United States
(12) Patent Application Publication
(10) Pub. No.: US 2024/0301501 A1
(43) Pub. Date: Sep. 12, 2024

(54) A METHOD FOR ASSESSING THE POTENTIAL EFFECT OF THERAPEUTICS ON AN INDIVIDUAL

(71) Applicants: AGENCY FOR SCIENCE TECHNOLOGY AND RESEARCH, Singapore (SG); NALAGENETICS PTE. LTD., Singapore (SG)

(72) Inventors: Alexander Lezhava, Genome (SG); Astrid Irwanto, Genome (SG); Kanika Jain, Genome (SG); Anar Sanjaykumar Kothary, Genome (SG); Jocelyn Tan, Genome (SG); Flona Ng, Genome (SG); Zhihao Tan, Genome (SG)

(21) Appl. No.: 18/281,523

(22) PCT Filed: Mar. 11, 2022
(86) PCT No.: PCT/SG2022/050127

§ 371 Date: Sep. 11, 2023

(51) Int. Cl. C12Q 1/6886

(52) U.S. Cl. CPC C12Q 1/6886; C12Q 2600/106; C12Q 2600/156

(57) ABSTRACT

The invention relates to a method for assessing and evaluating the potential effect of therapeutics on an individual. In particular, the invention uses real-time PCR-based pharmacogenomic assays in assessing such potential effects. In an aspect of the present invention, there is provided a method of assessing or evaluating a subject's likelihood of developing an adverse reaction in response to an administration of a therapeutic agent, or a method of assessing or evaluating a therapeutic agent's efficacy on a subject, the method comprising determining in a single real-time polymerase chain reaction run the presence of a variant in a set of genes consisting of CYP2D6, CYP2C9, CYP2C19 and SLCO1B1 in a sample obtained from the subject, wherein the presence of a variant on any one of the genes in the set of genes is indicative of a risk to an adverse reaction and/or a change in efficacy to the therapeutic agent.

The present application claims priority to Singapore patent application number 10202102511P filed on 11 Mar. 2021 which is incorporated by reference herein in its entirety.

The invention relates to a method for assessing and evaluating the potential effect of therapeutics on an individual. In particular, the invention uses real-time PCR-based pharmacogenomic assays in assessing such potential effects.

Adverse drug reactions are a major clinical problem. Although drug eruptions may be mild to moderate, such as maculopapular rash, erythema multiforme, urticaria, and fixed drug eruption, more severe reactions are life threatening and frequently result in death. In addition, hypersensitivity reactions to certain therapeutics can occur. Common symptoms may include fever, rash, gastrointestinal reactions, severe fatigue, and respiratory symptoms.

Recent developments of pharmacogenomics have implied that the susceptibility to drug reactions and hypersensitivity may be associated with genetic variants.

Pharmacogenetics is the study of the role of inheritance in individual variation in response to drugs, nutrients and other xenobiotics, and in this post-genomic era, pharmacogenetics has evolved into pharmacogenomics. Drug response phenotypes that are influenced by inheritance can vary from potentially life-threatening adverse reactions at one of the spectrum to lack of therapeutic efficacy at the other. The ability to determine whether and how a subject will respond to a particular drug can assist medical professionals in determining whether the drug should be administered to the subject, and at what dose.

A major challenge facing this component of individualized medicine is that current pharmacogenomics testing solutions using qPCR platform are not scalable due to different cycling conditions and preparations that require separate qPCR runs. This limits the use of pharmacogenomics testing to purely reactive testing. However, as implementation of genetic testing is increasingly growing into screening and pre-emptive uses in primary care settings, a new pharmacogenomics test needs to be developed that aims to provide a more efficient test that combines multiple variants to be tested together in one condition, especially to be prescribed in outpatient settings or through General Practitioners.

The listing or discussion of an apparently prior-published document in this specification should not necessarily be taken as an acknowledgement that the document is part of the state of the art or is common general knowledge.

Any document referred to herein is hereby incorporated by reference in its entirety.

In an aspect of the present invention, there is provided a method of assessing or evaluating a subject's likelihood of developing an adverse reaction in response to an administration of a therapeutic agent, or a method of assessing or evaluating a therapeutic agent's efficacy on a subject, the method comprising determining in a single real-time polymerase chain reaction run the presence of a variant in a set of genes consisting of CYP2D6, CYP2C9, CYP2C19 and SLCO1B1 in a sample obtained from the subject, wherein the presence of a variant on any one of the genes in the set of genes is indicative of a risk to an adverse reaction and/or a change in efficacy to the therapeutic agent.

By “risk to an adverse reaction”, it is meant to include any possibility of an adverse drug reaction (ADR) caused by the administration of the therapeutic agent. ADRs may occur following a single dose or prolonged administration of a drug or result from the combination of two or more drugs. For the avoidance of doubt, the term ADRs also include any “side effects” (particularly non-beneficial or detrimental side effects) of the therapeutic agent.

By “assessing or evaluating”, it is meant to include any determination of a subject's response to the administration of a therapeutic agent. By “response”, it is meant to include any adverse reaction and/or efficacy to said therapeutic agent. The method of assessing or evaluating also includes any form of pharmacogenomics profiling which refers to the determination of genetic factors present in a subject that are associated with diseases or medical conditions, particularly adverse reactions and efficacy to drugs. Typically, a panel of genetic factors is determined in pharmacogenomics profiling, and the factors may or may not be associated with the same disease, medical condition, or reaction to drug.

By “variant” in the relevant gene, it is meant to include any variation or alteration in the sequences of said gene, such that the sequence differs from what is found naturally or in most people. Similarly, a “non-variant” may include any sequence of the gene that may be considered “wild-type”, i.e. a sequence that is deemed normal or typical for said gene. As such, a “variant” of the gene means any one or more alteration(s), i.e. a substitution, insertion, and/or deletion, at one or more (several) positions, of the polynucleotide of the gene. A substitution may include a replacement of one or more nucleotide(s) occupying a position with one or more different nucleotide(s); a deletion means removal of one or more nucleotide(s) occupying a position; and an insertion means adding one or more, preferably 1-3 nucleotide(s) immediately adjacent to an nucleotide occupying a position. The variant may vary from the wild type gene by at least 1% pure, or e.g., at least 5%, at least 10%, at least 20%, at least 40%, at least 60%, at least 80%, and at least 90%. The term “variant” is also intended to include any markers or biomarkers.

In addition, the term “variant” may include “allelic variant” which means any of two or more alternative forms of a gene occupying the same chromosomal locus. The terms “allelic variants” and “alleles” are used interchangeably. Allelic variation arises naturally through mutation, and may result in polymorphism within populations. Gene mutations can be silent (no change in the encoded polypeptide) or may encode polypeptides having altered amino acid sequences. Alleles may comprise one or more variants.

By “adverse reaction”, it is meant to include any undesired and unintended effect of that therapeutic agent drug. In particular, an adverse reaction occurs at doses used for prophylaxis, diagnosis or therapy.

By “change in efficacy”, it is meant to include any change in the subject's response to the therapeutic agent, i.e. whether the therapeutic agent demonstrates a health benefit to the subject. Any change in efficacy can be determined by various methods such as measuring, monitoring or determining a particular parameter associated with a symptom of the disease which the therapeutic agent aims to treat. In various embodiments of the invention, the change refers to a scenario where the therapeutic agent provides less or no health benefit to the subject compared to known benefits which the therapeutic agent should otherwise provide. In other embodiments of the invention, the change in efficacy may also refer to a scenario where the therapeutic agent provides more health benefits to the subject compared to known benefits which the therapeutic agent is expected to provide.

In various embodiments, the presence of a variant is determined by providing a plurality of primer pairs and probes for amplifying a nucleic acid in the sample, wherein each primer pair amplifies a region of the nucleic acid associated with the genes or its variant, and detecting the presence or absence of a polymerase chain reaction product is indicative of the variant. The presence of a variant may be determined by detecting copy number variations (CNVs), insertions deletions (indels) or single nucleotide polymorphisms (SNPs) of the subject. In various embodiments, the step of determining the presence of the copy number variation further comprises providing a control having a human genomic DNA to determine the subject's CYP2D6 gene copy number variations.

The plurality of primer pairs comprises at least one primer pair for amplifying a conserved area of the gene. In addition, where the variant is a copy number variation, the step of determining the presence of the copy number variation further comprises an RNaseP as a housekeeping gene.

In various embodiments, the variant of the gene is any variant selected from the group consisting of rs1065852, rs5030655, rs3892097, rs35742686, rs16947, rs28371725, rs1135840, rs769258, rs5030865, rs5030656, rs59421388, rs267608319, exon 9 conversion (*36), deletion (*5), rs1799853, rs1057910, rs4244285, rs4986893, rs12248560 and rs4149056. Table 1 below shows the relevant genes of the invention and their associated variants.

TABLE 1

| Gene | Variants |
|---|---|
| CYP2D6 | rs1065852, rs5030655, rs3892097, rs35742686, rs16947, |
| | rs28371725, rs1135840, rs769258, rs5030865, rs5030656, |
| | rs59421388, rs267608319, exon 9 conversion (*36), deletion (*5) |
| CYP2C9 | rs1799853, rs1057910 |
| CYP2C19 | rs4244285, rs4986893, rs12248560 |
| SLCO1B1 | rs4149056 |

In various embodiments, the probes for targeting wild-type (or non-variant) genes are tagged with a FAM fluorophore at the 5′ end, and the probes for targeting variant genes are tagged with HEX or Cy5 fluorophore at the 5′ end. The probes for targeting the copy number variation of CYP2D6 are tagged with a FAM fluorophore at the 5′, and the probes for targeting the housekeeping gene are tagged with a VIC fluorophore at the 5′ end. In various embodiments, the ratio between primer pairs and FAM, HEX, Cy5 and VIC probes may be asymmetric.

In various embodiments, the probes have a 3′ modification of either a BHQ1 quencher, an IBFQ quencher, or an IBRQ quencher.

By “therapeutic agents”, it includes any drug or medication that is a compound or material that is administered to a patient for prophylactic, diagnostic or therapeutic purposes. In various embodiments, the therapeutic agents are selected based on the availability of scientific evidence, drug labels and/or clinical guidelines, and may include its derivatives. Non-limiting examples of therapeutic agents are set out in Table 2. In various embodiments, the therapeutic agent is any one selected from the list in Table 2.

TABLE 2

| | | | | |
|---|---|---|---|---|
| abiraterone | cobimetinib | fluoxetine/ | modafinil | rucaparib |
| | | olanzapine | | |
| acenocoumarol | codeine | flupenthixol | nebivolol | ruxolitinib |
| allopurinol | crizotinib | fluphenazine | nefazodone | sertraline |
| amiodarone | dabrafenib | flurbiprofen | nelfinavir | sildenafil |
| amitriptyline | darifenacin | fluvastatin | nortriptyline | simeprevir |
| amoxapine | dasabuvir/ | fluvoxamine | olanzapine | simvastatin |
| | ombitasvir/ | | | |
| | paritaprevir/ | | | |
| | ritonavir | | | |
| amphetamine | dasatinib | formoterol | ombitasvir/ | siponimod |
| | | | paritaprevir/ | |
| | | | ritonavir | |
| anastrozole | desipramine | galantamine | omeprazole | sofosbuvir/ |
| | | | | velpatasvir |
| arformoterol | desvenlafaxine | gefitinib | ondansetron | sotalol |
| aripiprazole | deutetrabenazine | glibenclamide | oxcarbazepine | sulfamethoxazole/ |
| | | | | trimethoprim |
| aripiprazole | dexlansoprazole | gliclazide | oxycodone | tamoxifen |
| lauroxil | | | | |
| atazanavir | dextromethorphan/ | glimepiride | palonosetron | tamsulosin |
| | quinidine | | | |
| atenolol | diazepam | haloperidol | pantoprazole | terbinafine |
| atomoxetine | disopyramide | ibrutinib | paroxetine | tetrabenazine |
| atorvastatin | donepezil | iloperidone | pazopanib | thioridazine |
| belinostat | doxepin | imatinib | perphenazine | ticagrelor |
| bisoprolol | dronabinol | imipramine | phenprocoumon | timolol |
| brexpiprazole | drospirenone/ | ivacaftor/ | phenytoin | tiotropium |
| | ethinyl estradiol | lumacaftor | | |
| brivaracetam | duloxetine | lacosamide | pimozide | tolbutamide |
| cabozantinib | efavirenz | lansoprazole | piroxicam | tolterodine |
| capecitabine | elagolix | lesinurad | ponatinib | tramadol |
| cariprazine | elbasvir/ | letrozole | prasugrel | trimipramine |
| | grazoprevir | | | |
| carisoprodol | eliglustat | lofexidine | propafenone | tropisetron |
| carvedilol | eltrombopag | lomitapide | propranolol | umeclidinium |
| celecoxib | enzalutamide | meclizine | protriptyline | valbenazine |
| ceritinib | erdafitinib | meloxicam | quetiapine | venetoclax |
| cevimeline | escitalopram | methylphenidate | quinidine | venlafaxine |
| citalopram | esomeprazole | metoclopramide | quinine | voriconazole |
| clobazam | everolimus | metoprolol | rabeprazole | vortioxetine |
| clomipramine | fesoterodine | midostaurin | ranolazine | warfarin |
| clonidine | flecainide | mirabegron | regorafenib | zuclopenthixol |
| clopidogrel | flibanserin | mirtazapine | risperidone | |
| clozapine | fluoxetine | moclobemide | rosuvastatin | |

In various embodiments, the plurality of primer pairs is any one selected from Table 3.

TABLE 3
Primers

| SEQ ID NO: | Sequence (5′ to 3′) |
|---|---|
| 1 | GACCTGATGCACCGGCG |
| 2 | ATGTATAAATGCCCTTCTC |
| 3 | TTGCGCAACTTGGGCCTG |
| 4 | ACCCACCGGAGTGGTTG |
| 5 | GCCGCCTTCGCCAACCAC |
| 6 | ACGGCTTTGTCCAAGAGAC |
| 7 | GTCCTCGTCCTCCTGCAT |
| 8 | TCAGTCAGGTCTCGGGGG |
| 9 | CCGTTCTGTCCCGAGTATG |
| 10 | GGTCACCATCCCGGCAGA |
| 11 | CGTGAGCCCATCTGGGAAA |
| 12 | GAGGTCAGGCTTACAGGAT |
| 13 | ACCATGGTGTCTTTGCTTTCC |
| 14 | GTGAGCAGGGGACCCGA |
| 15 | GTGTCCAGAGGAGCCCAT |
| 16 | GTGGCAGGGGGCTTGGT |
| 17 | GTGTTCCTGGCGCGCTAT |
| 18 | GTAAGGGGTCGCCTTCC |
| 19 | AGGCCTTCCTGGCAGAGAT |
| 20 | TCATTCCTCCTGGGACGC |
| 21 | AGGATCCTGTAAGCCTGAC |
| 22 | ATGAATCACGGCAGTGGTGT |
| 23 | AGGGCCACTTTGTGAAGCC |
| 24 | CAGGAAAGCAAAGACACCATG |
| 25 | GCGTTTCTCCCTCATGAC |
| 26 | GGTCAGTGATATGGAGTAGG |
| 27 | CTGCATGCAAGACAGGAG |
| 28 | CCTTGGGAATGAGATAGTTTCTG |
| 29 | CAGATATGCAATAATTTTCCCAC |
| 30 | GCAAGGTTTTTAAGTAATTTGTTATG |
| 31 | CCATTATTTTCCAGAAACGTTTCG |
| 32 | GGATTTCCCAGAAAAAAAGACTG |
| 33 | AACAAAGTTTTAGCAAACGATTT |
| 34 | ATGCCCATCGTGGCGCA |
| 35 | GGCTCTTATCTACATAGGTTGTT |
| 36 | CTATGGGAGTCTCCCCTATT |

In various embodiments, the probe for carrying out the real-time PCR assay is any one selected from Table 4.

TABLE 4
Probes

| SEQ | |
|---|---|
| ID NO: | Sequence (5′ to 3′) |
| 37 | /56-FAM/CTGGTGGGTAGCGTGCA/3BHQ_1/ |
| 38 | /5HEX/CCTGGTGAGTAGCGTGCAG/3IABKFQ/ |
| 39 | /56-FAM/TCGGTCACCCACTGCTCCAG/3IABKFQ/ |
| 40 | /5HEX/TCGGTCACCCCTGCTCCAG/3IABKFQ/ |
| 41 | /56-FAM/ACCCCCAGGACGCCCCTT/3IABKFQ/ |
| 42 | /5HEX/ACCCCCAAGACGCCCCTTT/3IABKFQ/ |
| 43 | /56-FAM/TCCCAGGTCATCCTGTGCTCA/3BHQ_1/ |
| 44 | /5HEX/CAGGTCATCCGTGCTCAG/3IABKFQ/ |
| 45 | /56-FAM/AGCCACCACTATGCGCAGGT/3BHQ_1/ |
| 46 | /5HEX/AGCCACCACTATGCACAGGT/3IABKFQ/ |
| 47 | /56-FAM/AGGGAGGAAGGGTACAGGC/3BHQ_1/ |
| 48 | /5HEX/AGGGAGAAAGGGTACAGGC/3IABKFQ/ |
| 49 | /56-FAM/TGGTGAGCCCATCCCCCTAT/3BHQ_1/ |
| 50 | /5HEX/TGGTGACCCCATCCCCCTAT/3IABKFQ/ |
| 51 | /56-FAM/TGGTGCCCCTGGCCGTGATA/3BHQ 1/ |
| 52 | /5HEX/TGGTGCCCCTGGCCATGATA/3IABKFQ/ |
| 53 | /56-FAM/TCGCCAACCACTCCGGTGG/3IABKFQ/ |
| 54 | /5HEX/TCGCCAACCACTCCAGTGG/3IABKFQ/ |
| 55 | /5Cy5/TCGCCAACCACTCCTGTGG/31AbRQSp/ |
| 56 | /56-FAM/AGAGATGGAGAAGGTGAGAGTG/3IABKFQ/ |
| 57 | /5HEX/AGAGATGGAGGTGAGAGTG/3IABKFQ/ |
| 58 | /56-FAM/ATCGACGACGTGATAGGGCAG/3IABKFQ/ |
| 59 | /5HEX/ATCGACGACATGATAGGGCAG/3IABKFQ/ |
| 60 | /56-FAM/CACAGGCCGCCGTGCATG/3BHQ_1/ |
| 61 | /5HEX/CCACAGGCCACCGTGCATG/3IABKFQ/ |
| 62 | /56-FAM/CATTGAGGACCGTGTTCAAGAG/3BHQ_1/ |
| 63 | /5HEX/CATTGAGGACTGTGTTCAAGAG/3BHQ_1/ |
| 64 | /56-FAM/CGAGGTCCAGAGATACATTGA/3BHQ_1/ |
| 65 | /5HEX/CGAGGTCCAGAGATACCTTGA/3IABKFQ/ |
| 66 | /56-FAM/TCATTGATTATTTCCCGGGAAC/3BHQ_1/ |
| 67 | /5HEX/TCATTGATTATTTCCCAGGAAC/3IABKFQ/ |
| 68 | /56-FAM/TAAGCACCCCCTGGATCCAGG/3IABKFQ/ |
| 69 | /5HEX/TAAGCACCCCCTGAATCCAGG/3IABKFQ/ |
| 70 | /56-FAM/TCTTCTGTTCTCAAAGCATC/3BHQ_1/ |
| 71 | /5HEX/TGTCTTCTGTTCTCAAAGTA/3IABKFQ/ |
| 72 | /56-FAM/TATGTGTTCATGGGTAATATGCT/3BHQ_1/ |
| 73 | /5HEX/ATATGCGTTCATGGGTAATATG/3IABKFQ/ |

In various embodiments, the plurality of primer pairs and probes is any one selected from the list in Tables 3 and 4.

Table 5 below shows the various primers and probes used for carrying out the relevant assays to detect the respective variants.

TABLE 5

| | | Primers and probes used |
|---|---|---|
| Gene | Variant | (SEQ ID NOs:) |
| CYP2D6 | rs1065852 | 1-2, 37-38 |
| | rs5030655 | 3-4, 39-40 |
| | rs3892097 | 5-6, 41-42 |
| | rs35742686 | 7-8, 43-44 |
| | rs16947 | 9-10, 45-46 |
| | rs28371725 | 11-12, 47-48 |
| | rs1135840 | 13-14, 49-50 |
| | rs769258 | 15-16, 51-52 |
| | rs5030865 | 17-18, 53-55 |
| | rs5030656 | 19-20, 56-57 |
| | rs59421388 | 21-22, 58-59 |
| | rs267608319 | 23-24, 60-61 |
| | exon 9 conversion (*36) | Commercially obtained |
| | deletion (*5) | Commercially obtained |
| CYP2C9 | rs1799853 | 25-26, 62-63 |
| | rs1057910 | 27-28, 64-65 |
| CYP2C19 | rs4244285 | 29-30, 66-67 |
| | rs4986893 | 31-32, 68-69 |
| | rs12248560 | 33-34, 70-71 |
| SLCO1B1 | rs4149056 | 35-36, 72-73 |

In various embodiments, the single real-time polymerase chain reaction run of this invention comprises 50 cycles of denaturation and annealing/extension, said denaturation is carried out at about 95° C. for about 15 seconds and said annealing/extension is carried out at about 60° C. for about 60 seconds.

In another aspect of the invention, there is provided a kit comprising means for screening or evaluating a human subject's response to an administration of a plurality of therapeutic agents by determining genotype of the subject in a sample containing subject's nucleic acid. Such means include any one of those primer pairs set out in Table 3.

Advantageously, this invention provides a pharmacogenomics test that combines multiple variants to be tested together under the same real-time PCR conditions that can be prescribed in outpatient settings or through General Practitioners. In addition, this test considers variants prevalent in minority ethnicities to ensure wider use adoption in Asian primary care settings. In order that the present invention may be fully understood and readily put into practical effect, there shall now be described by way of non-limitative examples only preferred embodiments of the present invention, the description being with reference to the accompanying illustrative figures.

In the Figures:

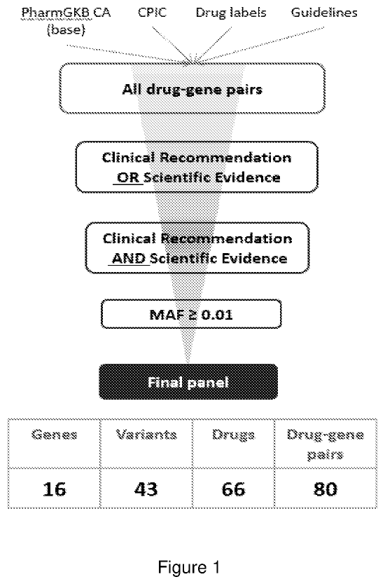

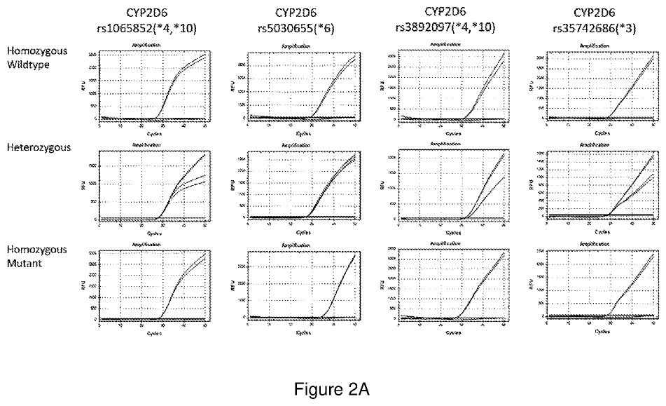

FIG. 1 is a workflow showing the designing of the various pharmacogenomic markers for carrying out the assay of the invention.

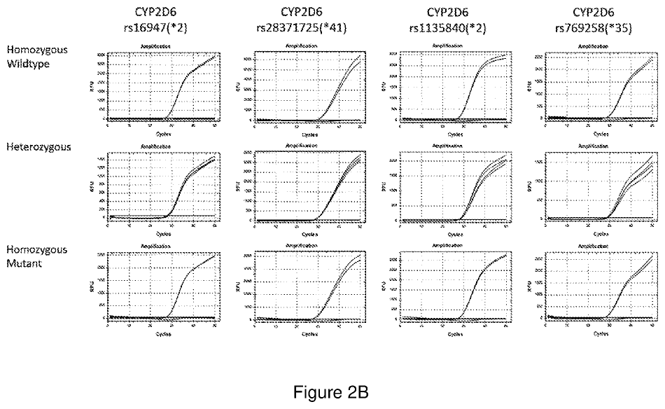

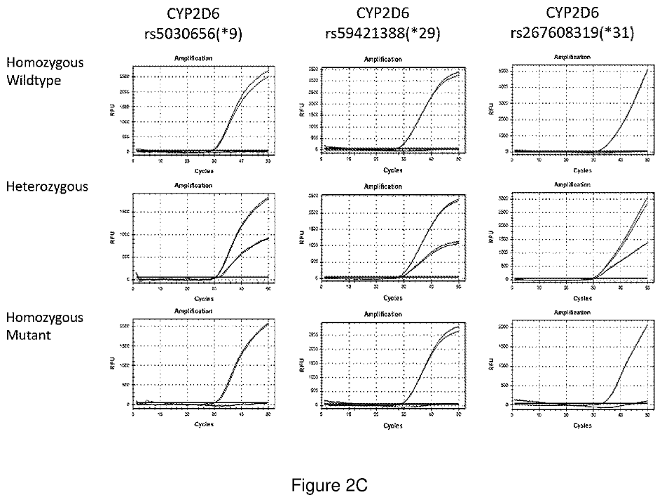

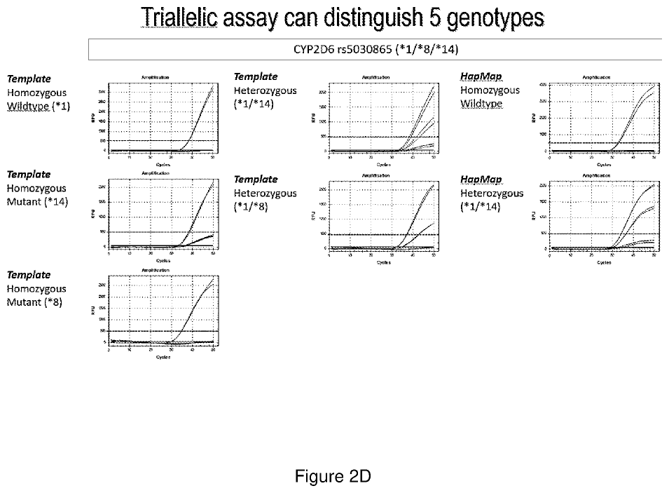

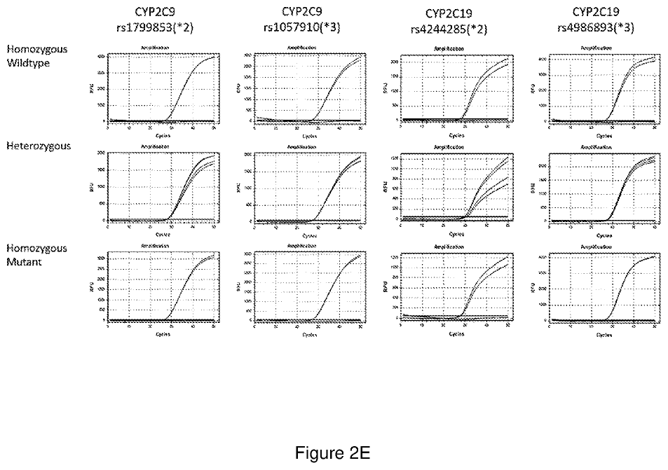

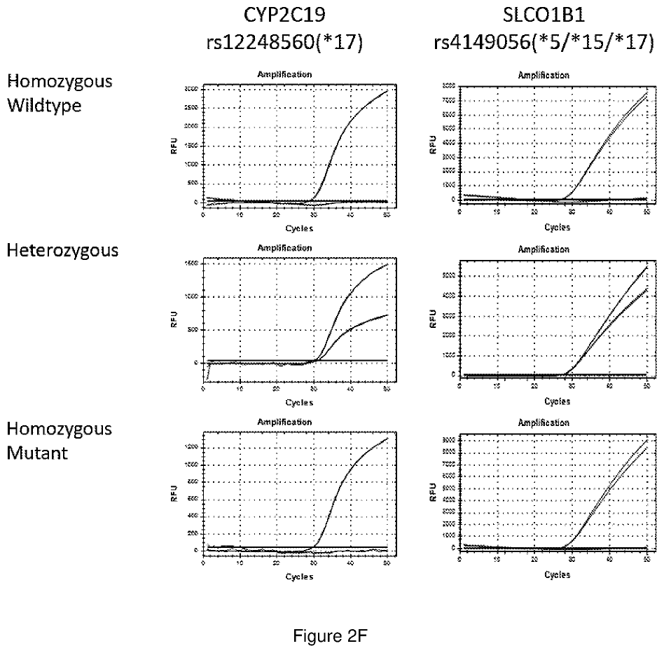

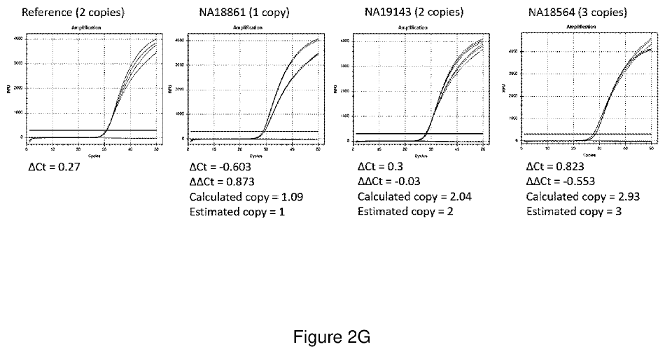

FIGS. 2A to 2G show final output results based on the various assay designs, tested on multiple HapMap samples with known genotypes. Performance of completed assays on multiple genotypes demonstrate that assays are able to accurately discriminate between expected genotypes, i.e. homozygous wildtype samples only show amplification in the FAM channel, heterozygous samples show amplification in both the FAM and HEX channels or FAM and Cy⁵ channels, and homozygous mutant samples only show amplification in the HEX channel or Cy5 channel.

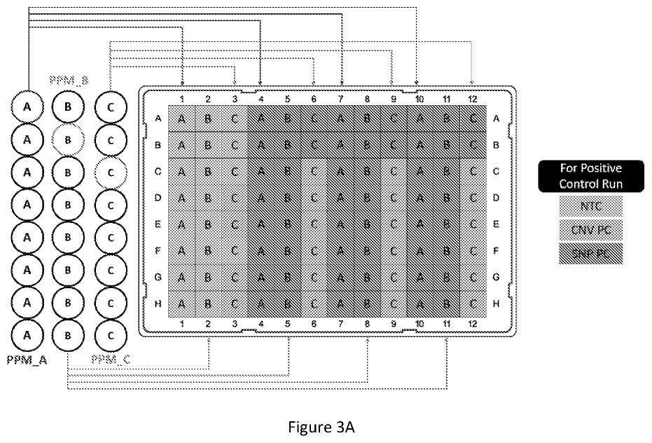

FIGS. 3A and 3B are schematic drawings showing Positive Control (PC) plate layout (FIG. 3A) and Sample plate (FIG. 3B).

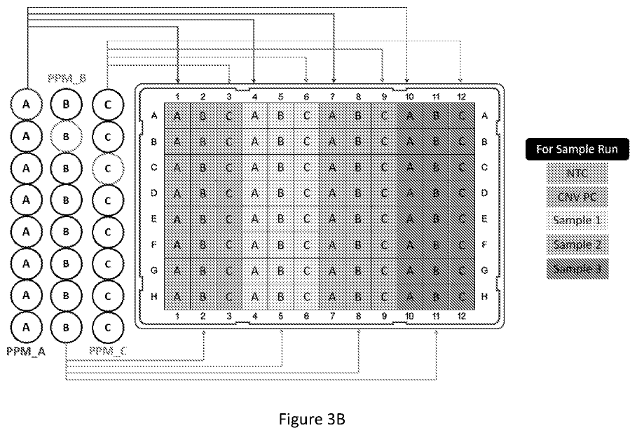

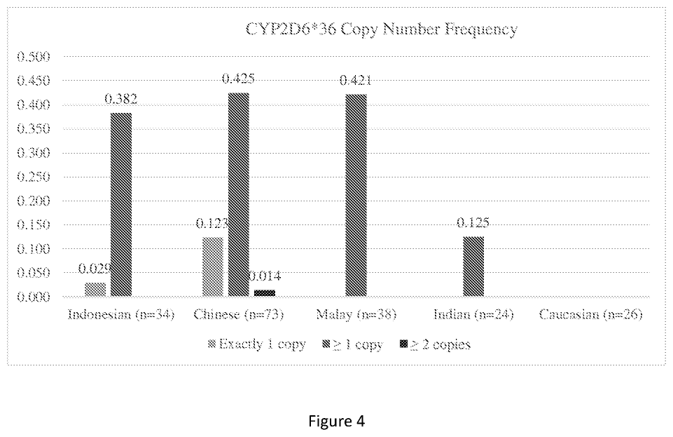

FIG. 4 shows the CYP2D6*36 frequency by ethnicity. The figure shows the distribution of individuals carrying exactly one, one or more, or two or more copies of the CYP2D6*36 allele among the study cohort (n=195), grouped per ethnicity.

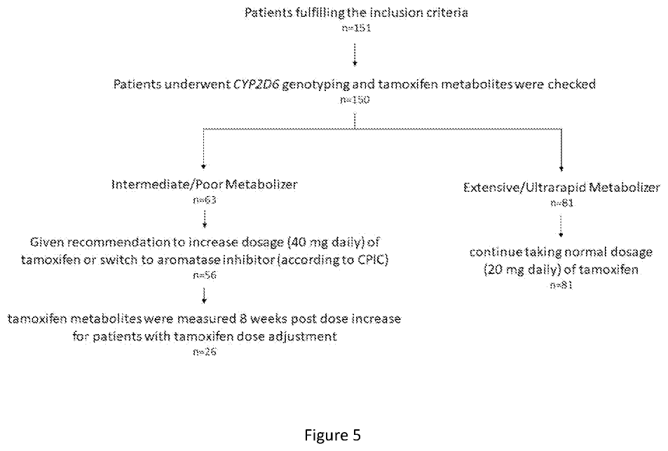

FIG. 5 shows a research flow diagram for the clinical validation of the Nala Core PGx Core™ kit used for CYP2D6 genotyping for personalised therapy of tamoxifen in breast cancer patients.

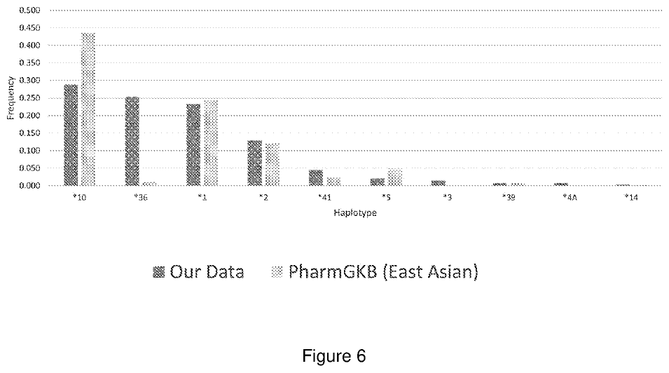

FIG. 6 shows the distribution of haplotype frequencies among Indonesian breast cancer patients (n=288).

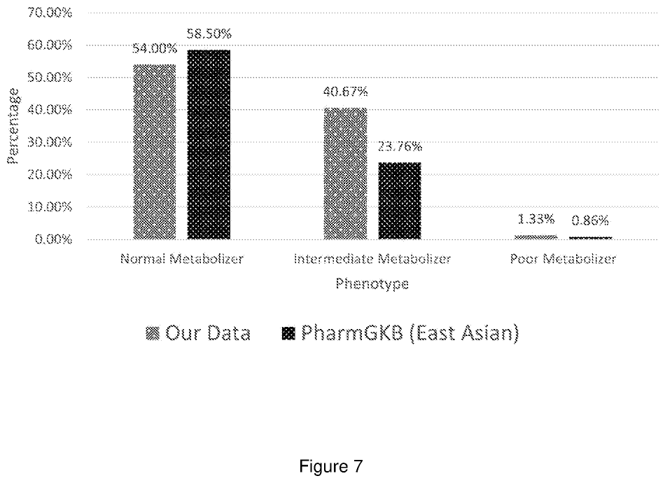

FIG. 7 shows the distribution of phenotype frequencies among Indonesian breast cancer patients (n=144).

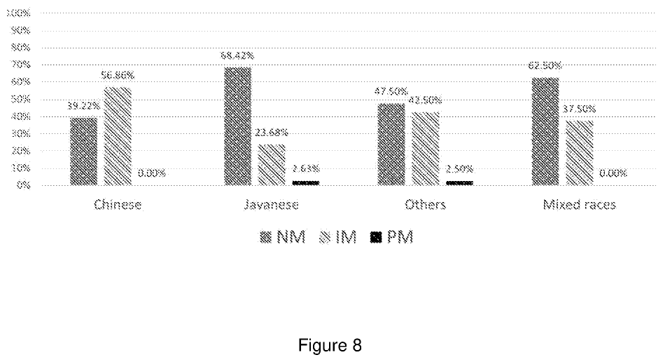

FIG. 8 shows the distribution of phenotype frequencies per major ethnicity among Indonesian breast cancer patients (n=151).

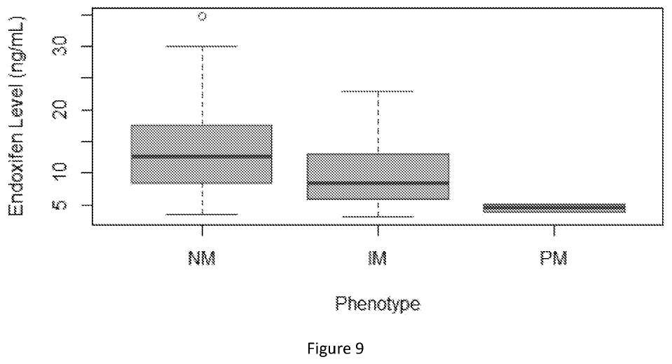

FIG. 9 shows the distribution of endoxifen levels for each observed phenotype at the baseline. Normal metabolizer/NM (n=81), Intermediate metabolizer/IM (n=61), Poor Metabolizer/PM (n=2).

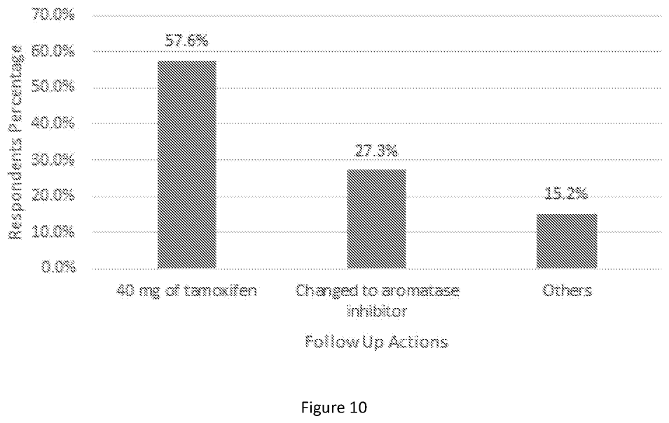

FIG. 10 shows the distribution of the different follow up actions selected by doctors after patient's CYP2D6 profile was characterized through genetic testing (n=66).

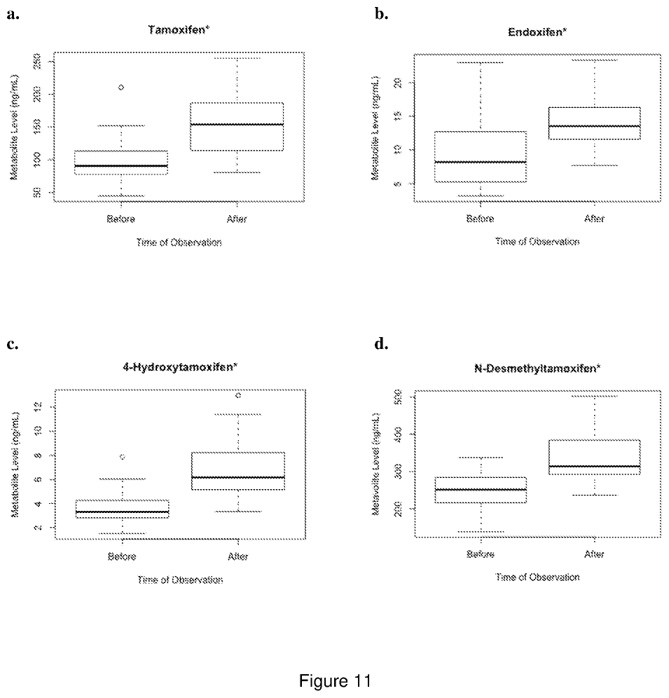

FIG. 11 shows the metabolite levels before and after dose adjustment for IM patients. a) Tamoxifen, b) endoxifen, c) 4-hydroxytamoxifen, d) N-desmethyltamoxifen. *Statistically significant p-values were observed between metabolites before and after dose adjustment (n=26).

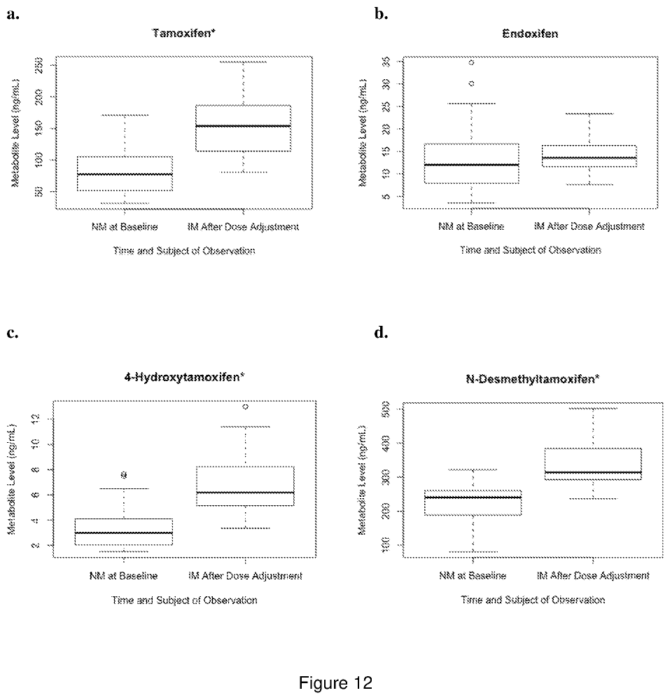

FIG. 12 shows the metabolite levels in IMs after dose adjustment compared to NMs at the baseline. a) Tamoxifen, b) endoxifen, c) 4-hydroxytamoxifen, d) N-desmethyltamoxifen. *Statistically significant p-values were observed, n=81 (NMs), n=26 (IMs). Endoxifen levels in IMs post dose adjustment were statistically similar to NMs at the baseline.

In devising this invention, various pharmacogenomic markers that may be relevant to screening in Asians were identified and curated. The reagent cocktail for all variants were then designed, developed and tested. This was then followed by optimizing the reagents and conditions for all variants used in the assays. Each process is briefly described below.

### 1. Curating Pharmacogenomic Markers Relevant to Screening in Asians

Briefly, the curation and prioritization process was as follows:
a) Shortlisting of variants related to drug-gene pairs that already had at least one clinical recommendation, which was defined as:
   i. having existing guidelines from at least one of the following: Clinical Pharmacogenomics Implementation Consortium (CPIC), Dutch Pharmacogenomic Working Group (DPWG), Canadian Pharmacogenomics Network for Drug Safety (CPNDS), or professional society (PRO)
   ii. having actionable labels from U.S. Food and Drug Administration (FDA), European Medicines Agency (EMA), Health Canada (Sante Canada) (HCSC), Pharmaceuticals and Medical Devices Agency, Japan (PMDA)
   iii. having CPIC annotations levels of either A or B, i.e. drug-gene pairs had high clinical context which means that genetic information was highly recommended to be used to change prescribing of affected drugs.
b) Shortlisting of variants that were annotated with strong scientific evidence. PharmGKB database provides clinical annotations of each drug response variant according to its published scientific evidence (effect size and P-value) and availability of medical society-endorsed PGx guidelines. Only those with PharmGKB levels 1A, 1B and 2A were taken as a cut-off indication for strong scientific evidence.
c) Shortlisting of variants that were present in at least one of these populations (Chinese, Malays, Indians, Caucasians), with a minor allele frequency of 1% or greater. The sources for population frequency data included the Singapore Genome Variation Project (SGVP), 1000 Genomes Project, Exome Aggregation Consortium (ExAC) project and GIS' internal data.

These curation steps resulted in a panel that consisted of 16 genes, 43 variants, 66 drugs, 80 drug-gene pairs. This workflow is summarized in FIG. 1.

Further work was done for the curation of variants to be applicable for outpatient settings (general practitioners) by obtaining data related to drugs and adverse events collected in Singapore and Asia. Drugs with high likelihood of genetic association and burden to the society were included in the panel. The biomarker to predict the risk of adverse events and low efficacy from those drugs were obtained considering strength of scientific evidence and predictive power. This set of drug-gene and variants were designed as the main panel which was designated “NalaPGx Core™”.

The drug and gene list for NalaPGx Core™ are shown in Table 6 below.

TABLE 6

| Drug name | Gene | Drug Classification | Indication |
|---|---|---|---|
| Losartan | CYP2C9 | Agents Acting On The | Management of hypertension |
| | | Renin-Angiotensin System | |
| Codeine and | CYP2D6 | Analgesics | Management of pain |
| Paracetamol | | | |
| Eletriptan | CYP2D6 | Analgesics | Management of pain |
| Oxycodone | CYP2D6 | Analgesics | Management of pain |
| Paracetamol, | CYP2D6 | Analgesics | Management of pain |
| Combinations Excl. | | | |
| Psycholeptics | | | |
| Tramadol | CYP2D6 | Analgesics | Management of pain |
| Oliceridine | CYP2D6 | Analgesics | Management of pain |
| Rimegepant | CYP2C9 | Analgesics | Management of pain |
| Paracetamol, | CYP2D6 | Analgesics | Management of pain |
| Caffeine and | | | |
| Dihydrocodeine | | | |
| Dronabinol | CYP2C9 | Antiemetics And | Management of anorexia |
| | | Antinauseants | |
| Ondansetron | CYP2D6 | Antiemetics And | Prevent nausea and vomiting |
| | | Antinauseants | |
| Palonosetron | CYP2D6 | Antiemetics And | Prevent nausea and vomiting |
| | | Antinauseants | |
| Tropisetron | CYP2D6 | Antiemetics And | Prevent nausea and vomiting |
| | | Antinauseants | |
| Brivaracetam | CYP2C19 | Antiepileptics | Management of seizures |
| Brivaracetam | CYP2C9 | Antiepileptics | Management of seizures |
| Lacosamide | CYP2C19 | Antiepileptics | Management of seizures |
| Phenytoin | CYP2C9 | Antiepileptics | Management of seizures |
| Phenytoin | CYP2C19 | Antiepileptics | Management of seizures |
| Terbinafine | CYP2D6 | Antifungals For | Management of fungal skin and |
| | | Dermatological Use | nail infections |
| Lesinurad | CYP2C9 | Antigout Preparations | Management of hyperuricemia |
| Avatrombopag | CYP2C9 | Antihemorrhagics | Management of thrombocytopenia |
| Meclozine | CYP2D6 | Antihistamines For | Management of nausea, vomiting, |
| | | Systemic Use | dizziness and vertigo |
| Clonidine | CYP2D6 | Antihypertensives | Treatment of hypertension |
| Celecoxib | CYP2C9 | Antiinflammatory And | Symptomatic treatment of |
| | | Antirheumatic Products | inflammatory, musculoskeletal |
| | | | and rheumatic disorders |
| Flurbiprofen | CYP2C9 | Antiinflammatory And | Symptomatic treatment of |
| | | Antirheumatic Products | inflammatory, musculoskeletal |
| | | | and rheumatic disorders |
| Ibuprofen | CYP2C9 | Antiinflammatory And | Symptomatic treatment of |
| | | Antirheumatic Products | inflammatory, musculoskeletal |
| | | | and rheumatic disorders |
| Lornoxicam | CYP2C9 | Antiinflammatory And | Symptomatic treatment of |
| | | Antirheumatic Products | inflammatory, musculoskeletal |
| | | | and rheumatic disorders |
| Meloxicam | CYP2C9 | Antiinflammatory And | Symptomatic treatment of |
| | | Antirheumatic Products | inflammatory, musculoskeletal |
| | | | and rheumatic disorders |
| Piroxicam | CYP2C9 | Antiinflammatory And | Symptomatic treatment of |
| | | Antirheumatic Products | inflammatory, musculoskeletal |
| | | | and rheumatic disorders |
| Tenoxicam | CYP2C9 | Antiinflammatory And | Symptomatic treatment of |
| | | Antirheumatic Products | inflammatory, musculoskeletal |
| | | | and rheumatic disorders |
| Voriconazole | CYP2C19 | Antimycotics For | Management of fungal infections |
| | | Systemic Use | |
| Axitinib | CYP2C19 | Antineoplastic Agents | Prevent the proliferation of neoplasms |
| Erdafitinib | CYP2C9 | Antineoplastic Agents | Prevent the proliferation of neoplasms |
| Gefitinib | CYP2D6 | Antineoplastic Agents | Prevent the proliferation of neoplasms |
| Ibrutinib | CYP2D6 | Antineoplastic Agents | Prevent the proliferation of neoplasms |
| Rucaparib | CYP2D6 | Antineoplastic Agents | Prevent the proliferation of neoplasms |
| Quinine | CYP2D6 | Antiprotozoals | Treatment of malaria and leg cramps |
| Acenocoumarol | CYP2C9 | Antithrombotic Agents | Treatment and prevention of |
| | | | thromboembolic diseases |
| Clopidogrel | CYP2C19 | Antithrombotic Agents | Prevention of blood clots in peripheral |
| | | | vascular disease, coronary artery |
| | | | disease, and cerebrovascular disease |
| Phenprocoumon | CYP2C9 | Antithrombotic Agents | Prevention and treatment of |
| | | | thromboembolic disease |
| Prasugrel | CYP2C9, | Antithrombotic Agents | Reduce risk of thrombotic |
| | CYP2C19 | | cardiovascular events |
| Ticagrelor | CYP2C19 | Antithrombotic Agents | Reduce the risk of cardiovascular |
| | | | death, myocardial infarction, and stroke |
| Warfarin | CYP2C9 | Antithrombotic Agents | Treatment of venous thromboembolism, |
| | | | pulmonary embolism, thromboembolism |
| | | | with atrial fibrillation, thromboembolism |
| | | | with cardiac valve replacement, and |
| | | | thromboembolic events post |
| | | | myocardial infarction |
| Atazanavir | CYP2C19 | Antivirals For Systemic Use | Treatment of HIV-1 infections |
| Letermovir | SLCO1B1 | Antivirals For Systemic Use | Treatment of cytomegalovirus (CMV) |
| | | | infections |
| Nelfinavir | CYP2C19 | Antivirals For Systemic Use | Treatment of HIV infections |
| Ritonavir | CYP2D6 | Antivirals For Systemic Use | Treatment of HIV-1 infections |
| Atenolol | CYP2D6 | Beta Blocking Agents | Management of hypertension and |
| | | | chronic angina |
| Bisoprolol | CYP2D6 | Beta Blocking Agents | Treatment of hypertension |
| Carvedilol | CYP2D6 | Beta Blocking Agents | Treatment of chronic heart failure, |
| | | | hypertension, and left ventricular |
| | | | dysfunction |
| Metoprolol | CYP2D6 | Beta Blocking Agents | Treatment of angina, heart failure, |
| | | | myocardial infarction, atrial fibrillation, |
| | | | atrial flutter and hypertension |
| Nebivolol | CYP2D6 | Beta Blocking Agents | Treatment of hypertension |
| Propranolol | CYP2D6 | Beta Blocking Agents | Treatment of hypertension |
| Sotalol | CYP2D6 | Beta Blocking Agents | Treatment of life threatening |
| | | | ventricular arrhytmias and maintain |
| | | | normal sinus rhythm in patients with |
| | | | atrial fibrillation or flutter |
| Timolol | CYP2D6 | Beta Blocking Agents | Treatment of increased intraocular |
| | | | pressure associated with ocular |
| | | | hypertension or open-angle glaucoma |
| Amiodarone | CYP2D6 | Cardiac Therapy | Treatment of recurrent ventricular |
| | | | fibrillation (VF) and recurrent |
| | | | hemodynamically unstable ventricular |
| | | | tachycardia (VT). |
| Disopyramide | CYP2D6 | Cardiac Therapy | Treatment of ventricular arrhythmias |
| Dronedarone | CYP2D6 | Cardiac Therapy | Management of paroxysmal or |
| | | | persistent atrial fibrillation |
| Flecainide | CYP2D6 | Cardiac Therapy | Management of atrial fibrillation and |
| | | | paroxysmal supraventricular |
| | | | tachycardias (PSVT). |
| Propafenone | CYP2D6 | Cardiac Therapy | Management of paroxysmal atrial |
| | | | fibrillation/flutter and ventricular |
| | | | arrhythmias |
| Quinidine | CYP2D6 | Cardiac Therapy | Treatment of ventricular pre-excitation |
| | | | and cardiac dysrhythmias |
| Ranolazine | CYP2D6 | Cardiac Therapy | Treatment of chronic angina |
| Vernakalant | CYP2D6 | Cardiac Therapy | Treatment of atrial fibrillation |
| Codeine | CYP2D6 | Cough And Cold Preparations | Management of pain |
| Dextromethorphan | CYP2D6 | Cough And Cold Preparations | Treatment of coughs and upper |
| | | | respiratory symptoms |
| Opium Derivatives | CYP2D6 | Cough And Cold Preparations | Management of pain |
| and Expectorants | | | |
| Hydrocodone | CYP2D6 | Cough And Cold Preparations | Management of pain |
| Dexlansoprazole | CYP2C19 | Drugs For Acid Related | Treatment of erosive esophagitis and |
| | | Disorders | relief of heartburn |
| Esomeprazole | CYP2C19 | Drugs For Acid Related | Treatment of acid-reflux disorders |
| | | Disorders | |
| Lansoprazole | CYP2C19 | Drugs For Acid Related | Reduction of gastric acid secretion |
| | | Disorders | |
| Omeprazole | CYP2C19 | Drugs For Acid Related | Treatment of acid-reflux disorders |
| | | Disorders | |
| Pantoprazole | CYP2C19 | Drugs For Acid Related | Treatment of acid-reflux disorders |
| | | Disorders | |
| Rabeprazole | CYP2C19 | Drugs For Acid Related | Treatment of acid-reflux disorders |
| | | Disorders | |
| Metoclopramide | CYP2D6 | Drugs For Functional | Treatment of recurrent diabetic |
| | | Gastrointestinal Disorders | gastroparesis |
| Arformoterol | CYP2D6 | Drugs For Obstructive | Treatment of airflow obstruction |
| | | Airway Diseases | |
| Formoterol | CYP2C19 | Drugs For Obstructive | Treatment of airflow obstruction |
| | | Airway Diseases | |
| Formoterol | CYP2D6 | Drugs For Obstructive | Treatment of airflow obstruction |
| | | Airway Diseases | |
| Tiotropium | CYP2D6 | Drugs For Obstructive | Treatment of airflow obstruction |
| Bromide | | Airway Diseases | |
| Vilanterol and | CYP2D6 | Drugs For Obstructive | Treatment of airflow obstruction |
| Umeclidinium | | Airway Diseases | |
| Bromide | | | |
| Umeclidinium | CYP2D6 | Drugs For Obstructive | Treatment of airflow obstruction |
| Bromide | | Airway Diseases | |
| Glibenclamide | CYP2C9 | Drugs Used In Diabetes | Management of hyperglycemia |
| Gliclazide | CYP2C9 | Drugs Used In Diabetes | Management of hyperglycemia |
| Glimepiride | CYP2C9 | Drugs Used In Diabetes | Management of hyperglycemia |
| Tolbutamide | CYP2C9 | Drugs Used In Diabetes | Management of hyperglycemia |
| Tamoxifen | CYP2D6 | Endocrine Therapy | Management of estrogen receptor |
| | | | positive metastatic breast cancer |
| Siponimod | CYP2C9 | Immunosuppressants | Management of relapsing multiple sclerosis |
| Upadacitinib | CYP2D6 | Immunosuppressants | Treatment of active rheumatoid |
| | | | arthritis or active psoriatic arthritis |
| Amlodipine, | SLCO1B1 | Lipid Modifying Agents | Management of hypertension and angina |
| Atorvastatin, | | | |
| and Perindopril | | | |
| Arginine | | | |
| Atorvastatin | SLCO1B1 | Lipid Modifying Agents | Treatment of hyperlipidemia |
| Rosuvastatin | SLCO1B1 | Lipid Modifying Agents | Treatment of hyperlipidemia |
| and Ezetimibe | | | |
| Simvastatin and | SLCO1B1 | Lipid Modifying Agents | Treatment of hyperlipidemia |
| Ezetimibe | | | |
| Fenofibrate | SLCO1B1 | Lipid Modifying Agents | Treatment of hyperlipidemia |
| Fluvastatin | SLCO1B1 | Lipid Modifying Agents | Treatment of hyperlipidemia |
| Pitavastatin | SLCO1B1 | Lipid Modifying Agents | Treatment of hyperlipidemia |
| Rosuvastatin | SLCO1B1 | Lipid Modifying Agents | Treatment of hyperlipidemia |
| Simvastatin | SLCO1B1 | Lipid Modifying Agents | Treatment of hyperlipidemia |
| Carisoprodol | CYP2C19 | Muscle Relaxants | Relief of discomfort associated with |
| | | | various musculoskeletal conditions |
| Eliglustat | CYP2D6 | Other Alimentary Tract | Treatment of type 1 Gaucher disease |
| | | And Metabolism Products | |
| Flibanserin | CYP2C19 | Other Gynecologicals | Treatment of hypoactive sexual desire |
| | | | disorder (HSDD) in premenopausal women |
| Flibanserin | CYP2C9 | Other Gynecologicals | Treatment of hypoactive sexual desire |
| | | | disorder (HSDD) in premenopausal women |
| Flibanserin | CYP2D6 | Other Gynecologicals | Treatment of hypoactive sexual desire |
| | | | disorder (HSDD) in premenopausal women |
| Cevimeline | CYP2D6 | Other Nervous System Drugs | Treatment of symptoms of dry mouth |
| | | | associated with Sjögren's Syndrome. |
| Deutetrabenazine | CYP2D6 | Other Nervous System Drugs | Treatment of tardive dyskinesia and chorea |
| | | | associated with Huntington's disease. |
| Dextromethorphan | CYP2D6 | Other Nervous System Drugs | Treatment of pseudobulbar affect |
| and Quinidine | | | |
| Lofexidine | CYP2D6 | Other Nervous System Drugs | Management of symptoms associated |
| | | | with acute withdrawal from opioids |
| Pitolisant | CYP2D6 | Other Nervous System Drugs | Management of narcolepsy |
| Tetrabenazine | CYP2D6 | Other Nervous System Drugs | Management of chorea associated |
| | | | with Huntington's Disease. |
| Valbenazine | CYP2D6 | Other Nervous System Drugs | Treatment of tardive dyskinesia |
| Methadone | CYP2D6 | Other Nervous System Drugs | Detoxification treatment of opioid |
| | | | addiction |
| Elagolix | SLCO1B1 | Pituitary And Hypothalamic | Treatment of pain in endometriosis. |
| | | Hormones And Analogues | |
| Amitriptyline | CYP2C19, | Psychoanaleptics | Management of depressive illness |
| | CYP2D6 | | |
| Amoxapine | CYP2D6 | Psychoanaleptics | Management of depressive disorders |
| | | | and psychotic depression |
| Amfetamine | CYP2D6 | Psychoanaleptics | Treatment of Attention Deficit |
| | | | Hyperactivity Disorder (ADHD) |
| Atomoxetine | CYP2D6 | Psychoanaleptics | Management of Attention Deficit |
| | | | Hyperactivity Disorder (ADHD) |
| Citalopram | CYP2D6, | Psychoanaleptics | Treatment of depression |
| | CYP2C19 | | |
| Clomipramine | CYP2C19, | Psychoanaleptics | Treatment of obsessive-compulsive disorders |
| | CYP2D6 | | |
| Desipramine | CYP2D6 | Psychoanaleptics | Treatment of depression |
| Desvenlafaxine | CYP2D6 | Psychoanaleptics | Treatment of major depressive disorders |
| Donepezil | CYP2D6 | Psychoanaleptics | Treatment of behavioral and cognitive |
| | | | effects of Alzheimer's Disease and |
| | | | other types of dementia |
| Doxepin | CYP2C19, | Psychoanaleptics | Treatment of depression, anxiety, |
| | CYP2D6 | | manic-depressive disorder, and insomnia |
| Duloxetine | CYP2D6 | Psychoanaleptics | Treatment of anxiety disorder, |
| | | | neuropathic pain, osteoarthritis, |
| | | | and stress incontinence |
| Escitalopram | CYP2C19 | Psychoanaleptics | Treatment of major depressive |
| | | | disorder, generalized anxiety disorder, |
| | | | and other select psychiatric disorders |
| Fluoxetine | CYP2D6 | Psychoanaleptics | Management of major depressive |
| | | | disorder, obsessive compulsive |
| | | | disorder, and bulimia nervosa |
| Fluoxetine and | CYP2D6 | Psychoanaleptics | Treatment of depression related to |
| Olanzapine | | | Bipolar I Disorder, and treatment |
| | | | resistant depression |
| Fluvoxamine | CYP2D6 | Psychoanaleptics | Management of depression and for |
| | | | Obsessive Compulsive Disorder (OCD) |
| Galantamine | CYP2D6 | Psychoanaleptics | Treatment of dementia of the |
| | | | Alzheimer's type |
| Imipramine | CYP2C19, | Psychoanaleptics | Relief of symptoms of depression |
| | CYP2D6 | | |
| Methylphenidate | CYP2D6 | Psychoanaleptics | Management of Attention Deficit |
| | | | Hyperactivity Disorder (ADHD) |
| Mirtazapine | CYP2C19 | Psychoanaleptics | Treatment of major depressive disorder |
| Mirtazapine | CYP2D6 | Psychoanaleptics | Treatment of major depressive disorder |
| Moclobemide | CYP2C19 | Psychoanaleptics | Treatment of major depressive |
| | | | disorder and bipolar disorder |
| Modafinil | CYP2D6 | Psychoanaleptics | Improve wakefulness in patients with |
| | | | excessive daytime sleepiness (EDS) |
| | | | associated with narcolepsy |
| Nefazodone | CYP2D6 | Psychoanaleptics | Treatment of depression |
| Nortriptyline | CYP2D6 | Psychoanaleptics | Treatment of depression |
| Paroxetine | CYP2D6 | Psychoanaleptics | Management of depression, |
| | | | obsessive-compulsive disorder, panic |
| | | | disorder, social anxiety disorder, |
| | | | generalized anxiety disorder, |
| | | | posttraumatic stress disorder |
| Protriptyline | CYP2D6 | Psychoanaleptics | Treatment of depression |
| Sertraline | CYP2C19 | Psychoanaleptics | Management of major depressive disorder, |
| | | | post-traumatic stress disorder, obsessive- |
| | | | compulsive disorder, panic disorder, |
| | | | premenstrual dysphoric disorder, and |
| | | | social anxiety disorder |
| Trimipramine | CYP2C19, | Psychoanaleptics | Treatment of depression |
| | CYP2D6 | | |
| Venlafaxine | CYP2D6 | Psychoanaleptics | Management of major depressive disorder, |
| | | | generalized anxiety disorder, social |
| | | | anxiety disorder, and panic disorder |
| Vortioxetine | CYP2D6 | Psychoanaleptics | Treatment of major depressive disorder |
| Bupropion | CYP2D6 | Psychoanaleptics | Treatment of major depressive disorder, |
| | | | seasonal affective disorder, and as an |
| | | | aid to smoking cessation |
| Aripiprazole | CYP2D6 | Psycholeptics | Management of mood and psychotic disorders |
| Aripiprazole | CYP2D6 | Psycholeptics | Management of schizophrenia |
| lauroxil | | | |
| Brexpiprazole | CYP2D6 | Psycholeptics | Management of schizophrenia and |
| | | | major depressive disorder |
| Cariprazine | CYP2D6 | Psycholeptics | Treatment of schizophrenia and episodes |
| | | | associated with bipolar I disorder |
| Clobazam | CYP2C19 | Psycholeptics | Treatment of epilepsy and seizures |
| | | | associated with Lennox-Gastaut syndrome |
| Clozapine | CYP2D6 | Psycholeptics | Treatment of resistant schizophrenia |
| Diazepam | CYP2C19 | Psycholeptics | Treatment of panic disorders, severe |
| | | | anxiety, alcohol withdrawal, and seizures |
| Flupentixol | CYP2D6 | Psycholeptics | Management of panic disorders, severe |
| | | | anxiety, alcohol withdrawal, and seizures |
| Haloperidol | CYP2D6 | Psycholeptics | Treatment of schizophrenia and |
| | | | other psychoses |
| Iloperidone | CYP2D6 | Psycholeptics | Treatment of schizophrenia |
| Olanzapine | CYP2D6 | Psycholeptics | Management of schizophrenia, bipolar |
| | | | 1 disorder, and agitation |
| Paliperidone | CYP2D6 | Psycholeptics | Treatment of schizophrenia and other |
| | | | schizoaffective or delusional disorders |
| Perphenazine | CYP2D6 | Psycholeptics | Management of the manifestations of |
| | | | psychotic disorders |
| Pimozide | CYP2D6 | Psycholeptics | Management of debilitating of motor |
| | | | and phonic tics associated with |
| | | | Tourette's Disorder |
| Quetiapine | CYP2D6 | Psycholeptics | Management of bipolar disorder, |
| | | | schizophrenia, and major depressive |
| | | | disorder. |
| Risperidone | CYP2D6 | Psycholeptics | Treatment of schizophrenia and |
| | | | irritability associated with autistic |
| | | | disorder |
| Sertindole | CYP2D6 | Psycholeptics | Treatment of schizophrenia |
| Thioridazine | CYP2D6 | Psycholeptics | Treatment of schizophrenia and |
| | | | generalized anxiety disorder |
| Zuclopenthixol | CYP2D6 | Psycholeptics | Management of acute psychoses |
| | | | such as mania or schizophrenia |
| Drospirenone and | CYP2C19 | Sex Hormones And Modulators | Prevention of pregnancy |
| Ethinylestradiol | | Of The Genital System | |
| Ospemifene | CYP2C9 | Sex Hormones And Modulators | Management of dyspareunia and |
| | | Of The Genital System | vaginal dryness |
| Tolperisone | CYP2D6 | Topical Products For | Relieve muscle spasticity |
| | | Joint And Muscular Pain | |
| Dapoxetine | CYP2D6 | Urologicals | Treatment of premature ejaculation |
| Darifenacin | CYP2D6 | Urologicals | Management of overactive bladder |
| Fesoterodine | CYP2D6 | Urologicals | Management of overactive bladder |
| Mirabegron | CYP2D6 | Urologicals | Management of overactive bladder |
| Tamsulosin | CYP2D6 | Urologicals | Symptomatic treatment of benign |
| | | | prostatic hyperplasia |
| Tolterodine | CYP2D6 | Urologicals | Management of overactive bladder |

The following provides a description of the assay development that is suitable for running all gene targets in a single real-time PCR run.

### Assay Development

Basic principle: Real-time PCR-based genetic test to determine the genotype and presence of specific genetic markers in a person's genome, including copy number variations (CNVs), insertion deletions (indels) and single nucleotide polymorphisms (SNPs).

### Overall Description of the Technology:

Primers and probes were designed to amplify specific regions in the human genome that have been known and proven to be important for predicting drug response.

### Features of the SNP and Indel Assays Include:

1. Unique design of forward and reverse primers that amplify each target of interest.
2. Unique design of probe sequences that bind the target of interest. Special modifications were made to the nucleotide sequence of the probes when necessary, which improved probe specificity.
3. All our wild-type-targeting probes were tagged by the FAM fluorophore on their 5′ end, while the mutant-targeting probes were tagged by HEX or Cy5 fluorophore on their 5′ end.
4. Multiple quenchers were also used on different probes at their 3′ end, including BHQ1 or IBFQ.
5. Specific concentration ratio of forward/reverse primers as well as FAM/HEX/Cy5 probes were unique to each assay. At times they may be symmetric, whereby the ratio between forward and reverse primers or between FAM, HEX, Cy5 probes are identical. At other times, asymmetric ratio between the two primers and the two probes were chosen. The difference in these concentrations was meant to provide the most optimum discrimination between wild-type and mutant alleles for clarity in genotyping samples.
6. Unique synthetic double-stranded oligos (‘gBlocks’) were designed to depict a homozygous wild-type and a homozygous mutant signal. These oligos were mixed together to create a heterozygous genotype signal.

### Features of the CNV Assays Include:

1. Unique design of forward and reverse primers that amplify a conserved area of the target gene and a housekeeping gene
2. Unique design of probe sequences that bind the target of interest. Special modifications were made to the nucleotide sequence of the probes when necessary, which improved probe specificity.
3. FAM fluorophore was placed at the 5′ end of the probes that target the gene of interest while VIC fluorophore was placed at the 5′ end of the probes that target the housekeeping gene.
4. FAM probes had 3′ modification of a BHQ1 quencher, while VIC probe was modified with a non-fluorescent quencher on its 3′ end.
5. A commercially available purified genomic DNA product was used to depict a fixed copy number (CN=2) that is used as a ‘reference’.
6. The calculation of total copy number is based on the difference between the Ct values of the FAM and VIC signal (‘ΔCt #1’) and the difference between the Ct values of the unknown sample and the ‘reference’ (‘ΔCt #2’). The difference between ‘ΔCt #1’ and ‘ΔCt #2’ is called ‘ΔΔCt’. The total copy number of our gene of interest is then calculated using this formula 2×2^{-(ΔΔCt+/-SD)}.

Points 1-4 above can also be an adaptation of the use of modified TaqMan CN Assays. The modifications include changing the cycling conditions, reaction volumes, number of replicates, lower input DNA, and qPCR mastermix so that the assay can be run with a streamlined workflow and the same cycling conditions as the rest of the assays for ease of operator use.

In some embodiments, CNV assays may be used for the detection of indels. For example, as a deletion is equivalent to a CNV with a copy number of 0, a CNV assay may be used for the detection of a deletion.

### Kit Development

### Overall Description:

Panel based on the developed assays (above) that is configured to run on a 96-well plate format that can accommodate 3 unknown samples and 1 no template control (NTC). This panel consists of 20 variants in 4 genes (CYP2D6, CYP2C9, CYP2C19, and SLCO1B1) that are related to prescribing information of 32 drugs. The panel is prepared as a kit where primers and probes are pre-mixed in a bulk strip-tube and user must add master mix before distributing it to a set configuration on a 96-well plate (see Table 7 below). Subsequently, user will need to add DNA templates before running it on the real-time PCR machine.

TABLE 7

| | 1 | 2 | 3 | 4 | 5 | 6 | 7 | 8 | 9 | 10 | 11 | 12 |
|---|---|---|---|---|---|---|---|---|---|---|---|---|
| A | CYP2C9*2 | SNP3 | SNP12 | CYP2C9*2 | SNP3 | SNP12 | CYP2C9*2 | SNP3 | SNP12 | CYP2C9*2 | SNP3 | SNP12 |
| | (NTC) | (NTC) | (NTC) | (S1) | (S1) | (S1) | (S2) | (S2) | (S2) | (S3) | (S3) | (S3) |
| B | CYP2C9*3 | SNP4 | SNP13 | CYP2C9*3 | SNP4 | SNP13 | CYP2C9*3 | SNP4 | SNP13 | CYP2C9*3 | SNP4 | SNP13 |
| | (NTC) | (NTC) | (NTC) | (S1) | (S1) | (S1) | (S2) | (S2) | (S2) | (S3) | (S3) | (S3) |
| C | CYP2C19*2 | SNP5 | Int 2 | CYP2C19*2 | SNP5 | Int 2 | CYP2C19*2 | SNP5 | Int 2 | CYP2C19*2 | SNP5 | Int 2 |
| | (NTC) | (NTC) | (NTC) | (S1) | (S1) | (S1) | (S2) | (S2) | (S2) | (S3) | (S3) | (S3) |
| D | CYP2C19*3 | SNP6 | Int 2 | CYP2C19*3 | SNP6 | Int 2 | CYP2C19*3 | SNP6 | Int 2 | CYP2C19*3 | SNP6 | Int 2 |
| | (NTC) | (NTC) | (NTC) | (S1) | (S1) | (S1) | (S2) | (S2) | (S2) | (S3) | (S3) | (S3) |
| E | CYP2C19*17 | SNP7 | Int 2 | CYP2C19*17 | SNP7 | Int 2 | CYP2C19*17 | SNP7 | Int 2 | CYP2C19*17 | SNP7 | Int 2 |
| | (NTC) | (NTC) | (NTC) | (S1) | (S1) | (S1) | (S2) | (S2) | (S2) | (S3) | (S3) | (S3) |
| F | SLCO1B1 | SNP8 | Exon9 | SLCO1B1 | SNP8 | Exon9 | SLCO1B1 | SNP8 | Exon9 | SLCO1B1 | SNP8 | Exon9 |
| | (NTC) | (NTC) | (NTC) | (S1) | (S1) | (S1) | (S2) | (S2) | (S2) | (S3) | (S3) | (S3) |
| G | SNP1 (NTC) | SNP9 | Exon9 | SNP1 (S1) | SNP9 | Exon9 | SNP1 (S2) | SNP9 | Exon9 | SNP1 (S3) | SNP9 | Exon9 |
| | | (NTC) | (NTC) | | (S1) | (S1) | | (S2) | (S2) | | (S3) | (S3) |
| H | SNP2 (NTC) | SNP11 | Exon9 | SNP2 (S1) | SNP11 | Exon9 | SNP2 (S2) | SNP11 | Exon9 | SNP2 (S3) | SNP11 | Exon9 |
| | | (NTC) | (NTC) | | (S1) | (S1) | | (S2) | (S2) | | (S3) | (S3) |

### SNP Positive Control

23 different double-stranded DNA oligos (gBlocks with custom sequences that are synthesized and bought from Integrated DNA Technologies) were mixed and titrated to provide a single SNP PC that can be used to test the performance and stability of all SNP assays.

### CNV Positive Control

A commercially available genomic DNA was tested and verified to be able to act as the in-plate copy number normalization control.

Features of the kit include:
1. Primer and probes are mixed together and distributed into 8-well strip tubes which come in 3 sets per PCR run
2. Each kit is sufficient to run 30 plates
3. Each plate can be used for 3 samples+1 NTC
4. Each kit will include 2 positive control plates to perform QC of the kit batch before running patient samples
5. Uniform cycling condition for the run as follows (Table 8):

TABLE 8

| | No. of | Temp. | | Analysis |
|---|---|---|---|---|
| Step | cycles | (° C.) | Duration | channel |
| Initial heat | 1 | 95 | 10:00 | N/A |
| activation | | | | |
| Denaturation | 50 | 95 | 0:15 | N/A |
| Combined | | 60 | 1:00 | FAM, HEX |
| annealing/extension | | | | and Cy5 |

FIG. 2 provides the results of the assays carried out.

PCR cycling conditions such as the temperature and duration for the denaturation, annealing and extension steps may be varied depending on factors such as the length and structure of DNA templates, Tm of primers, type of polymerase used, and the relative concentrations of the components of the PCR master mix.

As such, PCR cycling conditions for different reactions can vary greatly, often requiring separate PCR runs for the amplification of different genes. Using PCR for the genotyping of variants of a gene adds a further level of complexity to the design of PCR cycling conditions as further adjustments would be required in order to discriminate between wild-type and mutant alleles.

Advantageously, the method and kit of the present invention is able to produce accurate genotyping of 20 variants in 4 different genes in a single real-time PCR run having a single set of cycling conditions, as evidenced by high degree of variant-level concordance against benchmark methods illustrated in Example 2.

### EXAMPLE 1

The following is a non-limiting example of carrying out the Nala PGx Core™ Kit.

Nala PGx Core™ Kit provides a panel of qualitative tests for 20 variants in 4 genes (CYP2D6, CYP2C9, CYP2C19, and SLCO1B1) on the basis of real-time PCR genotyping. These genes are related to multiple drugs commonly prescribed in the outpatient setting, including cardiovascular, psychiatry, gout medications as well as pain killers. The test is designed to be run in a 96-well plate format on a qPCR platform. Each plate may accommodate up to 3 samples and a no template control.

This panel only requires 48 ng of total DNA input per sample to detect all of the 20 variants.

The identification of patients' genotypes can help physicians deliver a more targeted therapy and reduce trial and error of prescription.

### Kit Components

The following Table 9 sets out the various components of the Nala PGx Core™ Kit.

TABLE 9

| | | Volume | | | |
|---|---|---|---|---|---|
| | Tube | per well | | | Colour |
| Component Name | Name | (μl) | Qty | Format | Code |
| Primer-Probe Mix Set A | PPM_A | 30.0 | 32 | Strip tube | Red |
| Primer-Probe Mix Set B | PPM_B | 30.0 | 32 | Strip tube | Blue |
| Primer-Probe Mix Set C | PPM_C | 30.0 | 32 | Strip tube | Green |
| SNP Positive Control | SNP_PC | 1500 | 1 | Micro-tube | Clear |
| CNV Positive Control | CNV_PC | 440 | 1 | Micro-tube | Clear |
| Master Mix | MM | 5000 | 6 | Bottle | NA |

All reagents apart from the CNV Positive Control must be stored at a temperature between −15° C. to −25° C. The CNV Positive Control should be stored at a temperature between 2° C. to 8° C.

### Method for Carrying Out Assay

### 1. DNA Sample Preparation

1. Genomic DNA should be extracted from samples prior to qPCR set up.
2. Accurately quantify DNA and dilute DNA concentration to 2 ng/μl for use. For each well, 2 μl of template will be added.
3. To ease sample handling, it is recommended that the DNA sample be placed into an 8-well PCR strip-tube with a volume of at least 10 μl per well. Samples can be plated with a multichannel pipette during qPCR set-up. If needed, ensure that strip-tubes are spun down so that reagent loading is accurate.
   2. qPCR Set-Up

### 2.1 Loading of Master Mix

1. Prepare a Bio-Rad Hard-Shell® 96-well run plate
2. Load 8.5 μl of MM into each well of the run plate
3. To ease this process, consider loading an 8-well strip-tube with at least 115 μl of MM in each tube. Perform this step carefully as the MM has a propensity to form bubbles that are not easy to remove later. If needed, ensure that strip-tubes are spun down so that reagent loading is accurate.

### 2.2 Reaction Mix Set Up

1. Gently mix and spin down PPM_A, PPM_B and PPM_C
2. Carefully remove the cap from the strip tubes, taking care not to allow the reagents to flick out.
3. Add 6.5 μl of each PPM into the PCR plate using a multi-channel pipette by following the layout on FIG. 3A (PC plate) or FIG. 3B (Sample plate).
4. Note the orientation of the strip tubes: the wells that are marked should be orientated to the top, and the markings on PPM_A, PPM_B and PPM_C should form a diagonal pattern (see FIG. 3A and FIG. 3B). The orientation is important because each well has a different assay mix inside. The position of the marking on the tube should help to orientate which is the left, centre and right PPM (column-wise) for each sample.

### 2.3 Adding DNA Template

### 2.3.1 For Positive Control Run

1. Following the layout on FIG. 3A, add 2 μl of CNV_PC into wells D3, E3, G3 and H3. Add 2 μl of nuclease-free water into remaining wells of columns 1 to 3.
2. To ease transfer of the remaining SNP_PC and CNV_PC into the plate, prepare the SNP_PC and CNV_PC into an 8-well strip-tube format.
3. Use a multi-channel pipette to transfer 2 μl of the positive controls to columns 4 to 12.
4. Seal plate with optical seal. Do not vortex or flick the plate.
5. Spin down the plate at 1300-2000 rpm for 1 minute. If there are remaining bubbles on the base of the wells, gently tap the base of the plate to try and dislodge the bubbles, and spin the plate once again at 1300-2000 rpm for 1 minute.
6. Proceed to the section for “qPCR Cycling”.

### 2.3.2. For Sample Run

1. Following the layout on FIG. 3B, add 2 μl of CNV_PC into wells D3, E3, G3 and H3. Add 2 μl of nuclease-free water into remaining wells of columns 1 to 3.
2. Add 2 μl of samples into wells in columns 4 to 12 whereby columns 4 to 6 are for sample 1, columns 7 to 9 are for sample 2, columns 10 to 12 are for sample 3.
3. Seal plate with optical seal. Do not vortex or flick the plate.
4. Spin down the plate at 1300-2000 rpm for 1 minute. If there are remaining bubbles on the base of the wells, gently tap the base of the plate to try and dislodge the bubbles, and spin the plate once again at 1300-2000 rpm for 1 minute.
5. Proceed to the section for “qPCR Cycling”.

### 2.4 QPCR Cycling

1. Program the real-time cycler according to the program outlined in Table 10. Sample volume is 17 μl.

TABLE 10
qPCR Cycling Condition for Nala ™ PGx Core

| | No. of | Temp. | | Analysis |
|---|---|---|---|---|
| Step | cycles | (° C.) | Duration | channel |
| Initial heat | 1 | 95 | 10:00 | N/A |
| activation | | | | |
| Denaturation | 50 | 95 | 0:15 | N/A |
| Combined | | 60 | 1:00 | FAM, HEX |
| annealing/extension | | | | and Cy5 |

2. Alternatively while creating the ‘Run Setup’ on CFX96, perform the following to automatically load qPCR cycling protocol and sample plate layout:
   a. click ‘Select Existing’ under ‘Protocol’ tab and load ‘NPGxC_Protocol_TEMPLATE.prcl’ file
   b. click ‘Select Existing’ under ‘Plate’ tab and load ‘NPGxC_PCRun_TEMPLATE.pltd’ file for a Positive Control Run OR ‘NPGxC_SampleRun_TEMPLATE.pltd’ file for an actual Sample Run
3. Place the PCR plate in the real-time cycler, and start the cycling program. Total run time is 95 minutes.
4. For a PC run, save run file (*.pcrd) under this naming format: ‘[YYYYMMDD]_PC[RUN_NUMBER]_MDC_NPGxC.pcrd’, e.g. 20190101_PC001_MDC_NPGxC.pcrd
5. For a Sample run, save run file as ‘[YYYYMMDD]_[RUN_NUMBER]_MDC_NPGxC.pcrd’, e.g. 20190101_001_MDC_NPGxC.pcrd
   3. Data Exporting from Bio-Rad CFX Manager

Assays have been designed for the detection of the variants on Channel 1—FAM (for wild-type alleles), Channel 2—HEX (for mutant alleles), Channel 4—Cy5 (for tri-allele detection of SNP rs5030865 in CYP2D6).

3.1. Change Sample ID (only for Sample plates)
1. Open your .pcrd run file
2. Click ‘Plate Setup’->View/Edit Plate
3. A ‘Plate Editor’ window will pop up. Highlight the sample columns for ‘Sample 1’ (i.e. columns 4 to 6) and change ‘Sample Name’ into the correct ‘Lab Accession ID’. You may connect a barcode scanner to ease this task. Continue on to the next 3 columns until the whole plate is annotated properly. Columns 1 to 3 should be left as is.
4. Click OK to save changes.

### 3.2 Setting of Baseline

1. To set Base preform the following
a. Click Settings->Baseline Setting->Tick both ‘Baseline Subtracted Curve Fit’ and ‘Apply Fluorescence Drift Correction’

### 3.3 Setting of Baseline Start and Baseline End

1. Click on the Quantification tab
2 Deselect display for all channels under the amplification curves until only FAM channel remains
3. Click Settings->Baseline Threshold
4. Under “Baseline Cycles”, select “User Defined”. Click on the top left box of the table below (to select all the wells), and change the “End:” value to 20, and “Begin:” value to 10. NOTE: Always set the End value before setting the Begin value, as the settings will not be consistent if the values are input in the reverse order.
5. Under “Single Threshold”, select “User Defined” and change the threshold value to 300.
6. Click “OK” to save values
7. Repeat steps 2 to 7 for the HEX and Cy5 channels

### 3.4 Export Results

### 3.4.1 Export RFU Values of Each Target (for Plotting Purposes)

1. On ‘Quantification Data’ tab select ‘RFU’
2. Right click on ‘FAM’ tab->click ‘Export to CSV’
3. Do the same for ‘HEX’ and ‘Cy5’ tabs which will result in a total of 3 CSV files differentiated by the last 3 characters of their filenames before the file extension .csv (e.g. ‘[YYYYMMDD]_[RUN NUMBER]_MDC_NPGxC_FAM.csv’).

### 3.4.2 Custom Export of Run Data

1. Click Export->Custom Export
2. Select Export Format as ‘CSV (*.csv)’
3. Tick ‘Include Run Information Header’
4. Under ‘Sample Description’ section select ‘Well’, ‘Fluorophore’, and ‘Sample Name’ only
5. Select ‘Cq’ only under ‘Quantification’ section
6. Select ‘End RFU’ only under ‘End Point’ section
7. Do not select any boxes under Melt Curve
8. Click Export and save file with filename format: ‘[YYYYMMDD]_[RUN NUMBER]_MDC_NPGxC.csv’.
   3.4.3 Annotation and Report Generation through Nalagenetics' Lab Portal

The “Nala Clinical Decision Support™-Lab Manager User Manual” contains further instructions on the steps required for accurate report generation.

Whilst there has been described in the foregoing description preferred embodiments of the present invention, it will be understood by those skilled in the technology concerned that many variations or modifications in details of design or construction may be made without departing from the present invention.

### EXAMPLE 2

The performance of the Nala PGx Core® kit has been validated against established benchmark genotyping methods such as the VeriDose® Core and CYP2D6 Copy Number Variation (CNV) Panel from Agena Bioscience® and TaqMan® DME Genotyping Assays. The validation process and results are described in Kothary et al., 2021.

### Methods and Materials

### Study Recruitment

Participants from the general population were recruited on behalf of Nalagenetics Pte. Ltd. with written informed consent forms from recruitment sites in Singapore and Indonesia, with a minimum of 30 per major ethnic groups residing in both countries—Chinese, Malays, Indians, Caucasians and Indonesians. A total of 251 samples were evaluated from the five major ethnic groups to ensure objective representation amongst the target geographical population. Participants identifying as one or more of the following ethnicities were categorized as Indonesians: Ambon, Batak, Betawi, Jawa, Lampung, Manado, Minangkabau, Nusa Tenggara Timur, Palembang, Sulawesi, Sunda, Timor Leste, Tolaki and Toraja.

Buccal samples were collected using OraCollect (Cat No. DNA OCR-100 from DNA Genotek) and genomic DNA (gDNA) extracted using the Monarch® Genomic DNA Purification Kit (Cat No. T3010 from NEB). The extraction procedure followed manufacturer's instructions with additional dry-spin step at maximum speed for 1 minute after the 2^{nd} buffer washing step. The quality and concentration of gDNA extracts were quantified by NanoDrop 2000 Spectrophotometer (Singapore) and BioDrop-pLITE (Indonesia). The acceptance criteria of DNA quality was as specified in the extraction kit's manufacturer's instruction, i.e. absorbance ratios A260/230 and A260/280 >1.7, and DNA yield >500 ng. Samples that failed to meet the DNA quality control criteria (n=5) were excluded from the study. The remaining extracted gDNA samples (n=246) were stored at −20° C. for downstream application.

### Nala PGx Core®

Nala PGx Core® kit from Nalagenetics Pte. Ltd. consists of 20 qPCR-based variant assays across four genes—CYP2C9, CYP2C19, CYP2D6 and SLCO1B1. The variant assays included in Nala PGx Core® panel of detected alleles were selected based on the following factors in sequential order:
1. Genes with available clinical annotations not lower than level 2B on the PharmGKB criteria for levels of evidence.
2. Clinical annotations were supported by expert consortia (CPIC, DPWG, CPNDS) and regulatory bodies (FDA, PMDA, Swissmedic and EMA).
3. Minor Allele Frequency, MAF >1% for the major ethnic groups residing in the target geographical population.

Whilst assays for CYP2C9, CYP2C19 and CYP2D6 have been designed to enable the detection of specific star alleles, the SLCO1B1 assay has been designed to detect the variant rs4149056, which is present in three reduced function haplotypes namely, SLCO1B1*5, SLCO1B1*15 and SLCO1B1*17. The SLCO1B1 assay is thus, unable to differentiate between each of the three aforementioned haplotypes. The variants covered by the kit are outlined in Table 11.

TABLE 11
Genes and variants evaluated

| | | | | | | Genotyping Methods Utilized | | |
|---|---|---|---|---|---|---|---|---|
| | | | | | | | Agena | |
| | | | | | Allele | | VeriDose ® | TagMan ® |
| | | | | | Clinical | | Core and | DME |
| | Star | | Nucleotide | | Function | | CYP2D6 CNV | Genotyping |
| Gene | Allele | Variant | Changes | Effect On Protein | Status | Nala PGx Core ® | Panel | Assays |
| CYP2C9 | *2 | rs1799853 | 3608C > T | R144C | Decreased | Bi-allelic Assay | VeriDose Core | NA |
| | *3 | rs1057910 | 42614A > C | I359L | Decreased | Bi-allelic Assay | VeriDose Core | NA |
| CYP2C19 | *2 | rs4244285 | 19154G > A | Splicing Defect | None | Bi-allelic Assay | VeriDose Core | NA |
| | *3 | rs4986893 | 17948G > A | W212X | None | Bi-allelic Assay | VeriDose Core | NA |
| | *17 | rs12248560 | −806C > T | 5′ Region | Increased | Bi-allelic Assay | VeriDose Core | NA |
| CYP2D6 | *2 | rs16947, | 2851C > T, | R296C, S486T | Normal | Bi-allelic Assay | VeriDose Core | NA |
| | | rs1135840 | 4181G > C | | | | | |
| | *3 | rs35742686 | 2550delA | Frameshift | None | Bi-allelic Assay | VeriDose Core | NA |
| | *4 | rs3892097, | 1847G > A, | Splicing Defect, P34S | None | Bi-allelic Assay | VeriDose Core | NA |
| | | rs1065852 | 100C > T | | | | | |
| | *5 | N/A | N/A | Gene Deletion | None | CNV Assay (Intron 2) | CYP2D6 CNV | NA |
| | *6 | rs5030655 | 1708delT | Frameshift | None | Bi-allelic Assay | VeriDose Core | NA |
| | *8 | rs5030865 | 1759G > T | G169X | None | Tri-allelic Assay | VeriDose Core | NA |
| | *9 | rs5030656 | 2616delAAG | K281del | Decreased | Bi-allelic Assay | VeriDose Core | NA |
| | *10 | rs3892097, | 1847G > A, | Splicing Defect, P34S | Decreased | Bi-allelic Assay | VeriDose Core | NA |
| | | rs1065852 | 100C > T | | | | | |
| | *14 | rs5030865 | 1759G > A | G169R | None | Tri-allelic Assay | VeriDose Core | NA |
| | *21 | rs72549352 | 2580_2581ins C | Frameshift | None | Bi-allelic Assay | VeriDose Core | NA |
| | *29 | rs59421388 | 3184G > A | V338M | Decreased | Bi-allelic Assay | VeriDose Core | NA |
| | *31 | rs267608319 | 4043G > A | R440H | None | Bi-allelic Assay | NA | AH21B9N |
| | *35 | rs769258 | 31G > A | V11M | Normal | Bi-allelic Assay | NA | C_27102444_F0 |
| | *36 | N/A | Recombination | CYP2D6- | None | CNV Assay (Exon 9) | CYP2D6 CNV | NA |
| | | | at Exon 9 | 2D7 Hybrid | | | | |
| | *41 | rs28371725 | 2989G > A | Splicing Defect | Decreased | Bi-allelic Assay | VeriDose Core | NA |
| SLCO1B1 | NA^{†} | rs4149056 | g.52422T > C | V174A | Decreased | Bi-allelic Assay | VeriDose Core | NA |

^{†}Nala PGx Core ™ detects the variant, rs4149056, which is associated with decreased enzymatic activity and is present in three known SLCO1B1 haplotypes namely, SLCO1B1*5, SLCO1B1*15 and SLCO1B1*17.

Assays were set up on a 96-well plate. Human gDNA was added at a concentration of 2 ng/μL as template for the qPCR reaction, which was then performed on the Bio-Rad CFX96 IVD Touch™ Real-Time PCR Detection System per the product insert. Run analysis was performed using the application CFX Manager 3.1 or CFX Maestro, and exported as raw .csv files. Exported files were uploaded into the companion software, Nala Clinical Decision Support™ (Nala CDS™) for further analysis of variant genotyping, diplotype determination and phenotype translation. The resulting clinical recommendations derived by the software were replicated from their annotations in CPIC, DPWG, or CPNDS, prioritized in sequential order according to their availability from the three databases. Genotyping using Nala PGx Core® was performed at the Molecular Diagnosis Centre, National University Health System, Singapore (NUHS MDC) and PT Nalagenetik Riset Indonesia.

### Agena VeriDose® Core and CYP2D6 Copy Number Variation (CNV) Panel

The VeriDose® Core and CYP2D6 Copy Number Variation (CNV) Panel from Agena Bioscience® consists of 68 variant assays in 20 genes and 5 CYP2D6 CNV assays, accompanied by a reporting software that automatically analyzes each variation. Genotyping using Agena VeriDose Core and CYP2D6 CNV Panel was performed at the Genome Institute of Singapore. Variants evaluated using this platform are listed in Table 11. The Agena VeriDose® Panel has been utilized by the United States Centers for Disease Control and Prevention (CDC) as part of their Genetic Testing Reference Material (GeT-RM) Coordination Program.

### TaqMan® Drug Metabolism Enzyme (DME) Genotyping Assay

TaqMan® DME Genotyping Assays were utilized in the evaluation of CYP2D6 rs769258 (TaqMan Assay ID AH21B9N) and CYP2D6 rs267608319 (TaqMan Assay ID C__27102444_F0). Assays were set up on a 384-well plate with a sample input of human gDNA at 2 ng/μL. The subsequent PCR reaction was performed on the Applied Biosystems ViiA™ 7 Real-Time PCR System as per the recommended cycling conditions, at the Genome Institute of Singapore. Post-PCR plate read was performed using the companion software, TaqMan® Genotyper™ Software for single nucleotide polymorphisms (SNP) genotyping. Similar to the Agena VeriDose® Panel, TaqMan® DME Genotyping Assays were employed in the characterization of DNA samples as part of the CDC GeT-RM program.

### Robustness

Genotype- and diplotype-level call rates were defined as the percentage of samples that returned a genotype at the variant-level or were assigned a distinct diplotype for the gene of interest, respectively. Failed tests were defined as samples that did not return a genotype and/or diplotype call for the genes evaluated.

$$Call Rates , \% = \frac{Total Sample Size - Failed Tests}{Total Sample Size} \times 100 \%$$

### Precision

Three samples at 3 DNA concentrations were tested across 3 reagent lots on 2 machines. Each test condition was repeated within the same plate for a triplicate. For variant assays that identified SNPs and indels, intra-precision was performed within the same plate, run as triplicates across 47 tests. Inter-precision was assessed from 120 tests performed across plate runs covering the 4 variables—samples, DNA concentration, reagent lots and machines. Concordance rates across precision studies were calculated as the percentage of tests that returned a genotype call concordant to the expected truth for each variant assay. Discordant genotype was defined as instances when the test returned a genotype call that was different from the expected truth.

$$Concordance Rate , \% = \frac{No . Of Tests Performed - No . Of Tests With Discordant Genotype}{No . Of Tests Performed} \times 100 \%$$

For CYP2D6 CNV assays, copy number estimates for Intron 2 and Exon 9 of the three samples were derived based on their cycle threshold (Ct) results across plate runs.

$$Copy Number = 2 \times {2}^{- ΔΔ Ct}$$

$$ΔΔ Ct = ( {Ct}_{reference gene calibrator} - {Ct}_{CYP 2 D 6 calibrator} ) - ( {Ct}_{reference gene} - {Ct}_{CYP 2 D 6 sample} )$$

Testing of the three samples was repeated for a number of plate runs, n, and calculated for the average copy number of each sample and their coefficient of variation (CV). The CV for each plate run was calculated by finding the standard deviation (σₚₗₐₜₑ) between triplicates within the same plate run, and divided by the triplicate mean (μₚₗₐₜₑ). The average of the individual CVs was reported as the intra-precision CV. For inter-precision CV, standard deviation population (σ_{plate means}) was divided by the mean population, i.e. average of means.

$$Intra - CV , \% = \frac{{\sum }_{n} \frac{{σ}_{plate}}{{µ}_{plate}}}{n} \times 100 \%$$

$$Inter - CV , \% = \frac{{σ}_{plate means}}{{\sum }_{n} {µ}_{plate / n}} \times 100 \%$$

### Accuracy

### Variant-level Concordance

The accuracy of Nala PGx Core® in genotyping at a variant-level was evaluated by comparing calls produced by Nala PGx Core® assay against benchmark methods as listed in Table 11. Samples that successfully produced genotype calls for all variants tested on Nala PGx Core® and its benchmarks were considered for the evaluation (n=225 for all variants except CYP2D6 CNV; n=224 for CYP2D6 CNV). Samples that failed to produce a genotype call on one or more of the platforms were excluded from the concordance calculation (n=21/225 for all variants except CYP2D6CNV; n=22/224 for CYP2D6CNV). Discordant calls were defined as instances in which Nala PGx Core® provided a genotype call that was different from that of a call made by the corresponding benchmark. Percentage concordance to the benchmark was calculated per variant as follows—

$$Concordance To Benchmark Per Variant , \% = \frac{Total Sample Size - Discordant Calls By Nala PGx {Core}^{®}}{Total Sample Size}$$

### Diplotype-level Concordance

The accuracy of Nala PGx Core® in assigning a diplotype call for CYP2C9, CYP2C19, and CYP2D6, was evaluated by comparing calls against the Agena VeriDose® Core and CYP2D6 CNV Panel. Samples that met the following criteria were included in the sample size of each gene:
1. Successful genotype-level calls on the relevant platforms for all variants covered by the gene of interest
2. Successful assignment of a diplotype for the gene of interest on both Nala PGx Core®, and Agena VeriDose® Core and CYP2D6 CNV Panel

Discordant calls were defined as instances in which Nala PGx Core® assigned a diplotype that differed from the call made by the Agena VeriDose® Core and CYP2D6 CNV Panel.

$$Concordance , \% = \frac{Total Sample Size - Discordant Calls By Nala PGx {Core}^{®}}{Total Sample Size} \times 100 \%$$

### Frequencies by Ethnicity

Ethnicities were obtained based on participant self-identification across both the population cohorts as part of the recruitment questionnaire. Out of 251 participants, the following were excluded from the frequency analysis:
1. Samples in which participants did not report an ethnic group on the recruitment form (n=6)
2. Samples with one or more variant level failures across the 4 genes evaluated in Table 11 (n=18)
3. Samples with one or more diplotype-level failures (“No Call”) for the gene of interest on Nala PGx Core®, Agena VeriDose® Core and CYP2D6 CNV Panel or both (n=variable)
4. Samples with discordant diplotype calls for the gene of interest (n=variable)

The remaining samples were included in the allele-level frequency analysis of CYP2C9 (n=206), CYP2C19 (n=201), CYP2D6 (n=195) and SLCO1B1 (n=203), as well as in the diplotype-level frequency analysis of CYP2C9 (n=206), CYP2C19 (n=201) and CYP2D6 (n=195). Allele and diplotype frequency values were derived using the following formulae, for both the overall study cohort as well as for each ethnic group.

$$Frequency of Allele ‘ X ’ In A Given Population = \frac{Total Copies Of Allele ‘ X ’}{Total Copies Of All Alleles For The Gene Of Interest}$$

$$Frequency of Diplotype ‘ X ’ In A Given Population = \frac{Total Instances Of Diplotype ‘ X ’}{Total Number Of Individuals In The Population}$$

### Results

### Robustness

Evaluation of the observed genotype- and diplotype-level call rates of the platforms evaluated in this study was carried out. 246 samples underwent variant genotyping and diplotype determination, across the four genes evaluated on the genotyping platforms (Tables 12, 13).

The genotype-level call rates for Nala PGx Core® were at 100% for CYP2C9, CYP2C19 and SLCO1B1, and the diplotype-level call rates were at 100% for CYP2C9 and CYP2C19. The benchmark platform, Agena VeriDose® Core Panel, demonstrated call rates of >95.9% at the genotype-level and >90.7% at the diplotype-level.

TABLE 12
Observed genotype-level call rates per variant per gene per platform

| | | Variant Call Rate, % (n = 246) | | |
|---|---|---|---|---|
| | | | | TaqMan ® DME |
| | | Nala PGx | Agena VeriDose ® Core | Genotyping |
| Gene | Variant | Core ® | and CYP2D6 CNV Panel | Assays |
| CYP2C9 | rs1799853 | 100.0 | 99.2 | NA |
| | rs1057910 | 100.0 | 99.6 | NA |
| CYP2C19 | rs4244285 | 100.0 | 99.6 | NA |
| | rs4986893 | 100.0 | 99.6 | NA |
| | rs12248560 | 100.0 | 98.8 | NA |
| CYP2D6 | rs1065852 | 98.4 | 95.9 | NA |
| | rs5030655 | 100.0 | 99.2 | NA |
| | rs3892097 | 98.8 | 99.2 | NA |
| | rs35742686 | 100.0 | 98.8 | NA |
| | rs16947 | 100.0 | 99.6 | NA |
| | rs28371725 | 100.0 | 99.2 | NA |
| | rs1135840 | 100.0 | 99.6 | NA |
| | rs769258 | 98.8 | NA | 100.0 |
| | rs5030865 | 97.2 | 99.2 | NA |
| | rs5030656 | 100.0 | 99.6 | NA |
| | rs59421388 | 100.0 | 99.2 | NA |
| | rs267608319 | 99.6 | NA | 100.0 |
| | CNV Assay | 99.6 | 99.2 | NA |
| | (Intron 2) | | | |
| | CNV Assay | 99.6 | 99.2 | NA |
| | (Exon 9) | | | |
| SLCO1B1 | rs4149056 | 100.0 | 99.2 | NA |

TABLE 13
Observed diplotype-level call rates per gene per platform

Diplotype Call Rate, % (n = 246)

| | | Agena VeriDose ® Core |
|---|---|---|
| Gene | Nala PGx Core ® | and CYP2D6 CNV Panel |
| CYP2C9 | 100.0 | 97.2 |
| CYP2C19 | 100.0 | 98.8 |
| CYP2D6 | 95.9 | 90.7 |

Most variants in CYP2D6, except for seven, achieved 100% call rates on Nala PGx Core®, while the corresponding call rates of the benchmark platforms were observed to be between 95.9-99.2% on Agena VeriDose® Core and CYP2D6 CNV Panel, and 100% on TaqMan® DME Genotyping Assays. Out of the seven aforementioned variants, Nala PGx Core® demonstrated higher call rates than the benchmark for the genotyping of rs1065852, Intron 2 and Exon 9 variants. For rs3892097, rs769258, rs5030865, and rs267608319, the accompanying benchmarks demonstrated higher call rates. At the diplotype-level, Nala PGx Core® demonstrated a CYP2D6 call rate of 95.9% as compared to the benchmark, which was observed to be at 90.7% (Table 13).

### Precision

A precision study was conducted to assess the consistency of Nala PGx Core® for samples tested under the same conditions (intra-precision) and under different conditions (inter-precision). Both study resulted in 100% concordance for all assays across replicates, demonstrating consistent genotyping results across a range of DNA concentration, reagent lots and machine variations. Precision of CYP2D6 CNV assay was reported as the average copy number obtained for Intron 2 and Exon 9 of three samples, and their CV calculated across the test conditions. The intra-CV ranged from 3-6% while inter-CV between 5-13%, demonstrating high precision of the assays across variables, where acceptable ranges were intra-CV below 10% and inter-CV below 15%.

### Accuracy

### Variant-Level Concordance

To assess the accuracy of the panel, 20 variant assays comprising of 18 SNPs and 2 CYP2D6 Copy Number assays were genotyped on the panel, Nala PGx Core®, against benchmark methods as listed in Table 11. The 225 sample cohort consisted of DNA samples isolated from buccal swabs that had successfully produced genotype calls for all variants tested on Nala PGx Core® and its benchmarks.

11 variants (CYP2C9 rs1799853, rs1057910; CYP2C19 rs12248560; CYP2D6 rs5030655, rs3892097, rs35742686, rs28371725, rs769258, rs5030656, rs59421388, rs267608319) were genotyped against Agena VeriDose® Core with a resulting concordance rate of 100% (N=225 samples). Discordance was observed for CYP2C19 rs4244285 (n=7) and rs4986893 (n=3), resulting in misidentification of *2 and *3 star alleles. For CYP2D6, discordant genotyping at rs1065852 (n=1), rs16947 (n=7), rs1135840 (n=5) and rs5030865 (n=1) caused misidentification of *2, *4, *8, *10 and *14 star alleles. Variant discordance was also observed at SLCO1B1 rs4149056 (n=6), where Nala PGx Core either detected the presence of SNP on a chromatid that the benchmark did not (n=2), or did not detect a SNP chromatid that was present on the benchmark (n=4). Altogether, this resulted in a mismatch rate of 0.44% to 3.1% for the affected assays. Overall, Nala PGx Core® demonstrated >96% concordance to the benchmark, Agena VeriDose® Core, for the 16 variants across 225 samples.

Variants not present on Agena VeriDose® Core, CYP2D6 rs769258 and CYP2D6 rs267608319, were genotyped using TaqMan® DME Genotyping Assays. Nala PGx Core® demonstrated 100% concordance (N=225) to the benchmark for both SNPs.

For the CYP2D6 Intron 2 and Exon 9 Copy Number assays, concordance was observed to be at 99.6% and 98.7% respectively, against the Agena CYP2D6 CNV Panel. Discordant calls were observed in samples with an Intron 2 copy number greater than 3 (n=1), and for samples with an Exon 9 copy number of one (n=1) and two (n=2).

### Diplotype-Level Concordance

Following successful genotyping at the variant level, the accuracy of Nala PGx Core® in assigning a diplotype call for CYP2C9, CYP2C19 and CYP2D6 was investigated, with reference to the Agena VeriDose® Core and CYP2D6 CNV Panel. Table 14 displays the percentage concordance after the further exclusion of samples that demonstrated diplotype mismatches arising from technological differences, where technological differences refer to the varying allele coverage of each platform. These differences were derived from the variant lists of both Nala PGx Core® (Table 11) and its benchmark, the Agena VeriDose® Core and CYP2D6 CNV Panel.

TABLE 14
Diplotype concordance for CYP2C9, CYP2C19 and CYP2D6
between Nala PGx Core ®, and Agena VeriDose ® Core
and CYP2D6 CNV Panel

| | | | Discordant Diplotypes | | |
|---|---|---|---|---|---|
| | | | Agena VeriDose ® | | |
| | | | Core and CYP2D6 | | |
| Genes | Concordance, %^{†} | | CNV Panel | Nala PGx Core ® | Instances |
| CYP2C9 | 100% | (n = 221) | NA | NA | NA |
| CYP2C19 | 96.4% | (n = 223) | *1/*1 | *1/*3 | 1 |
| | | | *1/*2 | *1/*1 | 1 |
| | | | *1/*2 | *1/*3 | 1 |
| | | | *1/*3 | *2/*2 | 1 |
| | | | *2/*2 | *1/*1 | 2 |
| | | | *2/*2 | *1/*2 | 2 |
| CYP2D6 | 94.7% | (n = 209) | *1/*1 | *2/*2 | 1 |

| | | |
|---|---|---|
| *1/*10 | *1/*10, CN >= 3 | 1 |
| *1/*41 | *39/*41 | 1 |
| *2/*2 | *1/*1 | 1 |
| *2/*10 | *1/*10 | 1 |
| *2 × N/*36 × N, | *2/*10, CN >= 3 | 1 |
| CN >= 3 | | |
| *4 × 2/*36 × N, | *4/*10, CN >= 3 | 2 |
| CN >= 3 | | |
| *10 × 2/*36 × N, | *2 × 2/*36 × N, | 1 |
| CN >= 3 | CN >= 3 | |
| *13 *1/*10, | *1/*10, CN >= 3 | 1 |
| CN >= 3 | | |
| *13 *1/*41 | *1/*41 | 1 |

^{†}The concordance presented in this table excludes samples that have mismatches in diplotype calls arising from technological differences between platforms.

Overall, a percentage agreement of 100% for CYP2C9 (n=221), 96.4% for CYPC219 (n=223) and 94.7% for CYP2D6 (n=209) was observed between Nala PGx Core® and the benchmark. Discordance was observed at n=1 for all diplotypes listed in Table 14 except for the following with more than one discordant calls: CYP2C19 *2/*2 (n=4), and CYP2D6 *4×2/*36×N, CN>=3 (n=2).

### Frequencies by Ethnicity

For samples that were concordant on Nala PGx Core® and the benchmark platforms, the allele frequencies amongst the populations residing in Singapore and Indonesia (Table 15) were able to be observed. From the combination of alleles present in individual's chromosome, both the diplotype and corresponding phenotype frequencies amongst our study population were able to be observed (Table 16).

TABLE 15
Observed allele frequencies by ethnicity

| | | | | | | | | Allele Frequencies | | |
|---|---|---|---|---|---|---|---|---|---|---|
| | | | | | | | | (PharmGKB) | | |
| | Allele or | Allele Frequencies (Per This Study) | | | | | | East | Central/ | |
| Gene | Variant | Indonesian | Chinese | Malay | Indian | Caucasian | Overall | Asian | South Asian | European |
| CYP2C9 | *2 | 0.000 | 0.000 | 0.000 | 0.060 | 0.172 | 0.032 | 0.002 | 0.114 | 0.127 |
| (n = 206) | *3 | 0.000 | 0.040 | 0.039 | 0.100 | 0.069 | 0.044 | 0.038 | 0.110 | 0.076 |
| CYP2C19 | *2 | 0.297 | 0.274 | 0.289 | 0.229 | 0.138 | 0.256 | 0.284 | 0.270 | 0.147 |
| (n = 201) | *3 | 0.041 | 0.055 | 0.066 | 0.000 | 0.017 | 0.042 | 0.072 | 0.016 | 0.002 |
| | *17 | 0.054 | 0.007 | 0.039 | 0.229 | 0.138 | 0.067 | 0.021 | 0.171 | 0.216 |
| CYP2D6 | *2 | 0.088 | 0.103 | 0.118 | 0.250 | 0.212 | 0.136 | 0.121 | 0.295 | 0.277 |
| (n = 195) | *4 | 0.000 | 0.007 | 0.026 | 0.083 | 0.250 | 0.051 | 0.005 | 0.091 | 0.185 |
| | *5 | 0.029 | 0.027 | 0.053 | 0.021 | 0.019 | 0.031 | 0.049 | 0.046 | 0.030 |
| | *6 | 0.000 | 0.000 | 0.000 | 0.000 | 0.019 | 0.003 | 0.000 | 0.000 | 0.011 |
| | *9 | 0.000 | 0.000 | 0.000 | 0.000 | 0.019 | 0.003 | 0.002 | 0.003 | 0.028 |
| | *10 | 0.338 | 0.349 | 0.250 | 0.104 | 0.000 | 0.251 | 0.436 | 0.087 | 0.016 |
| | *14 | 0.000 | 0.014 | 0.000 | 0.000 | 0.000 | 0.005 | 0.003 | ND | 0.000 |
| | *29 | 0.000 | 0.000 | 0.000 | 0.000 | 0.019 | 0.003 | 0.000 | 0.003 | 0.001 |
| | *36 | 0.206 | 0.281 | 0.211 | 0.063 | 0.000 | 0.190 | 0.012 | 0.000 | 0.000 |
| | *41 | 0.044 | 0.041 | 0.026 | 0.104 | 0.058 | 0.049 | 0.023 | 0.123 | 0.092 |
| SLCO1B1 | rs4149056^{†} | 0.125 | 0.074 | 0.026 | 0.040 | 0.167 | 0.084 | 0.125^{‡} | 0.050^{‡} | 0.159^{‡} |
| (n = 203) | | | | | | | | | | |

“ND” refers to instances in which no data is available for the given allele on PharmGKB.
^{†}rs4149056 refers to the reduced function variant of SLCO1B1 that is present in SLCO1B1*5, SLCO1B1*15 and SLCO1B1*17.
^{‡}Allele frequency values for rs4149056 have been obtained from gnomAD.

TABLE 16
Observed diplotype frequencies by ethnicity

| | | | Diplotype Frequencies (Per This Study)^{†} | | | | | | | | | | | |
|---|---|---|---|---|---|---|---|---|---|---|---|---|---|---|
| | | | Indonesian | | Chinese | | Malay | | Indian | | Caucasian | | Overall | |
| Gene | Diplotype | Phenotype ^{‡} | Obs | Freq | Obs | Freq | Obs | Freq | Obs | Freq | Obs | Freq | Obs | Freq |
| CYP2C9 | *1/*1 | NM | 39 | 1.000 | 69 | 0.920 | 35 | 0.921 | 17 | 0.680 | 16 | 0.552 | 176 | 0.854 |
| | *1/*2 | IM | 0 | 0.000 | 0 | 0.000 | 0 | 0.000 | 3 | 0.120 | 9 | 0.310 | 12 | 0.058 |
| | *1/*3 | IM | 0 | 0.000 | 6 | 0.080 | 3 | 0.079 | 5 | 0.200 | 3 | 0.103 | 17 | 0.083 |
| | *2/*3 | PM | 0 | 0.000 | 0 | 0.000 | 0 | 0.000 | 0 | 0.000 | 1 | 0.034 | 1 | 0.005 |
| CYP2C19 | *1/*1 | NM | 10 | 0.270 | 31 | 0.425 | 14 | 0.368 | 8 | 0.333 | 14 | 0.483 | 77 | 0.383 |
| | *1/*2 | IM | 19 | 0.514 | 28 | 0.384 | 14 | 0.368 | 5 | 0.208 | 7 | 0.241 | 73 | 0.363 |
| | *1/*3 | IM | 3 | 0.081 | 6 | 0.082 | 3 | 0.079 | 0 | 0.000 | 1 | 0.034 | 13 | 0.065 |
| | *1/*17 | RM | 3 | 0.081 | 1 | 0.014 | 1 | 0.026 | 5 | 0.208 | 5 | 0.172 | 15 | 0.075 |
| | *2/*2 | PM | 1 | 0.027 | 5 | 0.068 | 3 | 0.079 | 1 | 0.042 | 0 | 0.000 | 10 | 0.050 |
| | *2/*3 | PM | 0 | 0.000 | 2 | 0.027 | 2 | 0.053 | 0 | 0.000 | 0 | 0.000 | 4 | 0.020 |
| | *2/*17 | IM | 1 | 0.027 | 0 | 0.000 | 0 | 0.000 | 4 | 0.167 | 1 | 0.034 | 6 | 0.030 |
| | *17/*17 | UM | 0 | 0.000 | 0 | 0.000 | 1 | 0.026 | 1 | 0.042 | 1 | 0.034 | 3 | 0.015 |
| CYP2D6 | *1/*1 | NM | 6 | 0.176 | 4 | 0.055 | 6 | 0.158 | 4 | 0.167 | 4 | 0.154 | 24 | 0.123 |
| | *1/*2 | NM | 0 | 0.000 | 3 | 0.041 | 1 | 0.026 | 4 | 0.167 | 2 | 0.077 | 10 | 0.051 |
| | *1/*2, | UM | 0 | 0.000 | 0 | 0.000 | 0 | 0.000 | 0 | 0.000 | 2 | 0.077 | 2 | 0.010 |
| | CN >= 3 | | | | | | | | | | | | | |
| | *1/*4 | IM | 0 | 0.000 | 1 | 0.014 | 1 | 0.026 | 1 | 0.042 | 4 | 0.154 | 7 | 0.036 |
| | *1/*5 | IM | 0 | 0.000 | 2 | 0.027 | 1 | 0.026 | 0 | 0.000 | 1 | 0.038 | 4 | 0.021 |
| | *1/*6 | IM | 0 | 0.000 | 0 | 0.000 | 0 | 0.000 | 0 | 0.000 | 1 | 0.038 | 1 | 0.005 |
| | *1/*10 | NM | 4 | 0.118 | 5 | 0.068 | 3 | 0.079 | 1 | 0.042 | 0 | 0.000 | 13 | 0.067 |
| | *1/*14 | NM | 0 | 0.000 | 1 | 0.014 | 0 | 0.000 | 0 | 0.000 | 0 | 0.000 | 1 | 0.005 |
| | *1/*29 | NM | 0 | 0.000 | 0 | 0.000 | 0 | 0.000 | 0 | 0.000 | 1 | 0.038 | 1 | 0.005 |
| | *1 × 2/*36 × N, | NM | 3 | 0.088 | 4 | 0.055 | 6 | 0.158 | 2 | 0.083 | 0 | 0.000 | 15 | 0.077 |
| | CN >= 3 | | | | | | | | | | | | | |
| | *1/*41 | NM | 1 | 0.029 | 2 | 0.027 | 0 | 0.000 | 1 | 0.042 | 2 | 0.077 | 6 | 0.031 |
| | *1/*41, | NM | 0 | 0.000 | 0 | 0.000 | 0 | 0.000 | 1 | 0.042 | 0 | 0.000 | 1 | 0.005 |
| | CN >= 3 | | | | | | | | | | | | | |
| | *2/*2 | NM | 0 | 0.000 | 1 | 0.014 | 0 | 0.000 | 1 | 0.042 | 2 | 0.077 | 4 | 0.021 |
| | *2/*4 | IM | 0 | 0.000 | 0 | 0.000 | 0 | 0.000 | 1 | 0.042 | 2 | 0.077 | 3 | 0.015 |
| | *2/*5 | IM | 0 | 0.000 | 0 | 0.000 | 2 | 0.053 | 0 | 0.000 | 0 | 0.000 | 2 | 0.010 |
| | *2/*10 | NM | 4 | 0.118 | 4 | 0.055 | 3 | 0.079 | 2 | 0.083 | 0 | 0.000 | 13 | 0.067 |
| | *2/*14 | NM | 0 | 0.000 | 1 | 0.014 | 0 | 0.000 | 0 | 0.000 | 0 | 0.000 | 1 | 0.005 |
| | *2 × 2/*36 × N, | NM | 2 | 0.059 | 3 | 0.041 | 3 | 0.079 | 1 | 0.042 | 0 | 0.000 | 9 | 0.046 |
| | CN >= 3 | | | | | | | | | | | | | |
| | *2/*41 | NM | 0 | 0.000 | 2 | 0.027 | 0 | 0.000 | 2 | 0.083 | 1 | 0.038 | 5 | 0.026 |
| | *4/*4 | PM | 0 | 0.000 | 0 | 0.000 | 0 | 0.000 | 0 | 0.000 | 3 | 0.115 | 3 | 0.015 |
| | *4/*5 | PM | 0 | 0.000 | 0 | 0.000 | 0 | 0.000 | 1 | 0.042 | 0 | 0.000 | 1 | 0.005 |
| | *4/*9 | IM | 0 | 0.000 | 0 | 0.000 | 0 | 0.000 | 0 | 0.000 | 1 | 0.038 | 1 | 0.005 |
| | *4/*10 | IM | 0 | 0.000 | 0 | 0.000 | 1 | 0.026 | 1 | 0.042 | 0 | 0.000 | 2 | 0.010 |
| | *5/*10 | IM | 0 | 0.000 | 2 | 0.027 | 1 | 0.026 | 0 | 0.000 | 0 | 0.000 | 3 | 0.015 |
| | *5/*41 | IM | 2 | 0.059 | 0 | 0.000 | 0 | 0.000 | 0 | 0.000 | 0 | 0.000 | 2 | 0.010 |
| | *10/*10 | IM | 3 | 0.088 | 2 | 0.027 | 3 | 0.079 | 0 | 0.000 | 0 | 0.000 | 8 | 0.041 |
| | *10/*10, | IM | 0 | 0.000 | 1 | 0.014 | 0 | 0.000 | 0 | 0.000 | 0 | 0.000 | 1 | 0.005 |
| | CN >= 3 | | | | | | | | | | | | | |
| | *10/*36 | IM | 1 | 0.029 | 9 | 0.123 | 0 | 0.000 | 0 | 0.000 | 0 | 0.000 | 10 | 0.051 |
| | *10/*36 × N, | IM | 0 | 0.000 | 1 | 0.014 | 0 | 0.000 | 0 | 0.000 | 0 | 0.000 | 1 | 0.005 |
| | CN >= 3 | | | | | | | | | | | | | |
| | *10 × 2/*36 × | IM | 8 | 0.235 | 23 | 0.315 | 5 | 0.132 | 0 | 0.000 | 0 | 0.000 | 36 | 0.185 |
| | N, CN >= 3 | | | | | | | | | | | | | |
| | *10/*41 | IM | 0 | 0.000 | 1 | 0.014 | 0 | 0.000 | 1 | 0.042 | 0 | 0.000 | 2 | 0.010 |
| | *36 × N/*41 × | NM | 0 | 0.000 | 1 | 0.014 | 2 | 0.053 | 0 | 0.000 | 0 | 0.000 | 3 | 0.015 |
| | 2, CN >= 3 | | | | | | | | | | | | | |

^{†}“Obs” and “Freq” are abbreviations for “Observations” and “Frequency” respectively.
^{‡} “NM, “IM, “PM”, “RM” and “UM” are abbreviations for “Normal Metabolizer”, “Intermediate Metabolizer”, “Poor Metabolizer”, “Rapid Metabolizer” and “Ultrarapid Metabolizer” respectively.

For CYP2C9, *3 allele was the most common amongst Chinese and Malay, and *2 allele amongst Caucasian, which is in line with PharmGKB's reported distribution for the East Asian and European populations respectively. Our study also reported *3 allele as the more common variant in Indian population than *2, as opposed to PharmGKB's frequency. These allele frequencies translated to *1/*3 as a common diplotype observed in Chinese, Malay and Indians, and *1/*2 in Caucasians.

For CYP2C19, the highest frequency of CYP2C19*2 was observed amongst Chinese, Malay and Indonesian which were categorized as East Asian populations. This resulted into high frequency of *1/*2 heterozygous depicted as a common diplotype amongst the population. The alleles *2 and *17 were observed as the common variants at equal proportions of 0.229 in Indians and 0.138 in Caucasians. CYP2C19*3 was a common minor allele least observed amongst Indians and Caucasians, 0 and 0.017 respectively. As a result, *1/*2 and *1/*17 were common diplotypes observed in Indian and Caucasian populations, and *2/*17 only seen in Indians.

Common polymorphisms of CYP2D6 in our population were seen in *10 and *36 alleles, at almost three-fold higher frequencies in Chinese, Malay and Indonesian than in Indians. High frequencies of at least one copy of *36 in were noticed our East Asian population. Additionally, 1.4% of the Chinese population who participated in our study carried at least two or more copies of the *36 allele (FIG. 4). These alleles resulted in high frequencies of *10×2/*36×N CN>=3 amongst the Chinese, Malay and Indonesian populations. The alleles with the highest frequency amongst our Indian population included *2, *4, *10 and *41, which were similar to values reported by PharmGKB. Although lower than other ethnic groups, presence of at least one copy of *36 allele at 0.063 frequency amongst Indians was observed, as opposed to none reported in the Central/South Asian population by PharmGKB. The corresponding common diplotypes observed in Indians were *1/*2, *1×2/*36×N CN>=3, *2/*10 and *2/*41 ranging from 0.083 to 0.167 of the cohort. The alleles *2 and *4 were most common amongst Caucasians resulting in high frequency diplotypes of *1/*4 and *4/*4 at 0.154 and 0.115 respectively. Similarly, *1/*2, *1/*2 CN>=3, *2/*2, *2/*4 and *1/*41 were observed in equal proportion at 0.077.

For SLCO1B1, the frequencies of rs4149056 across all ethnicities were consistent with values reported in gnomAD, with the variant being most common amongst Caucasians (0.167) and least amongst Indians (0.040). The frequency amongst East Asians (0.125, gnomAD), as denoted by the Chinese and Indonesian ethnic groups in this study, ranged between 0.074 and 0.125 respectively.

### Discussion

Here, evaluation of the performance of Nala PGx Core®, a qPCR-based panel that evaluates 18 variants and 2 CYP2D6 Copy Number markers across 4 pharmacogenes with established relevance across major ethnic groups in Singapore and Indonesia population was carried out. Nala PGx Core® comes coupled with a reporting software that supports variant detection, diplotype assignment, diplotype-to-phenotype translation and the generation of reports containing clinical recommendations for each phenotype. Altogether, the operation of Nala PGx Core® from receipt of specimen to generation of genotype results could complete within a day. The panel demonstrated high genotype-level call rates of >97% for CYP2D6, and 100% for CYP2C9, CYP2C19 AND SLCO1B1. Similarly, high diplotype-level call rates were observed at >95% for CYP2D6, and 100% for CYP2C9 and CYP2C19. A precision of 100% was observed under the same conditions (intra) and across different conditions (inter). In comparison to other established platforms serving as benchmarks during the study, Nala PGx Core® had ≥96.9% concordance rate for all variant level assays, which consequently resulted in 294.7% concordance at a diplotype level across CYP2C9, CYP2C19 and CYP2D6.

Failures to produce a variant genotype call could be attributed to several reasons. Firstly, failures could potentially stem from the quality of gDNA, despite the DNA quality checks (QC) performed prior to accepting a sample for testing. Poor DNA quality could arise from multiple factors along the sample handling chain. Such factors include the contamination of the buccal fluid by interfering particles during sample collection, inconsistent conditions during sample transport and human error during sample purification. These may lead to the degradation of genomic DNA, poor homogenization of the sample in collection and/or extraction buffers, and the carryover of contaminants, thereby compromising sample integrity. Further QC that involves specific quantification of double-stranded non-fragmented DNA and traces of other interfering materials like RNA, carryover carbohydrate, residual phenol, guanidine or other reagents could enhance the call rate. Regardless, the overall higher variant call rates on Nala PGx Core® panel demonstrate high tolerance of interfering substances, therefore alluding to the high robustness of the assay. Often, failures at variant genotyping subsequently contribute to failures at determining diplotype, since an incomplete variant panel cannot translate into a diplotype. Failures at diplotype calling could also arise from a combination of variants that do not map onto a distinct diplotype, per the reference database, potentially indicating a novel combination.

Next, the allele frequency distribution in the study cohort across the 5 major ethnic groups observed (Indonesian, Chinese, Malay, Indian and Caucasian) was evaluated. The data presented was limited strictly to the geographical boundaries of Singapore and Indonesia, which could account for the difference in allele frequencies observed in comparison to PharmGKB, which is representative of a more expansive and global cohort. Whilst dissimilar to database figures, this invention demonstrated the distributions for the following to be concordant with previous studies, suggesting a niche in the PGx landscape of Singapore and Indonesia—
1. CYP2C9*3 allele as the more common variant within Indians than CYP2C9*2
2. Presence of at least one copy of CYP2D6*36 allele frequency amongst Indians
3. A SLCO1B1 rs4149056 frequency of 12.5% amongst Indonesians
4. High frequencies of the CYP2D6*10 amongst Indonesians and Chinese
5. High frequencies of the CYP2D6*36 allele as seen in the Indonesian, Chinese and Malay ethnicities
6. Two or more copies of CYP2D6*36 within our Chinese population Due to the lack of CYP2D6 copy number references, it is our understanding that the frequency of CYP2D6*36 in Indonesia may not be well-represented. Our study revealed that the prevalence of CYP2D6*36 to be approximately seventeen times higher amongst Indonesians as compared to the corresponding East Asian allele frequency on PharmGKB. Furthermore, our study provides insight on the frequencies of the CYP2D6 *10/*36 diplotype in the archipelago, including those of *10/*36×N CN>=3 and *10×2/*36×N CN>=3, which may help inform the adoption of population-specific PGx workflows regionally. Taken together, the data presents a case for extending tailored PGx testing across the 4 pharmacogenes studied, CYP2C9, CYP2C19, CYP2D6 and SLCO1B1, in South East Asia.

### EXAMPLE 3

In Maggadani et al., 2021, the Nala PGx Core® kit was used for the CYP2D6 genotyping of Indonesian ER+ breast cancer (BC) patients.

Estrogen receptor (ER) expression is the main indicator of potential responses to hormonal therapy, and approximately 70% of human breast cancers are hormone-dependent and ER+. Hormone receptor-positive BC is associated with less aggressive features and a better prognosis because of the benefits from currently available endocrine therapy. Tamoxifen is the current standard of care for ER+ breast cancer adjuvant therapy. It works by binding to the estrogen receptor. The drug has been proven effective in reducing the number of recurrences especially in pre-menopausal women. About 170,000 tamoxifen prescriptions were filed in 2015 in Indonesia, which implies that the usage of this drug has been prevalent in Indonesia to treat ER+ breast cancer.

Tamoxifen is a prodrug that needs to be metabolized to be active. However, half of the patients receiving tamoxifen may not have the full benefit of this drug due to the genetic polymorphisms that affect the function of the main enzyme metabolizing tamoxifen, CYP2D6. Tamoxifen is metabolized to 4-hydroxy-N-desmethyltamoxifen (endoxifen), which has been proven to be an important contributor to the overall anticancer effect. Endoxifen is formed predominantly by CYP2D6 from N-desmethyltamoxifen, the most abundant metabolite. Endoxifen threshold value has been discovered to significantly impact breast cancer survival rates. Upon years of follow up, those with endoxifen levels lower than 5.97 ng/mL had a 30% higher chance of having recurrence of breast cancer. It was further showed that being a CYP2D6 poor/intermediate metabolizer was associated with having a higher Body Mass Index (BMI), and consequently lower tamoxifen concentrations predicted risk for breast cancer recurrence. Additionally, study has also shown that individual variability of CYP2D6 contributed 53% towards the ratio of N-desmethyltamoxifen and endoxifen, while combined other CYPs genetic factors (CYP2C9, CYP2C19, CYP3A5) and non-genetic factors (age, BMI) contributed to only 2.8%.

CYP2D6 gene that encodes Cytochrome P450 2D6 (CYP2D6) enzyme has more than 100 variants; some causing reduced activity, and others causing complete loss of function. The spectrum of the CYP2D6 enzymatic activity translates to different metabolizer profiles that are grouped into normal, ultrarapid, extensive, intermediate, and poor metabolizers (NM, UM, EM, IM, and PM, respectively), depending on how many reducing and/or loss of function alleles an individual carries. Asians and Africans were known to have up to 50% reduced activity alleles. In Malays, Chinese and Indians, intermediate metabolizers occur in 35%, 45.38%, and 15%, respectively. Meanwhile, Caucasians were commonly extensive metabolizers. CYP2D6 ultrarapid and extensive metabolizers are able to take tamoxifen as indicated, according to the guidelines by Clinical Pharmacogenetics Implementation Consortium (CPIC).

This example aims to observe the distribution of CYP2D6 genotypes and its correlation with endoxifen levels in ER+ breast cancer patients in Indonesia. CYP2D6 allele frequency and tamoxifen metabolite concentrations were observed. Patients who had CYP2D6 IM and PM phenotype profile were given recommendation to adjust tamoxifen dose to 40 mg daily, while patients who were clinically ineligible for tamoxifen dose increase according to clinical guidelines were switched to aromatase inhibitor. This example shows the effectiveness of adjusting tamoxifen dosage as the first line of action for patients who are clinically eligible to still consume the drug. Patients who received tamoxifen dose adjustment were monitored to ensure safety from potential side effects associated with tamoxifen.

### Materials and Methods

### Study Participants

Patients were recruited from SJH Initiative, MRCCC Siloam Hospital Jakarta, Indonesia, from October 2019 to April 2021 (n=151). The inclusion criteria of this study were as follows: (1) patient was diagnosed with ER+ breast cancer and (2) had consumed tamoxifen for at least eight weeks. Patients who fulfilled the inclusion criteria were offered to participate in the study and informed consent was obtained. Flow of recruitment steps is shown in FIG. 5. Ethnicities reported in this study were self-reported, participants who identified with two or more ethnicities were categorized as mixed races.

### DNA Extraction

Buccal swab sample was obtained from the patient for CYP2D6 genotyping using ORAcollect-DNA OCR-100 (DNA Genotek) swab. Genomic DNA were extracted from buccal swab samples using Monarch Genomic DNA Purification Kit (NEB #T3010) following the manufacturer's instructions. Concentration of gDNA extracts were quantified using BioDrop spectrophotometer. Acceptance criteria to further process the DNA extract for genotyping, include: (1) total DNA yield 500 ng, (2) A260/280 ratio 1.75, and (3) A260/230 ratio 1.75.

### CYP2D6 Genotyping

CYP2D6 genotyping was performed using Nala PGx Core™, a Lab-Developed Test genotyping panel consisting of four pharmacogenes: CYP2D6, CYP2C19, CYP2C9 and SLCO1B1. CYP2D6 variants that were genotyped in this test included rs35742686, rs59421388, rs3892097, rs5030656, rs72549352, rs5030655, rs28371725, rs16947, rs1065852, rs267608319, rs769258, rs5030865, rs1135840, total copy number of intron 2 and a detection for the presence of exon 9 conversion. Genomic DNA extracts were diluted to 2 ng/uL and added as template for Nala PGx Core™ qPCR runs on Bio-Rad CFX96 Touch™ Real-Time PCR Detection System. CYP2D6 haplotypes, diplotypes and phenotypes were inferred by Nala Clinical Decision Support™ which is a class A medical device (Health Sciences Authority, Singapore) compatible with Nala PGx Core™ qPCR output.

### Measurement of Tamoxifen Metabolites

Finger-prick blood sample was obtained using Volumetric Absorptive Microsampling (VAMS) technique. VAMS extraction was performed in methanol by sonication-assisted extraction method for 25 minutes after 2 hours of VAMS drying. Separation was carried out using Acquity UPLC BEH C1s column (2.1×100 mm; 1.7 μm), with a flow rate of 0.2 mL/minute, and the mobile phase gradient of formic acid 0.1% combined with formic acid 0.1% in acetonitrile for 5 minutes. The UPLC-MS/MS Waters Xevo TQD Triple Quadrupole with MassLynx Software controller (Waters, Milford, USA) was employed in metabolites measurement. Mass detection was carried out utilizing Triple Quadrupole (TOD) with Multiple Reaction Monitoring (MRM) analysis modes and an electrospray ionization source using positive mode. The method was developed in the Bioavailability and Bioequivalence Laboratory of Universitas Indonesia and validated according to FDA and EMA guidelines. The multiple reaction monitoring (MRM) value were set at m/z 372.28>72.22 for TAM; 374.29>58.22 for END; 388.29>72.19 for 4-HT; 358.22>58.09 for NDT; and 260.20>116.20 for propranolol as the internal standard.

### Patient Follow Up

Patients with IM or PM CYP2D6 profile who were clinically ineligible for tamoxifen dose increase were switched to aromatase inhibitor (n=18) and were not followed up further for side effects monitoring and metabolite levels changes. This group of patients were determined based on clinical judgement according to the available guidelines by The National Surgical Oncologist Organization and Ministry of Health in Indonesia (Komite Penanggulangan Kanker Nasional, n.d.), National Comprehensive Cancer Network (NCCN, 2021), and British Columbia Cancer Agency. IM or PM patients who did not have any contraindications to tamoxifen were given a recommendation to adjust its dose to 40 mg/day (n=26), while UMs and NMs remained with the normal 20 mg/day recommended dose (n=81). Tamoxifen metabolites levels in the study participants who were given 40 mg/day of tamoxifen were measured eight weeks post dose adjustment. Endocrine symptoms which were possible side effects of tamoxifen therapy were also monitored in patients who received tamoxifen dose adjustment to 40 mg daily using the FACT-ES questionnaire.

### Data Analysis

Data and statistical analysis were performed using Microsoft® Excel® for Microsoft 365 and R version 4.0.3. Deviation from Hardy-Weinberg equilibrium was performed on the haplotype frequencies using the chi-square statistical test, where Bonferonni correction was applied to determine the p-value threshold for significant deviation. Analysis of Variance (ANOVA) test was used to see if metabolite levels distribution at baseline were statistically different across all metabolites, followed by a paired T-test between each pair of metabolites when significance was found. Distribution of metabolite levels before and after dose adjustment was compared using a T-test, and the same test was used to compare the distribution of metabolite levels in IMs post-dose adjustment against NMs (baseline). Concerning symptoms related to endocrine therapy post-dose adjustment on IMs were compared against NMs. Chi-square test was performed per symptom to check for the difference between the two groups.

### Results

### Demographics of Study Participants

Table 17 shows that out of the 151 participants included in the study, most of the participants were 50 years old and below, making up 78.15% of the total respondents. This proportion was followed by participants between 51-59 years old (17.88%). A small number of older participants with age ≥60 years (3.97%) was also observed. The majority of participants consisted of individuals with Chinese (33.77%) and Javanese (25.17%) descents. Participants with multiethnic and multiracial descents were also observed (16.56%), followed by small numbers of other Indonesian ethnicities such as Sundanese (5.96%), Batak (5.3%), Betawi (3.31%), Minang (3.31%), Ambonese (1.32%), and South Sumatran (1.32%). Among these participants, 47.33% underwent lumpectomy (also known as breast conserving surgery), while 44% underwent mastectomy (total removal of breast tissue). Aside from surgical intervention, 66.67% of these participants underwent adjuvant post-operative radiotherapy and 50% underwent adjuvant chemotherapy. Respondents were mostly still in the early stage of breast cancer during the time of recruitment, with proportion as follows: stage 1 (27.15%), stage IIa (23.84%), and stage IIb (13.91%). Participants who were enrolled to the study and were in the later stage of breast cancer were also observed, with proportion as follows: stage IIIa (7.95%), IIIb (5.96%), and stage IV (7.95%). About half of the study participants (50.33%) were enrolled within 12 months after initial diagnosis of breast cancer. The other participants were enrolled within 13-24 (15.23%), 25-36 (13.25%), and 37-48 (9.27%) months after initial diagnosis, with a proportion of patients who had been diagnosed for longer than four years ago (10.6%). According to the available biopsy data, 44.37% of the participants had moderately differentiated tumors, while 27.81% and 11.92% of the participants had poorly and moderately differentiated tumors, respectively.

TABLE 17
Study respondents demographics

| | n | % |
|---|---|---|
| Age | | |
| <40 | 23 | 15.33% |
| 40-49 | 88 | 58.67% |
| 50-59 | 33 | 22.00% |
| >59 | 6 | 4.00% |
| Menopausal status** | | |
| Premenopausal | 54 | 36.00% |
| Post-menopausal | 96 | 64.00% |
| Menarche | | |
| 7-11 years old | 24 | 16.00% |
| 12-13 years old | 83 | 55.33% |
| >13 years old | 37 | 24.67% |
| NA* | 6 | 4.00% |
| Race | | |
| Ambon | 2 | 1.32% |
| Batak | 8 | 5.30% |
| Betawi | 5 | 3.31% |
| Chinese | 51 | 33.77% |
| Javanese | 38 | 25.17% |
| Minangkabau | 5 | 3.31% |
| Palembang | 2 | 1.32% |
| Sunda | 9 | 5.96% |
| Mixed races | 25 | 16.56% |
| NA* | 6 | 3.97% |
| Past Breast Cancer Treatment | | |
| Lumpectomy | 7 | 4.67% |
| Lumpectomy, chemoterapy | 2 | 1.33% |
| Lumpectomy, radiotherapy | 34 | 22.67% |
| Lumpectomy, chemotherapy, radiotherapy | 23 | 15.33% |
| Mastectomy | 18 | 12.00% |
| Mastectomy, chemoterapy | 16 | 10.67% |
| Mastectomy, radiotherapy | 5 | 3.33% |
| Mastectomy, radiotherapy, chemoterapy | 25 | 16.67% |
| Mastectomy, lumpectomy, radiotherapy, chemotherapy | 2 | 1.33% |
| Radiotherapy | 9 | 6.00% |
| Chemotherapy | 2 | 1.33% |
| Radiotherapy, chemotherapy | 5 | 3.33% |
| NA* | 2 | 1.33% |
| Stage | | |
| ST 0 | 0 | 0% |
| ST I | 34 | 22.67% |
| ST IIA | 48 | 32.00% |
| ST IIB | 17 | 11.33% |
| ST IIIA | 9 | 6.00% |
| ST IIIB | 11 | 7.33% |
| ST IIIC | 2 | 1.33% |
| ST IV | 12 | 8.00% |
| NA* | 17 | 11.33% |
| Time Recruited from Diagnosis (Months) | | |
| 1-12 | 76 | 50.33% |
| 13-24 | 23 | 15.23% |
| 25-36 | 20 | 13.25% |
| 37-48 | 14 | 9.27% |
| >48 | 16 | 10.60% |
| NA* | 1 | 0.66% |
| Tumor Grade | | |
| Well differentiated/Grade 1 | 18 | 11.92% |
| Moderately differentiated/Grade 2 | 67 | 44.37% |
| Poorly differentiated/Grade 3 | 42 | 27.81% |
| NA* | 23 | 15.33% |

*NA: data not available;
**this study includes both pre- and post-menopausal women who were taking tamoxifen by the time of study recruitment

### CYP2D6 Haplotype Distribution

All haplotypes observed were in Hardy-Weinberg equilibrium (p-value >0.005). CYP2D6*10 was found to be the most abundant haplotype in the population (0.288, n=83/288), followed by CYP2D6*36 (0.253, n=73/288). Compared to PharmGKB database of the East Asian population, *10 was lower, but *36 was much higher in this study compared to the frequency reported by the database, 0.012 (FIG. 6). The reference haplotype CYP2D6*1 was observed with frequency of 0.233 (n=67/288), and other haplotypes were also observed with frequencies as follows: *2 (0.128, n=37/288), *41 (0.045, n=13/288), *5 (0.021, n=6/288), *3 (0.014, n=4/288), *39 (0.007, n=2/288), *4A (0.007, n=2/288), and *14 (0.003, n=1/288).

### CYP2D6 Diplotype Distribution

The results here demonstrated *10/*36 (0.236, n=34/144) as the most abundant diplotype in the population, followed by *1/*36 (0.132, n:=19/144) (Table 18). Other diplotypes that were observed in this study with diplotype frequencies between 0.1-0.05 were as follows: *2/*10 (0.097, n=14/144), *1/*1 (0.09, n=13/144), *21*36 (0.083, n=12/144), *1/*10 (0.076, n=11/144), and *10/*10 (0.065, n=9/144). Other diplotypes observed had frequencies lower than 0.05. The list of relevant diplotypes can be found in Table 18.

TABLE 18
CYP2D6 diplotype frequencies observed

| | | Counts | |
|---|---|---|---|
| Diplotype | Phenotype | (N total = 144) | Frequency |
| *10/*36 | Intermediate Metabolizer | 34 | 23.6% |
| *1/*36 | Normal Metabolizer | 19 | 13.2% |
| *2/*10 | Normal Metabolizer | 14 | 9.7% |
| *1/*1 | Normal Metabolizer | 13 | 9.0% |
| *2/*36 | Normal Metabolizer | 12 | 8.3% |
| *1/*10 | Normal Metabolizer | 11 | 7.6% |
| *10/*10 | Normal Metabolizer | 9 | 6.5% |
| Others\hat{ } | | 41 | 22.2% |

\hat{ }Other diplotypes were observed with frequency less than 0.05, these diplotypes were *1/*2, *36/*41, *1/*41, *10/*41, *1/*5, *2/*2, *3/*36, *5/*10, *5/*41, *1/*3, *1/*4A, *14/*36, *2/*3, *2/*39, *2/*41, *36/*39, and *4A/*10

### CYP2D6 Phenotypes Distribution

The present findings show that among the 150 patients genotyped, 40.67% (n=61/150) were IMs. This is much higher than the current known global prevalence of IMs which is between 0.4-11%. The frequency of NMs observed in this study was 54% (n=81/150). PMs were also observed in the population at 1.33% (n=61/150) (FIG. 7). Ultrarapid metabolizers were not observed among the participants in this study. Distribution of the CYP2D6 phenotypes among major ethnicities in the participants showed a higher proportion of IMs in Chinese (56.86%, n=29/51) compared to other ethnicities such as Javanese (23.68%, n=9/38). PM was observed in the Javanese group with 2.63% frequency (n=1). Ethnicities with participant counts less than 10 were grouped as others, due to inefficient number of samples to conclude allele frequencies. Mixed races group showed 37.50% proportion of IM (n=6/16). Among all major ethnicity groups, only Chinese ethnicity group displayed a greater proportion of IM compared to NMV (FIG. 8).

### Tamoxifen Metabolite Concentration

Endoxifen levels among the three metabolizers were significantly different (p-value=0.00307, Table 19). The rest of the metabolites did not show any statistically significant distribution among phenotypes (p-value=0.964, 0.461, 0.443 for tamoxifen, 4-hydroxtamoxifen, and N-desrnethyltamoxifen, respectively). T-test performed on endoxifen levels for each phenotype pair displayed significant difference among all phenotype pairs (p-value=6.26×10⁻⁵, 9.12×10⁻⁵, and 4.714×10⁻³ for NM-PM, NM-IM, and IM-PM, respectively), demonstrating distinction of endoxifen levels across different phenotypes (FIG. 9). After grouping the endoxifen levels into five quintiles, it w as revealed that the highest number of IMs fall into the lowest quintile while the highest number of NMs fall into the highest quintile.

TABLE 19
Summary of metabolite levels in relation to CYP2D6 metabolizer profiles

| CYP2D6 | | Peripheral Whole Blood Concentration (ng/mL) | | | |
|---|---|---|---|---|---|
| Phenotype | | Tamoxifen | Endoxifen | 4OH-tam | ND-tam |
| Normal | SD | 35.21 | 6.62 | 1.46 | 56.83 |
| Metabolizer | Median | 77.46 | 11.98 | 3.07 | 240.59 |
| (N = 81) | Range | 31.22-170.82 | 3.55-34.77 | 1.5-7.66 | 80.63-321.88 |
| Intermediate | SD | 37.20 | 4.35 | 1.67 | 58.01 |
| Metabolizer | Median | 81.72 | 8.33 | 3.27 | 241.55 |
| (N = 61) | Range | 14.22-210.39 | 3.17-22.97 | 1.5-9.31 | 77.61-337.29 |
| Poor | SD | 33.93 | 0.83 | 0.26 | 90.44 |
| Metabolizer | Median | 91.49 | 4.52 | 3.24 | 276.45 |
| (N = 2) | Range | 67.49-115.48 | 3.94-5.11 | 3.06-3.43 | 212.5-340.41 |

| | | | | |
|---|---|---|---|---|
| p-value (ANOVA) | 0.964 | 0.00307* | 0.461 | 0.443 |

*Statistically significant p-value was observed among phenotype groups for endoxifen level difference

### Follow Up Action Following PGx Testing

Among 66 IM or PM participants who were given the recommendation to modify their medication based on their CYP2D6 phenotype (FIG. 10), 18 patients (27.3%, n=18/66) had their medication switched to aromatase inhibitors based on clinical guidelines or certain medical procedure such as post Ovarian Function Suppression (OFS) endocrine therapy. 38 patients (57.6%, n=38/66) were recommended by their physicians to adjust their tamoxifen dosage from 20 mg daily to 40 mg daily, while the remaining participants who did not follow the genotype-guided recommendation either passed away or experienced recurrence, thus they had to dismiss their adjuvant therapy temporarily (15.2%, n=10/66).

### Metabolite Levels Post Dose Adjustment

26 patients who took 40 mg of tamoxifen daily for two months all experienced an increase in metabolite levels. After dose adjustment, the range of tamoxifen metabolites increased as follows: tamoxifen levels from 14.22-210.39 ng/mL to 80.59-254.96 ng/mL; endoxifen levels from 3.17-22.97 ng/mL to 7.68-23.36 ng/mL; 4-hydroxytamoxifen levels from 1.5-9.31 ng/mL to 3.34-12.99 ng/mL, and N-desmethyltamoxifen levels from 77.61-337.29 ng/mL to 236.8-501.9 ng/mL (FIG. 11). Metabolite levels before and after dose adjustment had p-value <0.05, demonstrating statistically significant differences before and after dose adjustment across all metabolites.

The metabolite levels in IMs (n=26) post dose adjustment were compared against NMs (n=81) as the baseline, showing indeed a significant difference between the two groups (p-value <0.05) for all metabolites except endoxifen (p-value=0.4135). The distribution of endoxifen levels in IMs post dose adjustment (7.68-23.36 ng/mL) were similar to the endoxifen levels in NMs (3.55-34.77 ng/mL) at baseline (FIG. 12).

### Side Effects Post Dose Adjustment

The most commonly reported treatment side effects in IMs were weight gain and mood swings, which are related to endocrine therapy. These occurred in 65.83% of participants who received 40 mg of tamoxifen daily (n=17/26). Other common symptoms related to hormonal changes were also observed in participants who received 40 mg of tamoxifen daily such as hotflush (50%, n=13/26), cold sweats (19.23%, n=5/26), night sweats (26.92%, n=7/26), vaginal discharge (42.31%, n=11/26), vaginal itching or irritation (15.38%, n=4/26), vaginal bleeding or spotting (23.08%, n=6/26), vaginal dryness (11.54%, n=3/26), pain or discomfort during intercourse (3.85%, n=1/26), lost interest in sex (15.38%, n=4/26), breast sensitivity or tenderness (53.85%, n=14/26), and irritability (61.54%, n=16/26). Other symptoms that might be related to endocrine therapy were also observed, such as lightheaded/dizziness (34.62%, n=9/26), vomiting (3.85%, n=1/26), headaches (53.85%, n=14/26), bloating (46.15%, n=12/26), and pain in joints (50%, n=13/26). No post-dose adjustment participants reported diarrhea.

The most commonly reported side effect in the patient group that took 20 mg of tamoxifen daily was mood swings, occurring in 74.19% of the respondents (n=23/31), although they did not receive any treatment adjustments. Other common symptoms related to hormonal changes were also observed in NM participants such has hotflush (35.48%, n=11/31), cold sweats (12.9%, n=4/31), night sweats (29.03%, n=9/31), vaginal discharge (38.71%, n=12/31), vaginal itching or irritation (22.58%, n=7/31), vaginal bleeding or spotting (16.13%, n=5/31), vaginal dryness (32.26%, n=10/31), pain or discomfort during intercourse (51.61%, n=16/31), lost interest in sex (64.52%, n=20/31), breast sensitivity or tenderness (41.94%, n=13/31), and irritability (58.06%, n=18/31). Other symptoms that might be related to endocrine therapy were also observed, such as lightheaded/dizziness (35.48%, n==11/31), vomiting (6.45%, n=2/31), diarrhea (3.23%, n=1/31), headaches (29.03%, n=9/31), bloating (38.71%, n=12/31), and pain in joints 67.74%, n=21/31).

T-test performed between symptoms experienced by participants receiving dose adjustment to 40 mg daily and participants taking 20 mg daily resulted in two symptoms (pain or discomfort during intercourse and lost interest in sex) with statistical significance between the two groups. Other than these two symptoms, the other symptoms did not have significant difference among the two groups, indicating that dose escalation up to 40 mg daily did not increase potential toxicity or side effects (Table 20). Thrombophlebitis, thrombosis, endometriosis, and endometrial cancer were also some of the most concerning side effects of tamoxifen, and none of these side effects were observed in the observed population.

TABLE 20
Number and percentage of patient responses related to adverse
events in FACT-ES post eight weeks after dose adjustment.

| | NM participants who | | IM participants who | | |
|---|---|---|---|---|---|
| | received 20 mg of | | received 40 mg of | | |
| | tamoxifen daily (N = 31) | | tamoxifen daily (N = 22) | | |
| | Patients | Patients | Patients | Patients | |
| | reported | reported | reported | reported | |
| | side effect | side effect | side effect | side effect | |
| Symptoms | (n) | (%) | (n) | (%) | p-value |
| Hot Flashes | 11 | 35.48% | 13 | 50.00% | 0.269361 |
| Cold Sweats | 4 | 12.90% | 5 | 19.23% | 0.717648 |
| Night sweats | 9 | 29.03% | 7 | 26.92% | 0.86249 |
| Vaginal discharge | 12 | 38.71% | 11 | 42.31% | 0.777297 |
| Vaginal itching/irritation | 7 | 22.58% | 4 | 15.38% | 0.492987 |
| Vaginal bleeding or spotting | 5 | 16.13% | 6 | 23.08% | 0.507122 |
| Vaginal dryness | 10 | 32.26% | 3 | 11.54% | 0.063252 |
| Pain or discomfort with | 16 | 51.61% | 1 | 3.85% | 8.48 × 10⁻⁵* |
| intercourse* | | | | | |
| Lost interest in sex | 20 | 64.52% | 4 | 15.38% | 0.005461* |
| Weight gain | 20 | 64.52% | 17 | 65.38% | 1 |
| Lightheaded (dizzy) | 11 | 35.48% | 9 | 34.62% | 1 |
| Vomiting | 2 | 6.45% | 1 | 3.85% | 1 |
| Diarrhea | 1 | 3.23% | 0 | 0.00% | 1 |
| Headaches | 9 | 29.03% | 14 | 53.85% | 0.057089 |
| Bloating | 12 | 38.71% | 12 | 46.15% | 0.571608 |
| Breast sensitivity/tenderness | 13 | 41.94% | 14 | 53.85% | 0.371093 |
| Mood swings | 23 | 74.19% | 17 | 65.38% | 0.470842 |
| Irritable | 18 | 58.06% | 16 | 61.54% | 0.791337 |
| Pain in joints | 21 | 67.74% | 13 | 50.00% | 0.173783 |

*Statistically significant p-values were observed between IMs who have received tamoxifen dose adjustment and NMs who took the standard dose
*Statistically significant p-value was observed.

### Discussion

This example observes the distribution of CYP2D6 genotypes and phenotypes across Indonesian women diagnosed with ER+ breast cancer who were taking tamoxifen as adjuvant therapy. Our respondents were mostly of Chinese and Javanese descent. Chinese ethnicity group in this example's population showed a higher proportion of intermediate metabolizers, while the Javanese ethnicity group was dominated by normal metabolizers (FIG. 8). The proportion of Ms in Indonesian Chinese included in this example was higher than a similar study conducted on Han Chinese population, which was 45.38%. Ethnicity differences may play a role in contributing to the differences between the findings in this study and other similar studies conducted in different populations. Caucasians may have a higher proportion of normal metabolizers compared to other races/ethnicities though the frequencies are slightly varied depending on the geographical location where the studies were conducted.

The results reported CYP2D6*10 as the most common CYP2D6 haplotype. Some studies have suggested that this allele increases the risk of breast cancer recurrence for those taking tamoxifen as adjuvant therapy. A study conducted in the Han Chinese population showed that the frequency of CYP2D6*10 in this population was 45.7%, higher than the frequency of CYP2D6*10 observed in this study (28.8%). Another important highlight was the relatively high frequency of *36 allele observed in this study (0.253) compared to the observed frequency in the PharmGKB database (0.012). Compared to other Asian population, a study conducted in Hong Kong population also recorded a relatively high frequency of CYP2D6*36 which is 34.1%. Although some *36 allele contributed to normal metabolizer status profile, our study observed *10/*36 diplotype as the diplotype with highest frequency (0.236), and this diplotype translates as IM phenotype which suggested that *36 may play an important role in constructing IM phenotype profiles in Indonesian population. These findings suggested that Indonesian population might be at higher risk of experiencing ineffectiveness of tamoxifen therapy. This was also supported by the high proportion of CYP2D6 IMs (40.67%) compared to other studies conducted in different populations. This was also much higher than the current known global prevalence of IMs which is between 0.4-11%. Even so, some populations also reported a higher proportion of IMs, suggesting that different populations composed of various ethnicities may play a role in genetic make-up differences of CYP2D6. Compared to our result, a similar study conducted in Thailand population showed a relatively high frequency compared to the global prevalence (29.1%), implying that East Asian population may have relatively higher frequency of IM. The frequency of NMs observed in this study (54%) was also lower than the current known global prevalence which is between 67-90%.

Different metabolites of tamoxifen and their levels were a predictor of tamoxifen's efficacy, especially endoxifen levels. Lower endoxifen levels in IMs may indicate lower efficacy of tamoxifen in preventing recurrence. Compared to a previous study, the average value of endoxifen levels in IMs observed in this study was higher. The previous study observed the average endoxifen level of IMs to be 8.1 ng/mL while this study recorded an average at 9.6 ng/mL. However, a study conducted in Swedish population found a range of endoxifen level between 2.3-16 ng/mL, while another study conducted in Singaporean population displayed a range between 1.74-42.8 ng/mL. These suggested that studies conducted with similar interventions but in different populations may find different ranges of metabolite levels.

It was recommended here that IMs and PMs adjust their tamoxifen dosage or switch prescription to aromatase inhibitors for patients that were clinically ineligible for consumption of tamoxifen. Patients who received tamoxifen dose adjustment to 40 mg daily were specifically monitored, and results have shown that participants who received 40 mg of tamoxifen daily all experienced a significant increase across all metabolite levels, including endoxifen levels. This suggested that increasing tamoxifen intake can elevate endoxifen levels as expected and may play a role in increasing the therapeutic effect of tamoxifen. The distribution of endoxifen level in IMs post dose adjustment were similar to the endoxifen level in NMs at the baseline, suggesting that increasing tamoxifen dosage to 40 mg daily for IM participants had successfully let IM participants reach the expected endoxifen levels as observed in NMs.

Gynecological side effects similar to menopausal symptoms such as hot flushes, vaginal dryness, and endometriosis were commonly observed in patients taking tamoxifen. According to the survey for endocrine symptoms in this study, most participants experienced mild to moderate degree of endocrine symptoms. Despite some of the IM respondents who received dose increase reporting experiencing hot flush, no respondents reported dismissing tamoxifen intake due to the symptom. Hot flush was also commonly reported in patients taking the standard dose of tamoxifen therapy, which means increasing tamoxifen dose does not change side effects of the drug distinctly. Thrombophlebitis, thrombosis, endometriosis, and endometrial cancer were also some of the most concerning side effects of tamoxifen, since they fatally affect patients' quality of life and life expectancy. None of these side effects were observed in the observed population, but this might also be underestimated due to the short period of follow up on this study. Other studies who have tried to observe tamoxifen side effects occurring in patients with dose increase also concluded that increasing tamoxifen dose did not result in toxicity or short-term increase in side effects.

These findings concluded that tamoxifen dose adjustment is beneficial enough to increase potential therapeutic effect through the increase of metabolite levels, with no fatal side effects recorded. Although CPIC guideline recommended the first course of action to switch to aromatase inhibitors, our finding demonstrated that tamoxifen dose adjustment is adequate.

This is favourable due to: 1) the higher likelihood of potential side effects from aromatase inhibitors than tamoxifen, 2) lower price of tamoxifen than aromatase inhibitors to allow cost-effectiveness in periodical prescriptions throughout the period of adjuvant therapy.

### EXAMPLE 4

Further examples of the various components of the Nala PGx Core™ Kit are provided in Tables 21-40.

TABLE 21
SNP1
(rs1065852)

| | | | | | Conc | | | Amount |
|---|---|---|---|---|---|---|---|---|
| | | | | | after | | | (nmole) |
| | | | | Measured | 10× | Per | Final | per rx |
| | | | | conc. | dilution | Reaction | conc | (25 ul) |
| Component | Name | Direction 5′-3′ | Specifications | (uM) | (UM) | (uL) | (uM) | |
| Master mix | SSO | NA | #1725285 | NA | NA | 12.5 | 1× | NA |
| | Advanced | | | | | | | |
| | Universal | | | | | | | |
| | Probes | | | | | | | |
| | Supermix | | | | | | | |
| Primer F | rs1065852_ | 5′ GACCTGATGCACCG | 17 bp | 97.25 | 9.725 | 0.5 | 0.195 | 0.0048 |
| | F1 | GCG 3′ | (Tm = 59.8) | | | | | 6 |
| | (SNP1_F1) | | | | | | | |
| Primer R | rs1065852_ | 5′ ATG TAT AAA | 19 bp | 109.48 | 10.948 | 0.5 | 0.219 | 0.0054 |
| | R5 | TGC CCT TCT C 3′ | (Tm = 50.9) | | | | | 7 |
| | (SNP1_R5) | | | | | | | |
| Probe A | rs1065852_ | 5′ | [6FAM]-17 bp- | 92.85 | 9.285 | 1 | 0.371 | 0.0092 |
| | P4_WT_R | 6-FAM/CTGGTGGGTA | IBFQ] | | | | | 8 |
| | SNP1_P4_ | GCGTGCA/BHQ13′ | (Tm = 57.3) | | | | | |
| | WT_R) | | | | | | | |
| Probe B | rs1065852_ | 5′ HEX- | [HEX]-19 bp- | 94.85 | 9.485 | 0.75 | 0.285 | 0.0071 |
| | P1_M_R_HEX | CCTGGTGAGTAGCG | [IBFQ] | | | | | 1 |
| | (SNP1_P1_ | TGCAG-IBFQ 3′ | (Tm = 61.6) | | | | | |
| | M_R_HEX) | | | | | | | |
| Tris-EDTA | EDTA, pH | NA | 1^{st} Base | | | 7.75 | NA | NA |
| buffer | 1× Tris- | | | | | | | |
| | EDTA (TE) | | | | | | | |
| | Buffer with | | | | | | | |
| | reduced | | | | | | | |
| | 8.0, | | | | | | | |
| | Biotechnology | | | | | | | |
| | Grade, | | | | | | | |
| | 1L (#CUS- | | | | | | | |
| | 3022-1 × 1L) | | | | | | | |
| Template_ | T1_WT_ | ACCGGCGCCAACGC | gblock-335 bp | | | | 100000 | \|100000 |
| WT | Extended for | GAGTGTCCTGCCTG | | | | | | |
| | R5 | GTCCTCTGTGCCTG | | | | | | |
| | | GTGGGGTGGGGGT | | | | | | |
| | | GCCAGGTGTGTCCA | | | | | | |
| | | GAGGAGCCCATTTG | | | | | | |
| | | GTAGTGAGGCAGGT | | | | | | |
| | | ATGGGGCTAGAAGC | | | | | | |
| | | ACTGGTGCCCCTGG | | | | | | |
| | | CCGTGATAGTGGCC | | | | | | |
| | | ATCTTCCTGCTCCT | | | | | | |
| | | GGTGGACCTGATGC | | | | | | |
| | | TGGGCTGCACGCTA | | | | | | |
| | | CCCACCAGGCCCCC | | | | | | |
| | | TGCCACTGCCCGGG | | | | | | |
| | | CTGGGCAACCTGCT | | | | | | |
| | | GCATGTGGACTTCC | | | | | | |
| | | AGAACACACCATAC | | | | | | |
| | | TGCTTCGACCAGGT | | | | | | |
| | | GAGGGAGGAGGTC | | | | | | |
| | | CTGGAGGGCGGCA | | | | | | |
| | | GAGGTGCTGAGGCT | | | | | | |
| | | CCCCTACCAGAAGC | | | | | | |
| | | AAACATGGATGGTG | | | | | | |
| | | GG | | | | | | |
| Template_ | T1_MT_Ext | GAGTGTCCTGCCTG | gblock-33 5bp | | | | 100000 | 100000 |
| M | ended for | GTCCTCTGTGCCTG | | | | | | |
| | R5 | GTGGGGTGGGGGT | | | | | | |

| | | |
|---|---|---|
| | | GCCAGGTGTGTCCA |
| | | GAGGAGCCCATTTG |
| | | GTAGTGAGGCAGGT |
| | | ATGGGGCTAGAAGC |
| | | ACTGGTGCCCCTGG |
| | | CCATGATAGTGGCC |
| | | ATCTTCCTGCTCCT |
| | | GGTGGACCTGATGC |
| | | ACCGGCGCCAACGC |
| | | TGGGCTGCACGCTA |
| | | CTCACCAGGCCCCC |
| | | TGCCACTGCCCGGG |
| | | CTGGGCAACCTGCT |
| | | GCATGTGGACTTCC |
| | | AGAACACACCATAC |
| | | TGCTTCGACCAGGT |
| | | GAGGGAGGAGGTC |
| | | CTGGAGGGCGGCA |
| | | GAGGTGCTGAGGCT |
| | | CCCCTACCAGAAGC |
| | | AAACATGGATGGTG |
| | | GG |
| HapMap_ | NA12762, | |
| Homo WT | NA21114 | |
| HapMap_ | NA19143, | |
| Hetero | NA18961 | |
| HapMap_ | NA18550, | |
| Homo M | NA11992 | |

TABLE 22
SNP2
(rs5030655)

| | | | | | Conc | | | |
|---|---|---|---|---|---|---|---|---|
| | | | | | after | | | Amount |
| | | | | Measured | 10× | Per | Final | (nmole) |
| | | | | conc. | dilution | Reaction | conc. | per rx |
| Component | Name | Direction 5′-3′ | Specifications | (uM) | (uM) | (uL) | (uM) | (25 ul) |
| Master mix | SSO | NA | #1725285 | | | 12.5 | 1× | NA |
| | Advanced | | | | | | | |
| | Universal | | | | | | | |
| | Probes | | | | | | | |
| | Supermix | | | | | | | |
| Primer F | rs5030655_ | 5′ | 18bp | 96.90 | 9.690 | 0.5 | 0.194 | 0.0048 |
| | F5 | TTGCGCAACTTGGG | (Tm = 58.4) | | | | | 5 |
| | (SNP2_F5) | CCTG 3′ | | | | | | |
| Primer R | rs5030655_ | 5′ | 17bp | 88.95 | 8.895 | 1 | 0.356 | 0.0088 |
| | R2 | ACCCACCGGAGTGG | (Tm = 57.3) | | | | | 9 |
| | (SNP2_R2) | TTG 3′ | | | | | | |
| Probe A | rs5030655_ | CTGCTCCAG/BHQ13′ | [6FAM]-20 bp- | 106.39 | 10.639 | 2.5 | 1.064 | 0.0266 |
| | P3_WT_R | 5′6- FAM/TCGGTCACCCA | [BHQ1] | | | | | 0 |
| | (SNP2_P3_ | | (Tm = 64.6) | | | | | |
| | WT_R) | | | | | | | |
| Probe B | rs5030655_ | 5′ HEX- | [HEX]-19 bp- | 104.49 | 10.449 | 1.5 | 0.627 | 0.0156 |
| | P3_M_R_ | TCGGTCACCCCTGC | [IBFQ] | | | | | 7 |
| | HEX | TCCAG-IBFQ 3′ | (Tm = 63.6) | | | | | |
| | (SNP2_P3_ | | | | | | | |
| | M_R_HEX) | | | | | | | |
| Tris-EDTA | 1× Tris- | NA | 1^{st} Base | | | 5.00 | NA | NA |
| buffer | EDTA | | | | | | | |
| | (TE) | | | | | | | |
| | Buffer | | | | | | | |
| | with | | | | | | | |
| | reduced | | | | | | | |
| | EDTA, pH | | | | | | | |
| | 8.0, | | | | | | | |
| | Biotechnology | | | | | | | |
| | Grade, 1L | | | | | | | |
| | K#CUS- | | | | | | | |
| | 3022- | | | | | | | |
| | 1 × 1L) | | | | | | | |
| Template_ | CYP2D6 | GAGCCAGGGACTGC | gblock-500 bp | | | | 100000 | 100000 |
| WT | WT T2 | GGGAGACCAGGGG | | | | | | |
| | | GAGCATAGGGTTGG | | | | | | |
| | | AGTGGGTGGTGGAT | | | | | | |
| | | GGTGGGGCTAATGC | | | | | | |
| | | CTTCATGGCCACGC | | | | | | |
| | | GCACGTGCCCGTCC | | | | | | |
| | | CACCCCCAGGGGTG | | | | | | |
| | | TTCCTGGCGCGCTA | | | | | | |
| | | TGGGCCCGCGTGG | | | | | | |
| | | CGCGAGCAGAGGC | | | | | | |
| | | GCTTCTCCGTGTCC | | | | | | |
| | | ACCTTGCGCAACTT | | | | | | |
| | | GGGCCTGGGCAAG | | | | | | |
| | | AAGTCGCTGGAGCA | | | | | | |
| | | GTGGGTGACCGAG | | | | | | |
| | | GAGGCCGCCTGCCT | | | | | | |
| | | TTGTGCCGCCTTCG | | | | | | |
| | | CCAACCACTCCGGT | | | | | | |
| | | GGGTGATGGGCAGA | | | | | | |
| | | AGGGCACAAAGCGG | | | | | | |
| | | GAACTGGGAAGGCG | | | | | | |
| | | GGGGACGGGGAAG | | | | | | |
| | | GCGACCCCTTACCC | | | | | | |
| | | GCATCTCCCACCCC | | | | | | |
| | | CAGGACGCCCCTTT | | | | | | |
| | | CGCCCCAACGGTCT | | | | | | |
| | | CTTGGACAAAGCCG | | | | | | |
| | | TGAGCAACGTGATC | | | | | | |
| | | GCCTCCCTCACCTG | | | | | | |
| | | CGGGCGCCGCTTC | | | | | | |
| | | GAGTACGACGACCC | | | | | | |
| | | TCGCTTCCTCAGGC | | | | | | |
| | | TGCTGGACCTAGCT | | | | | | |
| | | CAGGAGGGACTGAA | | | | | | |
| | | GGAGGAGTCGGGC | | | | | | |
| | | TTT | | | | | | |
| Template_M | CYP2D6_ | GAGCCAGGGACTGC | gblock-500 bp | | | | 100000 | 100000 |
| | M T2 | GGGAGACCAGGGG | | | | | | |
| | | GAGCATAGGGTTGG | | | | | | |
| | | AGTGGGTGGTGGAT | | | | | | |
| | | GGTGGGGCTAATGC | | | | | | |
| | | CTTCATGGCCACGC | | | | | | |
| | | GCACGTGCCCGTCC | | | | | | |
| | | CACCCCCAGGGGTG | | | | | | |
| | | TTCCTGGCGCGCTA | | | | | | |
| | | TGGGCCCGCGTGG | | | | | | |
| | | CGCGAGCAGAGGC | | | | | | |
| | | GCTTCTCCGTGTCC | | | | | | |
| | | ACCTTGCGCAACTT | | | | | | |
| | | GGGCCTGGGCAAG | | | | | | |
| | | AAGTCGCTGGAGCA | | | | | | |
| | | GGGGTGACCGAGG | | | | | | |
| | | AGGCCGCCTGCCTT | | | | | | |
| | | TGTGCCGCCTTCGC | | | | | | |
| | | CAACCACTCCAGTG | | | | | | |
| | | GGTGATGGGCAGAA | | | | | | |

| | | |
|---|---|---|
| | | GGGCACAAAGCGG |
| | | GAACTGGGAAGGCG |
| | | GGGGACGGGGAAG |
| | | GCGACCCCTTACCC |
| | | GCATCTCCCACCCC |
| | | CAAGACGCCCCTTT |
| | | CGCCCCAACGGTCT |
| | | CTTGGACAAAGCCG |
| | | TGAGCAACGTGATC |
| | | GCCTCCCTCACCTG |
| | | CGGGCGCCGCTTC |
| | | GAGTACGACGACCC |
| | | TCGCTTCCTCAGGC |
| | | TGCTGGACCTAGCT |
| | | CAGGAGGGACTGAA |
| | | GGAGGAGTCGGGC |
| | | TTT |
| HapMap_ | NA12762, | |
| Homo WT | NA21114 | |
| HapMap_ | NA07357 | |
| Hetero | | |
| HapMap_ | N/A | |
| Homo M | | |

TABLE 23
SNP3
(rs3892097)

| | | | | | Conc | | | |
|---|---|---|---|---|---|---|---|---|
| | | | | | after | | | Amount |
| | | | | Measured | 10× | Per | Final | (nmole) |
| | | | | conc | dilution | Reaction | conc. | per rx |
| Component | Name | Direction 5′-3′ | Specifications | (uM) | (uM) | (uL) | (uM) | (25 ul) |
| Master | SSO | NA | #1725285 | | | 12.5 | 1× | NA |
| mix | Advanced | | | | | | | |
| | Universal | | | | | | | |
| | Probes | | | | | | | |
| | Supermix | | | | | | | |
| Primer F | rs3892097_ | 5′ | 18 bp | 101.23 | 10.123 | 1 | 0.405 | 0.01012 |
| | F2d | GCCGCCTTCGCCAA | (Tm = 62.9) | | | | | |
| | (SNP3_F2d) | CCAC 3′ | | | | | | |
| Primer R | rs3892097_ | 5′ | 19 bp | 96.72 | 9.672 | 1.5 | 0.580 | 0.01451 |
| | R1b | ACGGCTTTGTCCAA | (Tm = 57.5) | | | | | |
| | (SNP3_R1b) | GAGAC 3′ | | | | | | |
| Probe A | rs3892097_ | 5′ | [6FAM]-19 bp- | 106.26 | 10.626 | 2 | 0.850 | 0.02125 |
| | P4_WT_F | 6-FAM/ACCCCCAGGA | [BHQ1] | | | | | |
| | (SNP3_P4_ | CGCCCCTT/BHQ13′ | (Tm = 62.9) | | | | | |
| | WT_F) | | | | | | | |
| Probe B | rs3892097_ | 5′ | [HEX]-19 bp- | 105.52 | 10.552 | 2 | 0.844 | 0.02110 |
| | P1_M_ | HEX/ACCCCCAAGAC | [IBFQ] | | | | | |
| | F_HEX | GCCCCTTT/IBFQ 3′ | (Tm = 61.6) | | | | | |
| | (SNP3_P1_ | | | | | | | |
| | M_F_HEX) | | | | | | | |
| Tris-EDTA | 1× Tris- | | 1^{st} Base | | | 4.00 | NA | NA |
| buffer | EDTA | | | | | | | |
| | (TE) | | | | | | | |
| | Buffer | | | | | | | |
| | with | | | | | | | |
| | reduced | | | | | | | |
| | EDTA, pH | | | | | | | |
| | 8.0, | | | | | | | |
| | Bio- | | | | | | | |
| | technology | | | | | | | |
| | Grade, 1L | | | | | | | |
| | #CUS- | | | | | | | |
| | 3022- | | | | | | | |
| | (1 × 1L) | | | | | | | |
| Template_ | CYP2D6_ | GAGCCAGGGACTGC | gblock-500 bp | | | | 100000 | 100000 |
| WT | WT_T2 | GGGAGACCAGGGG | | | | | | |
| | | GAGCATAGGGTTGG | | | | | | |
| | | AGTGGGTGGTGGAT | | | | | | |
| | | GGTGGGGCTAATGC | | | | | | |
| | | CTTCATGGCCACGC | | | | | | |
| | | GCACGTGCCCGTCC | | | | | | |
| | | CACCCCCAGGGGTG | | | | | | |
| | | TTCCTGGCGCGCTA | | | | | | |
| | | TGGGCCCGCGTGG | | | | | | |
| | | CGCGAGCAGAGGC | | | | | | |
| | | GCTTCTCCGTGTCC | | | | | | |
| | | ACCTTGCGCAACTT | | | | | | |
| | | GGGCCTGGGCAAG | | | | | | |
| | | AAGTCGCTGGAGCA | | | | | | |
| | | GTGGGTGACCGAG | | | | | | |
| | | GAGGCCGCCTGCCT | | | | | | |
| | | TTGTGCCGCCTTCG | | | | | | |
| | | CCAACCACTCCGGT | | | | | | |
| | | GGGTGATGGGCAGA | | | | | | |
| | | AGGGCACAAAGCGG | | | | | | |
| | | GAACTGGGAAGGCG | | | | | | |
| | | GGGGACGGGGAAG | | | | | | |
| | | GCGACCCCTTACCC | | | | | | |
| | | GCATCTCCCACCCC | | | | | | |
| | | CAGGACGCCCCTTT | | | | | | |
| | | CGCCCCAACGGTCT | | | | | | |
| | | CTTGGACAAAGCCG | | | | | | |
| | | TGAGCAACGTGATC | | | | | | |
| | | GCCTCCCTCACCTG | | | | | | |
| | | CGGGCGCCGCTTC | | | | | | |
| | | GAGTACGACGACCC | | | | | | |
| | | TCGCTTCCTCAGGC | | | | | | |
| | | TGCTGGACCTAGCT | | | | | | |
| | | CAGGAGGGACTGAA | | | | | | |
| | | GGAGGAGTCGGGC | | | | | | |
| | | TTT | | | | | | |
| Template_ | CYP2D6_ | GAGCCAGGGACTGC | gblock-500 bp | | | | 100000 | 100000 |
| M | M_T2 | GGGAGACCAGGGG | | | | | | |
| | | GAGCATAGGGTTGG | | | | | | |
| | | AGTGGGTGGTGGAT | | | | | | |
| | | GGTGGGGCTAATGC | | | | | | |
| | | CTTCATGGCCACGC | | | | | | |
| | | GCACGTGCCCGTCC | | | | | | |
| | | CACCCCCAGGGGTG | | | | | | |
| | | TTCCTGGCGCGCTA | | | | | | |
| | | TGGGCCCGCGTGG | | | | | | |
| | | CGCGAGCAGAGGC | | | | | | |
| | | GCTTCTCCGTGTCC | | | | | | |
| | | ACCTTGCGCAACTT | | | | | | |
| | | GGGCCTGGGCAAG | | | | | | |
| | | AAGTCGCTGGAGCA | | | | | | |
| | | GGGGTGACCGAGG | | | | | | |
| | | AGGCCGCCTGCCTT | | | | | | |
| | | TGTGCCGCCTTCGC | | | | | | |
| | | CAACCACTCCAGTG | | | | | | |
| | | GGTGATGGGCAGAA | | | | | | |
| | | GGGCACAAAGCGG | | | | | | |
| | | GAACTGGGAAGGCG | | | | | | |
| | | GGGGACGGGGAAG | | | | | | |
| | | GCGACCCCTTACCC | | | | | | |
| | | GCATCTCCCACCCC | | | | | | |
| | | CAAGACGCCCCTTT | | | | | | |
| | | CGCCCCAACGGTCT | | | | | | |
| | | CTTGGACAAAGCCG | | | | | | |
| | | TGAGCAACGTGATC | | | | | | |
| | | GCCTCCCTCACCTG | | | | | | |
| | | CGGGCGCCGCTTC | | | | | | |
| | | GAGTACGACGACCC | | | | | | |
| | | TCGCTTCCTCAGGC | | | | | | |
| | | TGCTGGACCTAGCT | | | | | | |
| | | CAGGAGGGACTGAA | | | | | | |
| | | GGAGGAGTCGGGC | | | | | | |
| | | TTT | | | | | | |
| HapMap_ | NA21114, | | | | | | | |
| Homo WT | NA19143 | | | | | | | |
| HapMap_ | NA12006, | | | | | | | |
| Hetero | NA12003 | | | | | | | |
| HapMap_ | NA11992 | | | | | | | |
| Homo M | | | | | | | | |

TABLE 24
SNP4
(rs35742686)

| | | | | | Conc | | | |
|---|---|---|---|---|---|---|---|---|
| | | | | | after | | | Amount |
| | | | | Measured | 10× | Per | Final | (nmole) |
| | | | | conc | dilution | Reaction | conc | per rx |
| Component | Name | Direction 5′-3′ | Specifications | (uM) | (uM) | (uL) | (uM) | (25 ul) |
| Master mix | SSO | NA | | | | 12.5 | 1× | NA |
| | Advanced | | | | | | | |
| | Universal | | | | | | | |
| | Probes | | | | | | | |
| | Supermix | | | | | | | |
| Primer F | rs35742686_ | 5′ | 18 bp | 96.99 | 9.699 | 1 | 0.388 | 0.00970 |
| | F1m | GTCCTCGTCCTCCT | (Tm = 58.4) | | | | | |
| | (SNP4_F1m) | GCAT 3′ | | | | | | |
| Primer R | rs35742686_ | 5′ | 18 bp | 88.54 | 8.854 | 0.5 | 0.177 | 0.00443 |
| | R1 | TCAGTCAGGTCTCG | (Tm = 60.8) | | | | | |
| | (SNP4_R1) | GGGG 3′ | | | | | | |
| Probe A | rs357426 | 5′ | [6FAM]-21 bp- | 92.69 | 9.269 | 1 | 0.371 | 0.00927 |
| | 86 P2_WT_R | 6-FAM/TCCCAGGTCAT | [BHQ1] | | | | | |
| | (SNP4_ | CCTGTGCTCA/BHQ1 | (Tm = 63.2) | | | | | |
| | P2_WT_R) | 3′ | | | | | | |
| Probe B | rs35742686_ | 5′ HEX- | [HEX]-18 bp- | 102.11 | 10.211 | 2.25 | 0.919 | 0.02298 |
| | P4_M_ | CAGGTCATCCGTGC | [IBFQ] | | | | | |
| | R_HEX | TCAG-IBFQ 3′ | (Tm = 58.4) | | | | | |
| | (SNP4_P4_ | | | | | | | |
| | M_R_HEX) | | | | | | | |
| Tris-EDTA | 1× Tris- | NA | 1^{st} Base | | | 5.75 | NA | NA |
| buffer | EDTA | | | | | | | |
| | (TE) | | | | | | | |
| | Buffer | | | | | | | |
| | with | | | | | | | |
| | reduced | | | | | | | |
| | EDTA, pH | | | | | | | |
| | 8.0, | | | | | | | |
| | Bio- | | | | | | | |
| | technology | | | | | | | |
| | Grade, 1L | | | | | | | |
| | (#CUS- | | | | | | | |
| | 3022- | | | | | | | |
| | 1 × 1L) | | | | | | | |
| Template_ | CYP2D6_ | CCTGGGTCTACCTG | gblock-500 bp | | | | 100000 | 100000 |
| WT | WT_T3 | GAGATGGCTGGGG | | | | | | |
| | | CCTGAGACTTGTCC | | | | | | |
| | | AGGTGAACGCAGAG | | | | | | |
| | | CACAGGAGGGATTG | | | | | | |
| | | AGACCCCGTTCTGT | | | | | | |
| | | CTGGTGTAGGTGCT | | | | | | |
| | | GAATGCTGTCCCCG | | | | | | |
| | | TCCTCCTGCATATC | | | | | | |
| | | CCAGCGCTGGCTGG | | | | | | |
| | | CAAGGTCCTACGCT | | | | | | |
| | | TCCAAAAGGCTTTC | | | | | | |
| | | CTGACCCAGCTGGA | | | | | | |
| | | TGAGCTGCTAACTG | | | | | | |
| | | AGCACAGGATGACC | | | | | | |
| | | TGGGACCCAGCCCA | | | | | | |
| | | GCCCCCCCGAGACC | | | | | | |
| | | TGACTGAGGCCTTC | | | | | | |
| | | CTGGCAGAGATGGA | | | | | | |
| | | GAAGGTGAGAGTGG | | | | | | |
| | | CTGCCACGGTGGG | | | | | | |
| | | GGGCAAGGGTGGT | | | | | | |
| | | GGGTTGAGCGTCCC | | | | | | |
| | | AGGAGGAATGAGGG | | | | | | |
| | | GAGGCTGGGCAAAA | | | | | | |
| | | GGTTGGACCAGTGC | | | | | | |
| | | ATCACCCGGCGAGC | | | | | | |
| | | CGCATCTGGGCTGA | | | | | | |
| | | CAGGTGCAGAATTG | | | | | | |
| | | GAGGTCATTTGGGG | | | | | | |
| | | GCTACCCCGTTCTG | | | | | | |
| | | TCCCGAGTATGCTC | | | | | | |
| | | TCGGCCCTGCTCAG | | | | | | |
| | | GCCAAGGGGAACCC | | | | | | |
| | | TGAGAGCAGCTTCA | | | | | | |
| | | ATGATGAGAACC | | | | | | |
| Template_M | CYP2D6_ | CCTGGGTCTACCTG | gblock-500 bp | | | | 100000 | 100000 |
| | M_T3 | GAGATGGCTGGGG | | | | | | |
| | | CCTGAGACTTGTCC | | | | | | |
| | | AGGTGAACGCAGAG | | | | | | |
| | | CACAGGAGGGATTG | | | | | | |
| | | AGACCCCGTTCTGT | | | | | | |
| | | CTGGTGTAGGTGCT | | | | | | |
| | | GAATGCTGTCCCCG | | | | | | |
| | | TCCTCCTGCATATC | | | | | | |
| | | CCAGCGCTGGCTGG | | | | | | |
| | | CAAGGTCCTACGCT | | | | | | |
| | | TCCAAAAGGCTTTC | | | | | | |
| | | CTGACCCAGCTGGA | | | | | | |
| | | TGAGCTGCTAACTG | | | | | | |
| | | AGCACGGATGACCT | | | | | | |
| | | GGGACCCAGCCCA | | | | | | |
| | | GCCCCCCCCGAGAC | | | | | | |
| | | CTGACTGAGGCCTT | | | | | | |
| | | CCTGGCAGAGATGG | | | | | | |
| | | AGGTGAGAGTGGCT | | | | | | |
| | | GCCACGGTGGGGG | | | | | | |
| | | GCAAGGGTGGTGG | | | | | | |
| | | GTTGAGCGTCCCAG | | | | | | |
| | | GAGGAATGAGGGGA | | | | | | |
| | | GGCTGGGCAAAAGG | | | | | | |
| | | TTGGACCAGTGCAT | | | | | | |
| | | CACCCGGCGAGCC | | | | | | |
| | | GCATCTGGGCTGAC | | | | | | |
| | | AGGTGCAGAATTGG | | | | | | |
| | | AGGTCATTTGGGGG | | | | | | |
| | | CTACCCCGTTCTGT | | | | | | |
| | | CCCGAGTATGCTCT | | | | | | |
| | | CGGCCCTGCTCAGG | | | | | | |
| | | CCAAGGGGAACCCT | | | | | | |
| | | GAGAGCAGCTTCAA | | | | | | |
| | | TGATGAGAACC | | | | | | |
| HapMap_ | NA19143, | | | | | | | |
| Homo WT | NA21114 | | | | | | | |
| HapMap_ | NA12762 | | | | | | | |
| Hetero | | | | | | | | |
| HapMap_ | HG00111 | | | | | | | |
| Homo M | | | | | | | | |

TABLE 25
SNP5
(rs16947)

| | | | | | Conc | | | |
|---|---|---|---|---|---|---|---|---|
| | | | | | after | | | Amount |
| | | | | Measured | 10× | Per | Final | (nmole) |
| | | | | conc. | dilution | Reaction | conc. | per rx |
| Component | Name | Direction 5′-3′ | Specifications | (uM) | (uM) | (uL) | (uM) | (25 ul) |
| Master mix | SSO | NA | #1725285 | | | 12.5 | 1× | NA |
| | Advanced | | | | | | | |
| | Universal | | | | | | | |
| | Probes | | | | | | | |
| | Supermix | | | | | | | |
| Primer F | rs16947_ | 5′ | 19 bp | 92.34 | 9.234 | 0.3 | 0.111 | 0.00277 |
| | F1 | CCGTTCTGTCCCGA | (Tm = 59.5) | | | | | |
| | (SNP5_F1) | GTATG 3′ | | | | | | |
| Primer R | rs16947_ | 5′ | 18 bp | 91.92 | 9.192 | 0.3 | 0.110 | 0.00276 |
| | R1 | GGTCACCATCCCGG | (Tm = 60.8) | | | | | |
| | (SNP5_R1) | CAGA 3′ | | | | | | |
| Probe A | rs16947_ | RFAM/AGCCACCACTA | [6FAM]-20 bp- | 90.18 | 9.018 | 3 | 1.082 | 0.02705 |
| | P2_WT_R | 5′ 6- | [BHQ1] | | | | | |
| | (SNP5_P2_ | TGCGCAGGT/BHQ1 | (Tm = 62.5) | | | | | |
| | WT_R) | 3′ | | | | | | |
| Probe B | rs16947_ | 5′ | [HEX]-20 bp- | 96.65 | 9.665 | 2.5 | 0.967 | 0.02416 |
| | P2_M_R_ | HEX/AGCCACCACTA | [IBFQ] | | | | | |
| | HEX | TGCACAGGT/ | (Tm = 60.5) | | | | | |
| | (SNP5_ | IBFQ 3′ | | | | | | |
| | P2_M_R_ | | | | | | | |
| | HEX) | | | | | | | |
| Tris-EDTA | 1× Tris- | NA | 1^{st} Base | | | 4.40 | NA | NA |
| buffer | EDTA | | | | | | | |
| | (TE) | | | | | | | |
| | Buffer | | | | | | | |
| | with | | | | | | | |
| | reduced | | | | | | | |
| | EDTA, pH | | | | | | | |
| | 8.0, | | | | | | | |
| | Biotechno | | | | | | | |
| | logy | | | | | | | |
| | Grade, 1L | | | | | | | |
| | (#CUS- | | | | | | | |
| | 3022- | | | | | | | |
| | 1 × 1L) | | | | | | | |
| Template_ | CYP2D6_ | 5′ | gblock-500 bp | | | | 100000 | 100000 |
| WT | WT_T5 | GGCTACCCCGTTCT | | | | | | |
| | | GTCCCGAGTATGCT | | | | | | |
| | | CTCGGCCCTGCTCA | | | | | | |
| | | GGCCAAGGGGAAC | | | | | | |
| | | CCTGAGAGCAGCTT | | | | | | |
| | | CAATGATGAGAACC | | | | | | |
| | | TGCGCATAGTGGTG | | | | | | |
| | | GCTGACCTGTTCTC | | | | | | |
| | | TGCCGGGATGGTGA | | | | | | |
| | | CCACCTCGACCACG | | | | | | |
| | | CTGGCCTGGGGCCT | | | | | | |
| | | CCTGCTCATGATCC | | | | | | |
| | | TACATCCGGATGTG | | | | | | |
| | | CAGCGTGAGCCCAT | | | | | | |
| | | CTGGGAAACAGTGC | | | | | | |
| | | AGGGGCCGAGGGA | | | | | | |
| | | GGAAGGGTACAGGC | | | | | | |
| | | GGGGGCCCATGAAC | | | | | | |
| | | TTTGCTGGGACACC | | | | | | |
| | | CGGGGCTCCAAGCA | | | | | | |
| | | CAGGCTTGACCAGG | | | | | | |
| | | ATCCTGTAAGCCTG | | | | | | |
| | | ACCTCCTCCAACAT | | | | | | |
| | | AGGAGGCAAGAAGG | | | | | | |
| | | AGTGTCAGGGCCGG | | | | | | |
| | | ACCCCCTGGGTGCT | | | | | | |
| | | GACCCATTGTGGGG | | | | | | |
| | | ACGCATGTCTGTCC | | | | | | |
| | | AGGCCGTGTCCAAC | | | | | | |
| | | AGGAGATCGACGAC | | | | | | |
| | | GTGATAGGGCAGGT | | | | | | |
| | | GCGGCGACCAGAG | | | | | | |
| | | ATGGGTGACCAGGC | | | | | | |
| | | TCACATGCCCTACA | | | | | | |
| | | CCACTGCCGTGATT | | | | | | |
| | | CATGAGGTGCAG 3′ | | | | | | |
| Template_M | CYP2D6_ | 5′ | gblock-500 bp | | | | 100000 | 100000 |
| | M_T5 | GGCTACCCCGTTCT | | | | | | |
| | | GTCCCGAGTATGCT | | | | | | |
| | | CTCGGCCCTGCTCA | | | | | | |
| | | GGCCAAGGGGAAC | | | | | | |
| | | CCTGAGAGCAGCTT | | | | | | |
| | | CAATGATGAGAACC | | | | | | |
| | | TGTGCATAGTGGTG | | | | | | |
| | | GCTGACCTGTTCTC | | | | | | |
| | | TGCCGGGATGGTGA | | | | | | |
| | | CCACCTCGACCACG | | | | | | |
| | | CTGGCCTGGGGCCT | | | | | | |
| | | CCTGCTCATGATCC | | | | | | |
| | | TACATCCGGATGTG | | | | | | |
| | | CAGCGTGAGCCCAT | | | | | | |
| | | CTGGGAAACAGTGC | | | | | | |
| | | AGGGGCCGAGGGA | | | | | | |
| | | GAAAGGGTACAGGC | | | | | | |
| | | GGGGGCCCATGAAC | | | | | | |
| | | TTTGCTGGGACACC | | | | | | |
| | | CGGGGCTCCAAGCA | | | | | | |
| | | CAGGCTTGACCAGG | | | | | | |
| | | ATCCTGTAAGCCTG | | | | | | |
| | | ACCTCCTCCAACAT | | | | | | |
| | | AGGAGGCAAGAAGG | | | | | | |
| | | AGTGTCAGGGCCGG | | | | | | |
| | | ACCCCCTGGGTGCT | | | | | | |
| | | GACCCATTGTGGGG | | | | | | |
| | | ACGCATGTCTGTCC | | | | | | |
| | | AGGCCGTGTCCAAC | | | | | | |
| | | AGGAGATCGACGAC | | | | | | |
| | | ATGATAGGGCAGGT | | | | | | |
| | | GCGGCGACCAGAG | | | | | | |
| | | ATGGGTGACCAGGC | | | | | | |
| | | TCACATGCCCTACA | | | | | | |

| | |
|---|---|
| HapMap_ | NA12873, |
| Homo WT | NA12762 |
| HapMap_ | NA19143, |
| Hetero | HG01398 |
| HapMap_ | NA18861, |
| Homo M | NA21114 |

TABLE 26
SNP6
(rs28371725)

| | | | | | Conc | | | |
|---|---|---|---|---|---|---|---|---|
| | | | | | after | | | Amount |
| | | | | Measured | 10× | Per | Final | (nmole) |
| | | | | conc. | dilution | Reaction | conc. | per rx |
| Component | Name | Direction 5′-3′ | Specifications | (uM) | (uM) | (uL) | (uM) | (25 ul) |
| Master mix | SSO | NA | #1725285 | | | 12.5 | 1× | NA |
| | Advanced | | | | | | | |
| | Universal | | | | | | | |
| | Probes | | | | | | | |
| | Supermix | | | | | | | |
| Primer F | rs28371725_ | 5′ | 19 bp | 88.89 | 8.889 | 1 | 0.356 | 0.00889 |
| | F2 | CGTGAGCCCATCTG | (Tm = 59.5) | | | | | |
| | (SNP6_F2) | GGAAA 3′ | | | | | | |
| Primer R | rs28371725_ | 5′ | 19 bp | 90.78 | 9.078 | 0.5 | 0.182 | 0.00454 |
| | R4 | GAGGTCAGGCTTAC | (Tm = 57.5) | | | | | |
| | (SNP6_R4) | AGGAT 3′ | | | | | | |
| Probe A | rs28371725_ | 5′ 6- | [6FAM]-19 bp- | 91.70 | 9.170 | 1.5 | 0.550 | 0.01376 |
| | P4_WT_F | FAM/AGGGAGGAAG | [BHQ1] | | | | | |
| | (SNP6_ | GGTACAGGC/BHQ1 | (Tm = 61.6) | | | | | |
| | P4_W_F) | 3′ | | | | | | |
| Probe B | rs28371725_ | 5′ | [HEX]-19 bp- | 87.19 | 8.719 | 1.5 | 0.523 | 0.01308 |
| | P3_M_F_ | HEX/AGGGAGAAAG | [IBFQ] | | | | | |
| | HEX | GGTACAGGC/IBFQ 3′ | (Tm = 59.5) | | | | | |
| | (SNP6_P3_ | | | | | | | |
| | M_F_HEX) | | | | | | | |
| Tris-EDTA | 1× Tris- | NA | 1^{st} Base | | | 6.00 | NA | NA |
| buffer | EDTA | | | | | | | |
| | (TE) | | | | | | | |
| | Buffer | | | | | | | |
| | with | | | | | | | |
| | reduced | | | | | | | |
| | EDTA, pH | | | | | | | |
| | 8.0, | | | | | | | |
| | Biotechno | | | | | | | |
| | logy | | | | | | | |
| | Grade, 1L | | | | | | | |
| | K#CUS- | | | | | | | |
| | 3022- | | | | | | | |
| | 1 × 1L) | | | | | | | |
| Template_ | CYP2D6_ | 5′ | gblock-500 bp | | | | 100000 | 100000 |
| WT | WT_T5 | GGCTACCCCGTTCT | | | | | | |
| | | GTCCCGAGTATGCT | | | | | | |
| | | CTCGGCCCTGCTCA | | | | | | |
| | | GGCCAAGGGGAAC | | | | | | |
| | | CCTGAGAGCAGCTT | | | | | | |
| | | CAATGATGAGAACC | | | | | | |
| | | TGCGCATAGTGGTG | | | | | | |
| | | GCTGACCTGTTCTC | | | | | | |
| | | TGCCGGGATGGTGA | | | | | | |
| | | CCACCTCGACCACG | | | | | | |
| | | CTGGCCTGGGGCCT | | | | | | |
| | | CCTGCTCATGATCC | | | | | | |
| | | TACATCCGGATGTG | | | | | | |
| | | CAGCGTGAGCCCAT | | | | | | |
| | | CTGGGAAACAGTGC | | | | | | |
| | | AGGGGCCGAGGGA | | | | | | |
| | | GGAAGGGTACAGGC | | | | | | |
| | | GGGGGCCCATGAAC | | | | | | |
| | | TTTGCTGGGACACC | | | | | | |
| | | CGGGGCTCCAAGCA | | | | | | |
| | | CAGGCTTGACCAGG | | | | | | |
| | | ATCCTGTAAGCCTG | | | | | | |
| | | ACCTCCTCCAACAT | | | | | | |
| | | AGGAGGCAAGAAGG | | | | | | |
| | | AGTGTCAGGGCCGG | | | | | | |
| | | ACCCCCTGGGTGCT | | | | | | |
| | | GACCCATTGTGGGG | | | | | | |
| | | ACGCATGTCTGTCC | | | | | | |
| | | AGGCCGTGTCCAAC | | | | | | |
| | | AGGAGATCGACGAC | | | | | | |
| | | GTGATAGGGCAGGT | | | | | | |
| | | GCGGCGACCAGAG | | | | | | |
| | | ATGGGTGACCAGGC | | | | | | |
| | | TCACATGCCCTACA | | | | | | |
| | | CCACTGCCGTGATT | | | | | | |
| | | CATGAGGTGCAG 3′ | | | | | | |
| Template_M | CYP2D6_ | 5′ | gblock-500 bp | | | | 100000 | 100000 |
| | M_T5 | GGCTACCCCGTTCT | | | | | | |
| | | GTCCCGAGTATGCT | | | | | | |
| | | CTCGGCCCTGCTCA | | | | | | |
| | | GGCCAAGGGGAAC | | | | | | |
| | | CCTGAGAGCAGCTT | | | | | | |
| | | CAATGATGAGAACC | | | | | | |
| | | TGTGCATAGTGGTG | | | | | | |
| | | GCTGACCTGTTCTC | | | | | | |
| | | TGCCGGGATGGTGA | | | | | | |
| | | CCACCTCGACCACG | | | | | | |
| | | CTGGCCTGGGGCCT | | | | | | |
| | | CCTGCTCATGATCC | | | | | | |
| | | TACATCCGGATGTG | | | | | | |
| | | CAGCGTGAGCCCAT | | | | | | |
| | | CTGGGAAACAGTGC | | | | | | |
| | | AGGGGCCGAGGGA | | | | | | |
| | | GAAAGGGTACAGGC | | | | | | |
| | | IGGGGGCCCATGAAC | | | | | | |
| | | TTTGCTGGGACACC | | | | | | |
| | | CGGGGCTCCAAGCA | | | | | | |
| | | CAGGCTTGACCAGG | | | | | | |
| | | ATCCTGTAAGCCTG | | | | | | |
| | | ACCTCCTCCAACAT | | | | | | |
| | | AGGAGGCAAGAAGG | | | | | | |
| | | AGTGTCAGGGCCGG | | | | | | |
| | | ACCCCCTGGGTGCT | | | | | | |
| | | GACCCATTGTGGGG | | | | | | |
| | | ACGCATGTCTGTCC | | | | | | |
| | | AGGCCGTGTCCAAC | | | | | | |
| | | AGGAGATCGACGAC | | | | | | |
| | | ATGATAGGGCAGGT | | | | | | |
| | | GCGGCGACCAGAG | | | | | | |
| | | ATGGGTGACCAGGC | | | | | | |
| | | TCACATGCCCTACA | | | | | | |
| | | CCACTGCCGTGATT | | | | | | |
| | | CATGAGGTGCAG 3′ | | | | | | |

| | |
|---|---|
| HapMap_ | HG00358, |
| Homo WT | NA12873 |
| HapMap_ | HG02684, |
| Hetero | NA12006 |
| HapMap_ | NA21114 |
| Homo M | |

TABLE 27
SNP7
(rs1135840)

| | | | | | Conc | | | |
|---|---|---|---|---|---|---|---|---|
| | | | | | after | | | Amount |
| | | | | Measured | 10× | Per | Final | (nmole) |
| | | | | conc. | dilution | Reaction | conc | per rx |
| Component | Name | Direction 5′-3′ | Specifications | (uM) | (uM) | (uL) | (uM) | (25 ul) |
| Master mix | SSO | NA | #1725285 | | | 12.5 | 1× | NA |
| | Advanced | | | | | | | |
| | Universal | | | | | | | |
| | Probes | | | | | | | |
| | Supermix | | | | | | | |
| Primer F | rs1135840_ | 5′ | 21 bp | 93.09 | 9.309 | 0.5 | 0.186 | 0.00465 |
| | F1 | ACCATGGTGTCTTT | (Tm = 59.5) | | | | | |
| | (SNP7_F1) | GCTTTCC 3′ | | | | | | |
| Primer R | rs1135840_ | 5′ | 17 bp | 89.35 | 8.935 | 0.5 | 0.179 | 0.00447 |
| | R2 | GTGAGCAGGGGAC | (Tm = 59.8) | | | | | |
| | (SNP7_R2) | CCGA 3′ | | | | | | |
| Probe A | rs1135840_ | 5′ 6- | [6FAM]-20 bp- | 91.18 | 9.118 | 0.25 | 0.091 | 0.00228 |
| | P1_WT_F | FAM/TGGTGAGCCC | [BHQ1] | | | | | |
| | (SNP7_P1_ | ATCCCCCTAT/BHQ1 | (Tm = 62.5) | | | | | |
| | WT_F) | 3′ | | | | | | |
| Probe B | rs1135840_ | 5′ | [HEX]-20 bp- | 109.93 | 10.993 | 0.5 | 0.220 | 0.00550 |
| | P1_M_ | HEX/TGGTGACCCCA | [IBFQ] | | | | | |
| | F_HEX | TCCCCCTAT/IBFQ 3′ | (Tm = 62.5) | | | | | |
| | (SNP7_P1_ | | | | | | | |
| | M_F_HEX) | | | | | | | |
| Tris-EDTA | 1 × Tris- | NA | 1^{st} Base | | | 8.75 | NA | NA |
| buffer | EDTA | | | | | | | |
| | (TE) | | | | | | | |
| | Buffer | | | | | | | |
| | with | | | | | | | |
| | reduced | | | | | | | |
| | EDTA, pH | | | | | | | |
| | 8.0, | | | | | | | |
| | Biotechno | | | | | | | |
| | logy | | | | | | | |
| | Grade, 1L | | | | | | | |
| | #CUS- | | | | | | | |
| | 3022- | | | | | | | |
| | 1 × 1L) | | | | | | | |
| Template_ | CYP2D6_ | 5′ | gblock-500 bp | | | | 100000 | 100000 |
| WT | WT_T4 | CTGGGAGAAGCCCT | | | | | | |
| | | TCCGCTTCCACCCC | | | | | | |
| | | GAACACTTCCTGGA | | | | | | |
| | | TGCCCAGGGCCACT | | | | | | |
| | | TTGTGAAGCCGGAG | | | | | | |
| | | GCCTTCCTGCCTTT | | | | | | |
| | | CTCAGCAGGTGCCT | | | | | | |
| | | GTGGGGAGCCCGG | | | | | | |
| | | CTCCCTGTCCCCTT | | | | | | |
| | | CCGTGGAGTCTTGC | | | | | | |
| | | AGGGGTATCACCCA | | | | | | |
| | | GGAGCCAGGCTCAC | | | | | | |
| | | TGACGCCCCTCCCC | | | | | | |
| | | TCCCCACAGGCCGC | | | | | | |
| | | CGTGCATGCCTCGG | | | | | | |
| | | GGAGCCCCTGGCC | | | | | | |
| | | CGCATGGAGCTCTT | | | | | | |
| | | CCTCTTCTTCACCTC | | | | | | |
| | | CCTGCTGCAGCACT | | | | | | |
| | | TCAGCTTCTCGGTG | | | | | | |
| | | CCCACTGGACAGCC | | | | | | |
| | | CCGGCCCAGCCACC | | | | | | |
| | | ATGGTGTCTTTGCTT | | | | | | |
| | | TCCTGGTGAGCCCA | | | | | | |
| | | TCCCCCTATGAGCT | | | | | | |
| | | TTGTGCTGTGCCCC | | | | | | |
| | | GCTAGAATGGGGTA | | | | | | |
| | | CCTAGTCCCCAGCC | | | | | | |
| | | TGCTCCCTAGCCAG | | | | | | |
| | | AGGCTCTAATGTAC | | | | | | |
| | | AATAAAGCAATGTG | | | | | | |
| | | GTAGTTCCAACTCG | | | | | | |
| | | GGTCCCCTGCTCAC | | | | | | |
| | | GCCCTCGTTGGGAT | | | | | | |
| | | CATCCTCCTCAGGG | | | | | | |
| | | CAACCCCACC 3′ | | | | | | |
| Template_M | CYP2D6_ | 5′ | gblock-500 bp | | | | 10000 | 100000 |
| | M_T4 | CTGGGAGAAGCCCT | | | | | 0 | |
| | | TCCGCTTCCACCCC | | | | | | |
| | | GAACACTTCCTGGA | | | | | | |
| | | TGCCCAGGGCCACT | | | | | | |
| | | TTGTGAAGCCGGAG | | | | | | |
| | | GCCTTCCTGCCTTT | | | | | | |
| | | CTCAGCAGGTGCCT | | | | | | |
| | | GTGGGGAGCCCGG | | | | | | |
| | | CTCCCTGTCCCCTT | | | | | | |
| | | CCGTGGAGTCTTGC | | | | | | |
| | | AGGGGTATCACCCA | | | | | | |
| | | GGAGCCAGGCTCAC | | | | | | |
| | | TGACGCCCCTCCCC | | | | | | |
| | | TCCCCACAGGCCAC | | | | | | |
| | | CGTGCATGCCTCGG | | | | | | |
| | | GGAGCCCCTGGCC | | | | | | |
| | | CGCATGGAGCTCTT | | | | | | |
| | | CCTCTTCTTCACCTC | | | | | | |
| | | CCTGCTGCAGCACT | | | | | | |
| | | TCAGCTTCTCGGTG | | | | | | |
| | | CCCACTGGACAGCC | | | | | | |
| | | CCGGCCCAGCCACC | | | | | | |
| | | ATGGTGTCTTTGCTT | | | | | | |
| | | TCCTGGTGACCCCA | | | | | | |
| | | TCCCCCTATGAGCT | | | | | | |
| | | TTGTGCTGTGCCCC | | | | | | |
| | | GCTAGAATGGGGTA | | | | | | |
| | | CCTAGTCCCCAGCC | | | | | | |
| | | TGCTCCCTAGCCAG | | | | | | |
| | | AGGCTCTAATGTAC | | | | | | |
| | | AATAAAGCAATGTG | | | | | | |
| | | GTAGTTCCAACTCG | | | | | | |
| | | GGTCCCCTGCTCAC | | | | | | |
| | | GCCCTCGTTGGGAT | | | | | | |
| | | CATCCTCCTCAGGG | | | | | | |
| | | CAACCCCACC 3′ | | | | | | |
| HapMap_ | NA12762, | | | | | | | |
| Homo WT | HG00111 | | | | | | | |
| HapMap_ | NA12872, | | | | | | | |
| Hetero | NA19201, | | | | | | | |

| | |
|---|---|
| | NA18990, |
| | NA11830, |
| | HG02684 |
| HapMap_ | NA18861, |
| Homo M | NA21114 |

TABLE 28
SNP8
(rs769258)

| | | | | | Conc | | | |
|---|---|---|---|---|---|---|---|---|
| | | | | | after | | | Amount |
| | | | | Measured | 10× | Per | Final | (nmole) |
| | | | | conc. | dilution | Reaction | conc | per rx |
| Component | Name | Direction 5′-3′ | Specifications | (uM) | (uM) | (uL) | (uM) | (25 ul) |
| Master mix | SSO | NA | #1725285 | | | 12.5 | 1× | NA |
| | Advanced | | | | | | | |
| | Universal | | | | | | | |
| | Probes | | | | | | | |
| | Supermix | | | | | | | |
| Primer F | rs769258_ | 5′ | 18 bp | 97.89 | 9.789 | 0.4 | 0.157 | 0.00392 |
| | F2 | GTGTCCAGAGGAGC | (Tm = 58.4) | | | | | |
| | (SNP8_F2) | CCAT 3′ | | | | | | |
| Primer R | rs769258_ | 5′ | 17 bp | 94.50 | 9.450 | 0.4 | 0.151 | 0.00378 |
| | R3 | GTGGCAGGGGGCTT | (Tm = 59.8) | | | | | |
| | (SNP8_R3) | GGT 3′ | | | | | | |
| Probe A | rs769258_ | 5′ 6- | [6FAM]-20 bp- | 91.86 | 9.186 | 3 | 1.102 | 0.02756 |
| | P2_WT_ | FAM/TGGTGCCCCT | [BHQ1] | | | | | |
| | F | GGCCGTGATA/BHQ1 | (Tm = 64.6) | | | | | |
| | (SNP8_P2_ | 3′ | | | | | | |
| | WT_F) | | | | | | | |
| Probe B | rs769258 | 5′ | [HEX]-20 bp- | 104.80 | 10.480 | 3.5 | 1.467 | 0.03668 |
| | P2_M_F | HEX/TGGTGCCCCTG | [IBFQ] | | | | | |
| | HEX | GCCATGATA/IBFQ 3′ | (Tm = 62.5) | | | | | |
| | (SNP8_P2_ | | | | | | | |
| | M_F_HEX) | | | | | | | |
| Tris-EDTA | 1× Tris- | NA | 1^{st} Base | | | 3.20 | NA | NA |
| buffer | EDTA | | | | | | | |
| | (TE) | | | | | | | |
| | Buffer | | | | | | | |
| | with | | | | | | | |
| | reduced | | | | | | | |
| | EDTA, pH | | | | | | | |
| | 8.0, | | | | | | | |
| | Biotechno | | | | | | | |
| | logy | | | | | | | |
| | Grade, 1L | | | | | | | |
| | (#CUS- | | | | | | | |
| | 3022- | | | | | | | |
| | 1 × 1L) | | | | | | | |
| Template_ | T1_WT_ | GAGTGTCCTGCCTG | gblock-335 bp | | | | 100000 | 100000 |
| WT | Extended | GTCCTCTGTGCCTG | | | | | | |
| | for R5 | GTGGGGTGGGGGT | | | | | | |
| | | GCCAGGTGTGTCCA | | | | | | |
| | | GAGGAGCCCATTTG | | | | | | |
| | | GTAGTGAGGCAGGT | | | | | | |
| | | ATGGGGCTAGAAGC | | | | | | |
| | | ACTGGTGCCCCTGG | | | | | | |
| | | CCGTGATAGTGGCC | | | | | | |
| | | ATCTTCCTGCTCCT | | | | | | |
| | | GGTGGACCTGATGC | | | | | | |
| | | ACCGGCGCCAACGC | | | | | | |
| | | TGGGCTGCACGCTA | | | | | | |
| | | CCCACCAGGCCCCC | | | | | | |
| | | TGCCACTGCCCGGG | | | | | | |
| | | CTGGGCAACCTGCT | | | | | | |
| | | GCATGTGGACTTCC | | | | | | |
| | | AGAACACACCATAC | | | | | | |
| | | TGCTTCGACCAGGT | | | | | | |
| | | GAGGGAGGAGGTC | | | | | | |
| | | CTGGAGGGCGGCA | | | | | | |
| | | GAGGTGCTGAGGCT | | | | | | |
| | | CCCCTACCAGAAGC | | | | | | |
| | | AAACATGGATGGTG | | | | | | |
| | | GG | | | | | | |
| Template_M | T1_MT_ | GAGTGTCCTGCCTG | gblock-335bp | | | | 100000 | 100000 |
| | | GTCCTCTGTGCCTG | | | | | | |
| | Extended | GTGGGGTGGGGGT | | | | | | |
| | for R5 | GCCAGGTGTGTCCA | | | | | | |
| | | GAGGAGCCCATTTG | | | | | | |
| | | GTAGTGAGGCAGGT | | | | | | |
| | | ATGGGGCTAGAAGC | | | | | | |
| | | ACTGGTGCCCCTGG | | | | | | |
| | | CCATGATAGTGGCC | | | | | | |
| | | ATCTTCCTGCTCCT | | | | | | |
| | | GGTGGACCTGATGC | | | | | | |
| | | ACCGGCGCCAACGC | | | | | | |
| | | TGGGCTGCACGCTA | | | | | | |
| | | CTCACCAGGCCCCC | | | | | | |
| | | TGCCACTGCCCGGG | | | | | | |
| | | CTGGGCAACCTGCT | | | | | | |
| | | GCATGTGGACTTCC | | | | | | |
| | | AGAACACACCATAC | | | | | | |
| | | TGCTTCGACCAGGT | | | | | | |
| | | GAGGGAGGAGGTC | | | | | | |
| | | CTGGAGGGCGGCA | | | | | | |
| | | GAGGTGCTGAGGCT | | | | | | |
| | | CCCCTACCAGAAGC | | | | | | |
| | | AAACATGGATGGTG | | | | | | |
| | | GG | | | | | | |
| HapMap_ | NA19201, | | | | | | | |
| Homo WT | NA21114 | | | | | | | |
| HapMap_ | NA12827, | | | | | | | |
| Hetero | NA12872 | | | | | | | |
| HapMap_ | HG00358 | | | | | | | |
| Homo M | | | | | | | | |

TABLE 29
SNP9
(rs5030865)

| | | | | | Conc | | | |
|---|---|---|---|---|---|---|---|---|
| | | | | | after | | | Amount |
| | | | | Measured | 10× | Per | Final | (nmole) |
| | | | | conc. | dilution | Reaction | conc. | per rx |
| Component | Name | Direction 5′-3′ | Specifications | (UM) | (UM) | (uL) | (uM) | (25 ul) |
| Master mix | SSO | NA | #1725285 | | | 12.5 | 1× | NA |
| | Advanced | | | | | | | |
| | Universal | | | | | | | |
| | Probes | | | | | | | |
| | Supermix | | | | | | | |
| Primer F | rs5030865_ | 5′ | 18 bp | 94.19 | 9.419 | 0.5 | 0.188 | 0.00471 |
| | F2 | GTGTTCCTGGCGCG | (Tm = 58.4) | | | | | |
| | (SNP9_F2) | CTAT 3′ | | | | | | |
| Primer R | rs5030865_ | 5′ | 17 bp | 95.95 | 9.595 | 0.5 | 0.192 | 0.00480 |
| | R1 | GTAAGGGGTCGCCT | (Tm = 57.3) | | | | | |
| | (SNP9_R1) | TCC 3′ | | | | | | |
| Probe A | rs5030865_ | 5′ | [6FAM]-19 | 103.97 | 10.397 | 2.5 | 1.040 | 0.02599 |
| | P2b_WTF | FAM/TCGCCAACCAC | bp-[IBFQ] | | | | | |
| | (SNP9_ | TCCGGTGG/IBFQ 3′ | (Tm = 63.6) | | | | | |
| | P2b_WT_F) | | | | | | | |
| Probe B | rs5030865_ | 5′ | [HEX]-19 | 103.85 | 10.385 | 3 | 1.246 | 0.03115 |
| | P2b_MF | HEX/TCGCCAACCAC | bp-[IBFQ] | | | | | |
| | (SNP9_ | TCCAGTGG/IBFQ 3′ | (Tm = 61.6) | | | | | |
| | P2b_M_F) | | | | | | | |
| Probe C | rs5030865_ | 5′ | [CY5]-19 | 98.55 | 9.855 | 3 | 1.183 | 0.02956 |
| | P2b_*8_ | CY5/TCGCCAACCAC | bp-[IBRQ] | | | | | |
| | CY5 | TCCTGTGG/IBFQ 3′ | (Tm = 61.6) | | | | | |
| | (SNP9_ | | | | | | | |
| | P2b_*8_ | | | | | | | |
| | CY5) | | | | | | | |
| Tris-EDTA | 1× Tris- | NA | 1st Base | | | 1.00 | NA | NA |
| buffer | EDTA | | | | | | | |
| | (TE) | | | | | | | |
| | Buffer | | | | | | | |
| | with | | | | | | | |
| | reduced | | | | | | | |
| | EDTA, pH | | | | | | | |
| | 8.0, | | | | | | | |
| | Biotechno | | | | | | | |
| | logy | | | | | | | |
| | Grade, 1L | | | | | | | |
| | K#CUS- | | | | | | | |
| | 3022- | | | | | | | |
| | 1 × 1L) | | | | | | | |
| Template_WT | CYP2D6_ | GAGCCAGGGACTGC | gblock-500 bp | | | | 100000 | 100000 |
| | WT_T2 | GGGAGACCAGGGG | | | | | | |
| | | GAGCATAGGGTTGG | | | | | | |
| | | AGTGGGTGGTGGAT | | | | | | |
| | | GGTGGGGCTAATGC | | | | | | |
| | | CTTCATGGCCACGC | | | | | | |
| | | GCACGTGCCCGTCC | | | | | | |
| | | CACCCCCAGGGGTG | | | | | | |
| | | TTCCTGGCGCGCTA | | | | | | |
| | | TGGGCCCGCGTGG | | | | | | |
| | | CGCGAGCAGAGGC | | | | | | |
| | | GCTTCTCCGTGTCC | | | | | | |
| | | ACCTTGCGCAACTT | | | | | | |
| | | GGGCCTGGGCAAG | | | | | | |
| | | AAGTCGCTGGAGCA | | | | | | |
| | | GTGGGTGACCGAG | | | | | | |
| | | GAGGCCGCCTGCCT | | | | | | |
| | | TTGTGCCGCCTTCG | | | | | | |
| | | CCAACCACTCCGGT | | | | | | |
| | | GGGTGATGGGCAGA | | | | | | |
| | | AGGGCACAAAGCGG | | | | | | |
| | | GAACTGGGAAGGCG | | | | | | |
| | | GGGGACGGGGAAG | | | | | | |
| | | GCGACCCCTTACCC | | | | | | |
| | | GCATCTCCCACCCC | | | | | | |
| | | CAGGACGCCCCTTT | | | | | | |
| | | CGCCCCAACGGTCT | | | | | | |
| | | CTTGGACAAAGCCG | | | | | | |
| | | TGAGCAACGTGATC | | | | | | |
| | | GCCTCCCTCACCTG | | | | | | |
| | | CGGGCGCCGCTTC | | | | | | |
| | | GAGTACGACGACCC | | | | | | |
| | | TCGCTTCCTCAGGC | | | | | | |
| | | TGCTGGACCTAGCT | | | | | | |
| | | CAGGAGGGACTGAA | | | | | | |
| | | GGAGGAGTCGGGC | | | | | | |
| | | TTT | | | | | | |
| Template_M | CYP2D6M_T2 | GAGCCAGGGACTGC | gblock-500 bp | | | | 100000 | 100000 |
| (*14) | | GGGAGACCAGGGG | | | | | | |
| | | GAGCATAGGGTTGG | | | | | | |
| | | AGTGGGTGGTGGAT | | | | | | |
| | | GGTGGGGCTAATGC | | | | | | |
| | | CTTCATGGCCACGC | | | | | | |
| | | GCACGTGCCCGTCC | | | | | | |
| | | CACCCCCAGGGGTG | | | | | | |
| | | TTCCTGGCGCGCTA | | | | | | |
| | | TGGGCCCGCGTGG | | | | | | |
| | | CGCGAGCAGAGGC | | | | | | |
| | | GCTTCTCCGTGTCC | | | | | | |
| | | ACCTTGCGCAACTT | | | | | | |
| | | GGGCCTGGGCAAG | | | | | | |
| | | AAGTCGCTGGAGCA | | | | | | |
| | | GGGGTGACCGAGG | | | | | | |
| | | AGGCCGCCTGCCTT | | | | | | |
| | | TGTGCCGCCTTCGC | | | | | | |
| | | CAACCACTCCAGTG | | | | | | |
| | | GGTGATGGGCAGAA | | | | | | |
| | | GGGCACAAAGCGG | | | | | | |
| | | GAACTGGGAAGGCG | | | | | | |
| | | GGGGACGGGGAAG | | | | | | |
| | | GCGACCCCTTACCC | | | | | | |
| | | GCATCTCCCACCCC | | | | | | |
| | | CAAGACGCCCCTTT | | | | | | |
| | | CGCCCCAACGGTCT | | | | | | |
| | | CTTGGACAAAGCCG | | | | | | |
| | | TGAGCAACGTGATC | | | | | | |
| | | GCCTCCCTCACCTG | | | | | | |
| | | CGGGCGCCGCTTC | | | | | | |
| | | GAGTACGACGACCC | | | | | | |
| | | TCGCTTCCTCAGGC | | | | | | |
| | | TGCTGGACCTAGCT | | | | | | |
| | | CAGGAGGGACTGAA | | | | | | |
| | | GGAGGAGTCGGGC | | | | | | |
| | | TTT | | | | | | |
| Template_M | CYP2D6_ | GAGCCAGGGACTGC | gblock-498 bp | | | | 100000 | 100000 |
| (*8) | MT*8 | GGGAGACCAGGGG | | | | | | |
| | | GAGCATAGGGTTGG | | | | | | |
| | | AGTGGGTGGTGGAT | | | | | | |
| | | GGTGGGGCTAATGC | | | | | | |
| | | CTTCATGGCCACGC | | | | | | |
| | | GCACGTGCCCGTCC | | | | | | |
| | | CACCCCCAGGGGTG | | | | | | |
| | | TTCCTGGCGCGCTA | | | | | | |
| | | TGGGCCCGCGTGG | | | | | | |
| | | CGCGAGCAGAGGC | | | | | | |
| | | GCTTCTCCGTGTCC | | | | | | |
| | | ACCTTGCGCAACTT | | | | | | |
| | | GGGCCTGGGCAAG | | | | | | |
| | | AAGTCGCTGGAGCA | | | | | | |
| | | GGGGTGACCGAGG | | | | | | |
| | | AGGCCGCCTGCCTT | | | | | | |
| | | TGTGCCGCCTTCGC | | | | | | |
| | | CAACCACTCCTGTG | | | | | | |
| | | GGTGATGGGCAGAA | | | | | | |
| | | GGGCACAAAGCGG | | | | | | |
| | | GAACTGGGAAGGCG | | | | | | |
| | | GGGGACGGGGAAG | | | | | | |
| | | GCGACCCCTTACCC | | | | | | |
| | | GCATCTCCCACCCC | | | | | | |
| | | CAAGACGCCCCTTT | | | | | | |
| | | CGCCCCAACGGTCT | | | | | | |
| | | CTTGGACAAAGCCG | | | | | | |
| | | TGAGCAACGTGATC | | | | | | |
| | | GCCTCCCTCACCTG | | | | | | |
| | | CGGGCGCCGCTTC | | | | | | |
| | | GAGTACGACGACCC | | | | | | |
| | | TCGCTTCCTCAGGC | | | | | | |
| | | TGCTGGACCTAGCT | | | | | | |
| | | CAGGAGGGACTGAA | | | | | | |
| | | GGAGGAGTCGGGC | | | | | | |
| | | TTT | | | | | | |
| HapMap_ | NA06994, | | | | | | | |
| Homo WT | NA18990 | | | | | | | |
| HapMap_ | NA18552 | | | | | | | |
| Hetero | | | | | | | | |
| HapMap_ | N/A | | | | | | | |
| Homo M | | | | | | | | |

TABLE 30
SNP11
(rs5030656)

| | | | | | Conc | | | |
|---|---|---|---|---|---|---|---|---|
| | | | | | after | | Final | Amount |
| | | | | Measured | 10× | Per | conc. | (nmole) |
| | | | | conc. | dilution | Reaction | (uM or | per rx |
| Component | Name | Direction 5′-3′ | Specifications | (uM) | (uM) | (uL) | copies) | (25 ul) |
| Master mix | SSO | NA | #1725285 | | | 12.5 | 1× | NA |
| | Advanced | | | | | | | |
| | Universal | | | | | | | |
| | Probes | | | | | | | |
| | Supermix | | | | | | | |
| Primer F | rs5030656_ | 5′ | 19 bp (Tm = 59.5) | 93.09 | 9.309 | 1 | 0.372 | 0.0093 |
| | F1 | AGGCCTTCCTGGCA | | | | | | 1 |
| | (SNP11_ | GAGAT 3′ | | | | | | |
| | F1) | | | | | | | |
| Primer R | rs5030656_ | 5′ | 18 bp (Tm = 58.4) | 98.34 | 9.834 | 0.5 | 0.197 | 0.0049 |
| | R1 | TCATTCCTCCTGGG | | | | | | 2 |
| | (SNP11_ | ACGC 3′ | | | | | | |
| | R1) | | | | | | | |
| Probe A | rs5030656_ | 5′ | 22 bp (Tm = 62.1) | 82.94 | 8.294 | 2.5 | 0.829 | 0.0207 |
| | P2b_WT_F | FAM/AGAGATGGAG | | | | | | 3 |
| | (SNP11_ | AAGGTGAGAGTG/IB | | | | | | |
| | P2b_WT_F) | FQ 3′ | | | | | | |
| Probe B | rs5030656_ | 5′ | 19 bp (Tm = 57.5) | 87.79 | 8.779 | 4 | 1.405 | 0.0351 |
| | P2b_M_F | HEX/AGAGATGGAG | | | | | | 2 |
| | (SNP11_ | GTGAGAGTG/IBFQ 3′ | | | | | | |
| | P2b_M_F) | | | | | | | |
| Tris-EDTA | 1× Tris- | NA | 1st Base | | | 2.50 | NA | NA |
| buffer | EDTA | | | | | | | |
| | (TE) | | | | | | | |
| | Buffer | | | | | | | |
| | with | | | | | | | |
| | reduced | | | | | | | |
| | EDTA, pH | | | | | | | |
| | 8.0, | | | | | | | |
| | Biotechno | | | | | | | |
| | logy | | | | | | | |
| | Grade, 1L | | | | | | | |
| | (#CUS- | | | | | | | |
| | 3022- | | | | | | | |
| | 1 × 1L) | | | | | | | |
| Template_ | CYP2D6_ | CCTGGGTCTACCTG | gblock-500 bp | | | | 100000 | 100000 |
| WT | WT_T3 | GAGATGGCTGGGG | | | | | | |
| | | CCTGAGACTTGTCC | | | | | | |
| | | AGGTGAACGCAGAG | | | | | | |
| | | CACAGGAGGGATTG | | | | | | |
| | | AGACCCCGTTCTGT | | | | | | |
| | | CTGGTGTAGGTGCT | | | | | | |
| | | GAATGCTGTCCCCG | | | | | | |
| | | TCCTCCTGCATATC | | | | | | |
| | | CCAGCGCTGGCTGG | | | | | | |
| | | CAAGGTCCTACGCT | | | | | | |
| | | TCCAAAAGGCTTTC | | | | | | |
| | | CTGACCCAGCTGGA | | | | | | |
| | | TGAGCTGCTAACTG | | | | | | |
| | | AGCACAGGATGACC | | | | | | |
| | | TGGGACCCAGCCCA | | | | | | |
| | | GCCCCCCCGAGACC | | | | | | |
| | | TGACTGAGGCCTTC | | | | | | |
| | | CTGGCAGAGATGGA | | | | | | |
| | | GAAGGTGAGAGTGG | | | | | | |
| | | CTGCCACGGTGGG | | | | | | |
| | | GGGCAAGGGTGGT | | | | | | |
| | | GGGTTGAGCGTCCC | | | | | | |
| | | AGGAGGAATGAGGG | | | | | | |
| | | GAGGCTGGGCAAAA | | | | | | |
| | | GGTTGGACCAGTGC | | | | | | |
| | | ATCACCCGGCGAGC | | | | | | |
| | | CGCATCTGGGCTGA | | | | | | |
| | | 5′ | | | | | | |
| | | CAGGTGCAGAATTG | | | | | | |
| | | GAGGTCATTTGGGG | | | | | | |
| | | GCTACCCCGTTCTG | | | | | | |
| | | TCCCGAGTATGCTC | | | | | | |
| | | TCGGCCCTGCTCAG | | | | | | |
| | | GCCAAGGGGAACCC | | | | | | |
| | | TGAGAGCAGCTTCA | | | | | | |
| | | ATGATGAGAACC 3′ | | | | | | |
| Template_M | CYP2D6_ | 5′ | gblock-500 bp | | | | 100000 | 100000 |
| | M_T3 | CCTGGGTCTACCTG | | | | | | |
| | | GAGATGGCTGGGG | | | | | | |
| | | CCTGAGACTTGTCC | | | | | | |
| | | AGGTGAACGCAGAG | | | | | | |
| | | CACAGGAGGGATTG | | | | | | |
| | | AGACCCCGTTCTGT | | | | | | |
| | | CTGGTGTAGGTGCT | | | | | | |
| | | GAATGCTGTCCCCG | | | | | | |
| | | TCCTCCTGCATATC | | | | | | |
| | | CCAGCGCTGGCTGG | | | | | | |
| | | CAAGGTCCTACGCT | | | | | | |
| | | TCCAAAAGGCTTTC | | | | | | |
| | | CTGACCCAGCTGGA | | | | | | |
| | | TGAGCTGCTAACTG | | | | | | |
| | | AGCACGGATGACCT | | | | | | |
| | | GGGACCCAGCCCA | | | | | | |
| | | GCCCCCCCCGAGAC | | | | | | |
| | | CTGACTGAGGCCTT | | | | | | |
| | | CCTGGCAGAGATGG | | | | | | |
| | | AGGTGAGAGTGGCT | | | | | | |
| | | GCCACGGTGGGGG | | | | | | |
| | | GCAAGGGTGGTGG | | | | | | |
| | | GTTGAGCGTCCCAG | | | | | | |
| | | GAGGAATGAGGGGA | | | | | | |
| | | GGCTGGGCAAAAGG | | | | | | |
| | | TTGGACCAGTGCAT | | | | | | |
| | | CACCCGGCGAGCC | | | | | | |

| | | |
|---|---|---|
| | | GCATCTGGGCTGAC |
| | | AGGTGCAGAATTGG |
| | | AGGTCATTTGGGGG |
| | | CTACCCCGTTCTGT |
| | | CCCGAGTATGCTCT |
| | | CGGCCCTGCTCAGG |
| | | CCAAGGGGAACCCT |
| | | GAGAGCAGCTTCAA |
| | | TGATGAGAACC 3′ |
| HapMap_ | NA12762, | |
| Homo WT | HG00111 | |
| HapMap_ | NA12872 | |
| Hetero | | |
| HapMap_ | NA06989 | |
| Homo M | | |

TABLE 31
SNP12
(rs59421388)

| | | | | | Conc | | | |
|---|---|---|---|---|---|---|---|---|
| | | | | | after | | Final | Amount |
| | | | | Measured | 10× | Per | conc. | (nmole) |
| | | | | conc. | dilution | Reaction | (uM or | per rx |
| Component | Name | Direction 5′-3′ | Specifications | (uM) | (uM | (uL) | copies) | (25 ul) |
| Master mix | SSO | NA | #1725285 | | | 12.5 | 1× | NA |
| | Advanced | | | | | | | |
| | Universal | | | | | | | |
| | Probes | | | | | | | |
| | Supermix | | | | | | | |
| Primer F | rs59421388_ | 5′ | 19 bp (Tm = 57.5) | 99.23 | 9.923 | 0.5 | 0.198 | 0.0049 |
| | F2 | AGGATCCTGTAAGC | | | | | | 6 |
| | (SNP12_ | CTGAC 3′ | | | | | | |
| | F2) | | | | | | | |
| Primer R | rs59421388_ | 5 | 20 bp (Tm = 58.4) | 92.76 | 9.276 | 0.5 | 0.186 | 0.0046 |
| | R1 | ATGAATCACGGCAG | | | | | | 4 |
| | (SNP12_ | TGGTGT 3′ | | | | | | |
| | R1) | | | | | | | |
| Probe A | rs59421388_ | 5′ 6- | [6FAM]-21 bp- | 103.33 | 10.333 | 2 | 0.827 | 0.0206 |
| | P1_WT_F | FAM/ATCGACGACGT | [BHQ1] (Tm = 63.2) | | | | | 7 |
| | (SNP12 | GATAGGGCAG/BHQ1 | | | | | | |
| | P1_WT_F) | 3′ | | | | | | |
| Probe B | rs59421388_ | 5′ | [HEX]-21 bp- | 96.86 | 9.686 | 3 | 1.162 | 0.0290 |
| | P1_M_F_ | HEX/ATCGACGACAT | [IBFQ] (Tm = 61.2) | | | | | 6 |
| | HEX | GATAGGGCAG/IBFQ | | | | | | |
| | (SNP12_ | 3′ | | | | | | |
| | P1_M_F_ | | | | | | | |
| | HEX) | | | | | | | |
| Tris-EDTA | 1× Tris- | NA | 1st Base | | | 4.50 | NA | NA |
| buffer | EDTA | | | | | | | |
| | (TE) | | | | | | | |
| | Buffer | | | | | | | |
| | with | | | | | | | |
| | reduced | | | | | | | |
| | EDTA, pH | | | | | | | |
| | 8.0, | | | | | | | |
| | Biotechno | | | | | | | |
| | logy | | | | | | | |
| | Grade, 1L | | | | | | | |
| | #CUS- | | | | | | | |
| | 3022- | | | | | | | |
| | 1 × 1L) | | | | | | | |
| Template_ | CYP2D6_ | 5′ | gblock-500 bp | | | | 100000 | 100000 |
| WT | WT_T5 | GGCTACCCCGTTCT | | | | | | |
| | | GTCCCGAGTATGCT | | | | | | |
| | | CTCGGCCCTGCTCA | | | | | | |
| | | GGCCAAGGGGAAC | | | | | | |
| | | CCTGAGAGCAGCTT | | | | | | |
| | | CAATGATGAGAACC | | | | | | |
| | | TGCGCATAGTGGTG | | | | | | |
| | | GCTGACCTGTTCTC | | | | | | |
| | | TGCCGGGATGGTGA | | | | | | |
| | | CCACCTCGACCACG | | | | | | |
| | | CTGGCCTGGGGCCT | | | | | | |
| | | CCTGCTCATGATCC | | | | | | |
| | | TACATCCGGATGTG | | | | | | |
| | | CAGCGTGAGCCCAT | | | | | | |
| | | CTGGGAAACAGTGC | | | | | | |
| | | AGGGGCCGAGGGA | | | | | | |
| | | GGAAGGGTACAGGC | | | | | | |
| | | GGGGGCCCATGAAC | | | | | | |
| | | TTTGCTGGGACACC | | | | | | |
| | | CGGGGCTCCAAGCA | | | | | | |
| | | CAGGCTTGACCAGG | | | | | | |
| | | ATCCTGTAAGCCTG | | | | | | |
| | | ACCTCCTCCAACAT | | | | | | |
| | | AGGAGGCAAGAAGG | | | | | | |
| | | AGTGTCAGGGCCGG | | | | | | |
| | | ACCCCCTGGGTGCT | | | | | | |
| | | GACCCATTGTGGGG | | | | | | |
| | | ACGCATGTCTGTCC | | | | | | |
| | | AGGCCGTGTCCAAC | | | | | | |
| | | AGGAGATCGACGAC | | | | | | |
| | | GTGATAGGGCAGGT | | | | | | |
| | | GCGGCGACCAGAG | | | | | | |
| | | ATGGGTGACCAGGC | | | | | | |
| | | TCACATGCCCTACA | | | | | | |
| | | CCACTGCCGTGATT | | | | | | |
| | | CATGAGGTGCAG 3′ | | | | | | |
| Template_M | CYP2D6_ | 5′ | gblock-500 bp | | | | 100000 | 100000 |
| | M_T5 | GGCTACCCCGTTCT | | | | | | |
| | | GTCCCGAGTATGCT | | | | | | |
| | | CTCGGCCCTGCTCA | | | | | | |
| | | GGCCAAGGGGAAC | | | | | | |
| | | CCTGAGAGCAGCTT | | | | | | |
| | | CAATGATGAGAACC | | | | | | |
| | | TGTGCATAGTGGTG | | | | | | |
| | | GCTGACCTGTTCTC | | | | | | |
| | | TGCCGGGATGGTGA | | | | | | |
| | | CCACCTCGACCACG | | | | | | |
| | | CTGGCCTGGGGCCT | | | | | | |
| | | CCTGCTCATGATCC | | | | | | |
| | | TACATCCGGATGTG | | | | | | |
| | | CAGCGTGAGCCCAT | | | | | | |
| | | CTGGGAAACAGTGC | | | | | | |
| | | AGGGGCCGAGGGA | | | | | | |
| | | GAAAGGGTACAGGC | | | | | | |
| | | GGGGGCCCATGAAC | | | | | | |
| | | TTTGCTGGGACACC | | | | | | |
| | | CGGGGCTCCAAGCA | | | | | | |
| | | CAGGCTTGACCAGG | | | | | | |
| | | ATCCTGTAAGCCTG | | | | | | |
| | | ACCTCCTCCAACAT | | | | | | |
| | | AGGAGGCAAGAAGG | | | | | | |
| | | AGTGTCAGGGCCGG | | | | | | |
| | | ACCCCCTGGGTGCT | | | | | | |
| | | GACCCATTGTGGGG | | | | | | |
| | | ACGCATGTCTGTCC | | | | | | |
| | | AGGCCGTGTCCAAC | | | | | | |
| | | AGGAGATCGACGAC | | | | | | |
| | | ATGATAGGGCAGGT | | | | | | |
| | | GCGGCGACCAGAG | | | | | | |
| | | ATGGGTGACCAGGC | | | | | | |
| | | TCACATGCCCTACA | | | | | | |
| | | CCACTGCCGTGATT | | | | | | |
| | | CATGAGGTGCAG 3′ | | | | | | |
| HapMap_ | NA12762, | | | | | | | |
| Homo WT | NA19143 | | | | | | | |
| HapMap_ | NA19393, | | | | | | | |
| Hetero | NA19130, | | | | | | | |
| | NA19332 | | | | | | | |
| HapMap_ | NA18861 | | | | | | | |
| Homo M | | | | | | | | |

TABLE 32
SNP13
(rs267608319)

| | | | | | Conc | | | |
|---|---|---|---|---|---|---|---|---|
| | | | | | after | | | Amount |
| | | | | Measured | 10× | Per | Final | (nmole) |
| | | | | conc. | dilution | Reaction | conc. | per rx |
| Component | Name | Direction 5′-3′ | Specifications | (uM) | (uM) | (uL) | (uM) | (25 ul) |
| Master mix | SSO | NA | #1725285 | | | 12.5 | 1× | NA |
| | Advanced | | | | | | | |
| | Universal | | | | | | | |
| | Probes | | | | | | | |
| | Supermix | | | | | | | |
| Primer F | rs267608319_ | 5′ | 7 | 94.43 | 9.443 | 0.1 | 0.038 | 0.00094 |
| | F2 | AGGGCCACTTTGTG | | | | | | |
| | (SNP13_ | AAGCC 3′ | | | | | | |
| | F2) | | | | | | | |
| Primer R | rs267608319_ | 5′ | 21 bp (Tm = 59.5) | 90.58 | 9.058 | 0.5 | 0.181 | 0.00453 |
| | R2 | CAGGAAAGCAAAGA | | | | | | |
| | (SNP13_ | CACCATG 3′ | | | | | | |
| | R2) | | | | | | | |
| Probe A | rs267608319_ | 5′ | [6FAM]-18 bp- | 89.76 | 8.976 | 1.75 | 0.628 | 0.01571 |
| | P3_WT_F | 6-FAM/CACAGGCCGC | [BHQ1] (Tm = 62.9) | | | | | |
| | (SNP13_ | CGTGCATG/BHQ13′ | | | | | | |
| | P3_WT_F) | | | | | | | |
| Probe B | rs267608319_ | 5′ | [HEX]-19 bp- | 97.62 | 9.762 | 2.25 | 0.879 | 0.02196 |
| | P3_ | HEX/CCACAGGCCA | [IBFQ] (Tm = 63.6) | | | | | |
| | M_F_HEX | CCGTGCATG/IBFQ 3′ | | | | | | |
| | (SNP13_ | | | | | | | |
| | P3_M_F_ | | | | | | | |
| | HEX) | | | | | | | |
| Tris-EDTA | 1× Tris- | NA | 1st Base | | | 5.90 | NA | NA |
| buffer | EDTA | | | | | | | |
| | (TE) | | | | | | | |
| | Buffer | | | | | | | |
| | with | | | | | | | |
| | reduced | | | | | | | |
| | EDTA, pH | | | | | | | |
| | 8.0, | | | | | | | |
| | Biotechnology | | | | | | | |
| | Grade, 1L | | | | | | | |
| | #CUS- | | | | | | | |
| | 3022- | | | | | | | |
| | 1 × 1L) | | | | | | | |
| Template_ | CYP2D6_ | 5′ | gblock-500 bp | | | | 100000 | 100000 |
| WT | WT_T4 | CTGGGAGAAGCCCT | | | | | | |
| | | TCCGCTTCCACCCC | | | | | | |
| | | GAACACTTCCTGGA | | | | | | |
| | | TGCCCAGGGCCACT | | | | | | |
| | | TTGTGAAGCCGGAG | | | | | | |
| | | GCCTTCCTGCCTTT | | | | | | |
| | | CTCAGCAGGTGCCT | | | | | | |
| | | GTGGGGAGCCCGG | | | | | | |
| | | CTCCCTGTCCCCTT | | | | | | |
| | | CCGTGGAGTCTTGC | | | | | | |
| | | AGGGGTATCACCCA | | | | | | |
| | | GGAGCCAGGCTCAC | | | | | | |
| | | TGACGCCCCTCCCC | | | | | | |
| | | TCCCCACAGGCCGC | | | | | | |
| | | CGTGCATGCCTCGG | | | | | | |
| | | GGAGCCCCTGGCC | | | | | | |
| | | CGCATGGAGCTCTT | | | | | | |
| | | CCTCTTCTTCACCTC | | | | | | |
| | | CCTGCTGCAGCACT | | | | | | |
| | | TCAGCTTCTCGGTG | | | | | | |
| | | CCCACTGGACAGCC | | | | | | |
| | | CCGGCCCAGCCACC | | | | | | |
| | | ATGGTGTCTTTGCTT | | | | | | |
| | | TCCTGGTGAGCCCA | | | | | | |
| | | TCCCCCTATGAGCT | | | | | | |
| | | TTGTGCTGTGCCCC | | | | | | |
| | | GCTAGAATGGGGTA | | | | | | |
| | | CCTAGTCCCCAGCC | | | | | | |
| | | TGCTCCCTAGCCAG | | | | | | |
| | | AGGCTCTAATGTAC | | | | | | |
| | | AATAAAGCAATGTG | | | | | | |
| | | GTAGTTCCAACTCG | | | | | | |
| | | GGTCCCCTGCTCAC | | | | | | |
| | | GCCCTCGTTGGGAT | | | | | | |
| | | CATCCTCCTCAGGG | | | | | | |
| | | CAACCCCACC 3′ | | | | | | |
| Template_M | CYP2D6_ | 5′ | gblock-500 bp | | | | 100000 | 100000 |
| | M_T4 | CTGGGAGAAGCCCT | | | | | | |
| | | TCCGCTTCCACCCC | | | | | | |
| | | GAACACTTCCTGGA | | | | | | |
| | | TGCCCAGGGCCACT | | | | | | |
| | | TTGTGAAGCCGGAG | | | | | | |
| | | GCCTTCCTGCCTTT | | | | | | |
| | | CTCAGCAGGTGCCT | | | | | | |
| | | GTGGGGAGCCCGG | | | | | | |
| | | CTCCCTGTCCCCTT | | | | | | |
| | | CCGTGGAGTCTTGC | | | | | | |
| | | AGGGGTATCACCCA | | | | | | |
| | | GGAGCCAGGCTCAC | | | | | | |
| | | TGACGCCCCTCCCC | | | | | | |
| | | TCCCCACAGGCCAC | | | | | | |
| | | CGTGCATGCCTCGG | | | | | | |
| | | GGAGCCCCTGGCC | | | | | | |
| | | CGCATGGAGCTCTT | | | | | | |
| | | CCTCTTCTTCACCTC | | | | | | |
| | | CCTGCTGCAGCACT | | | | | | |
| | | TCAGCTTCTCGGTG | | | | | | |
| | | CCCACTGGACAGCC | | | | | | |
| | | CCGGCCCAGCCACC | | | | | | |
| | | ATGGTGTCTTTGCTT | | | | | | |
| | | TCCTGGTGACCOCA | | | | | | |
| | | TCCCCCTATGAGCT | | | | | | |
| | | TTGTGCTGTGCCCC | | | | | | |
| | | GCTAGAATGGGGTA | | | | | | |
| | | CCTAGTCCCCAGCC | | | | | | |
| | | TGCTCCCTAGCCAG | | | | | | |

| | | |
|---|---|---|
| | | AGGCTCTAATGTAC |
| | | AATAAAGCAATGTG |
| | | GTAGTTCCAACTCG |
| | | GGTCCCCTGCTCAC |
| | | GCCCTCGTTGGGAT |
| | | CATCCTCCTCAGGG |
| | | CAACCCCACC 3′ |
| HapMap_ | NA18990, | |
| Homo WT | NA06989, | |
| | NA19143, | |
| | NA18861 | |
| HapMap_ | HG01085 | |
| Hetero | | |
| HapMap_ | N/A | |
| Homo M | | |

TABLE 33
NalaMan Intron 2

| | | | | | Conc | | |
|---|---|---|---|---|---|---|---|
| | | | | | after | | |
| | | | | | 10x | Per | Final |
| | | | | Measured | dilution | Reaction | conc. |
| Component | Name | Direction 5′-3′ | Specifications | conc. (uM) | (uM) | (uL) | (uM) |
| Master mix | SSO Advanced | NA | #1725285 | | | 12.5 | 1x |
| | Universal Probes | | | | | | |
| | Supermix | | | | | | |
| Copy Number | TaqMan Copy | NA | 60X (Size L) | NA | NA | 1.25 | NA |
| Assay | Number Assay 20X | | | | | | |
| | Hs04083572_cn | | | | | | |
| Reference | TaqMan Copy | NA | #4403328 | NA | NA | 1.25 | NA |
| Assay | Number Reference | | | | | | |
| | Assay 20X | | | | | | |
| Tris-EDTA | 1X Tris-EDTA (TE) | NA | 1st Base | NA | NA | 8 | NA |
| buffer | Buffer with | | | | | | |
| | reduced EDTA, pH | | | | | | |
| | 8.0, Biotechnology | | | | | | |
| | Grade, 1L (#CUS- | | | | | | |
| | 3022-1X1L) | | | | | | |
| Calibrator | Promega Human | NA | NA | NA | NA | | 4 ng/2 uL |
| | Genomic DNA | | | | | | |
| | (Mixed) | | | | | | |

TABLE 34
NalaMan Exon 9

| | | | | | Conc | | |
|---|---|---|---|---|---|---|---|
| | | | | | after | | |
| | | | | | 10x | Per | Final |
| | | | | Measured | dilution | Reaction | conc. |
| Component | Name | Direction 5′-3′ | Specifications | conc. (uM) | (uM) | (uL) | (uM) |
| Master mix | SSO Advanced | NA | #1725285 | | | 12.5 | 1x |
| | Universal Probes | | | | | | |
| | Supermix | | | | | | |
| Copy Number | TaqMan Copy | NA | 60X (Size L) | NA | NA | 1.25 | NA |
| Assay | Number Assay 20X | | | | | | |
| | Hs00010001_cn | | | | | | |
| Reference | TaqMan Copy | NA | #4403328 | NA | NA | 1.25 | NA |
| Assay | Number Reference | | | | | | |
| | Assay 20X | | | | | | |
| Tris-EDTA | 1X Tris-EDTA (TE) | NA | 1st Base | NA | NA | 8 | NA |
| buffer | Buffer with | | | | | | |
| | reduced EDTA, pH | | | | | | |
| | 8.0, Biotechnology | | | | | | |
| | Grade, 1L (#CUS- | | | | | | |
| | 3022-1X1L) | | | | | | |
| Calibrator | Promega Human | NA | NA | NA | NA | | 4 ng/2 uL |
| | Genomic DNA (Mixed) | | | | | | |

TABLE 35
SLCO1B1
(rs4149056)

| | | | | | Conc after | | | Amount |
|---|---|---|---|---|---|---|---|---|
| | | | | Measured | 10× | Per | Final | (nmole) per |
| | | | | conc | dilution | Reaction | conc. | rx (25ul) |
| Component | Name | Direction 5′-3′ | Specifications | (uM) | (uM) | (uL) | (uM) | |
| Master mix | SSO | NA | #1725285 | NA | NA | 12.5 | 1× | NA |
| | Advanced | | | | | | | |
| | Universal | | | | | | | |
| | Probes | | | | | | | |
| | Supermix | | | | | | | |
| Primer F | SLCO1B1_ | CTA CAT AGG TTG | 23 bp | 97.44 | 9.744 | 1.9 | 0.741 | 0.01851 |
| | 521_F | 5′ GGC TCT TAT | (Tm = 59.2) | | | | | |
| | | TT 3′ | | | | | | |
| Primer R | SLCO1B1_ | 5′ CTA TGG GAG | 20 bp | 95.04 | 9.504 | 1.9 | 0.722 | 0.01806 |
| | 521_R | TCT CCC CTA TT 3′ | (Tm = 58.4) | | | | | |
| Probe A | SLCO1B1_ | TGGGTAATATGCT/ | [6FAM]-23 bp- | 100.42 | 10.042 | 2 | 0.803 | 0.02008 |
| | 521_WT | 5′ FAM/TATGTGTTCA | [BHQ1] | | | | | |
| | | BHQ1 3′ | (Tm = 57.6) | | | | | |
| Probe B | SLCO1B1_ | HEX/ATATGCGTTC | [HEX]-22 bp- | 97.38 | 9.738 | 3 | 1.169 | 0.02922 |
| | 521_M_ | 5′ ATGGGTAATATG/I | [IBFQ] | | | | | |
| | HEX | BFQ 3′ | (Tm = 56.4) | | | | | |
| Tris-EDTA | 1× Tris- | NA | 1st Base | | | 1.70 | | NA |
| buffer | EDTA | | | | | | | |
| | (TE) | | | | | | | |
| | Buffer | | | | | | | |
| | with | | | | | | | |
| | reduced | | | | | | | |
| | EDTA, pH | | | | | | | |
| | 18.0, | | | | | | | |
| | Biotechno | | | | | | | |
| | logy | | | | | | | |
| | Grade, 1L | | | | | | | |
| | (#CUS- | | | | | | | |
| | 3022- | | | | | | | |
| | 1 × 1L) | | | | | | | |
| Template_ | 521 WT | 5′ | gblock-128 bp | | | | 100000 | 100000 |
| WT | | AAAATGAAACACT | | | | | | |
| | | CTCTTATCTACATA | | | | | | |
| | | GGTTGTTTAAAGG | | | | | | |
| | | AATCTGGGTCATA | | | | | | |
| | | CATGTGGATATAT | | | | | | |
| | | GTGTTCATGGGTA | | | | | | |
| | | ATATGCTTCGTGG | | | | | | |
| | | AATAGGGGAGACT | | | | | | |
| | | CCCATAGTACCAT | | | | | | |
| | | TGGGGCTTTC 3′ | | | | | | |
| Template_ | 521 MUT | 5′ | gblock-128 bp | | | | 100000 | 100000 |
| M | | AAAATGAAACACT | | | | | | |
| | | CTCTTATCTACATA | | | | | | |
| | | GGTTGTTTAAAGG | | | | | | |

| | | |
|---|---|---|
| | | AATCTGGGTCATA |
| | | CATGTGGATATAT |
| | | GCGTTCATGGGTA |
| | | ATATGCTTCGTGG |
| | | AATAGGGGAGACT |
| | | CCCATAGTACCAT |
| | | TGGGGCTTTC 3′ |
| HapMap_ | NA21114, | |
| Homo WT | HG00111 | |
| HapMap_ | HG00358, | |
| Hetero | HG00524 | |
| HapMap_ | NA18608, | |
| Homo M | NA19000, | |
| | NA10847 | |

TABLE 36
CYP2C9*2
(rs1799853)

| | | | | | Conc | | | |
|---|---|---|---|---|---|---|---|---|
| | | | | | after | | | Amount |
| | | | | Measured | 10× | Per | Final | (nmole) |
| | | | | conc. | dilution | Reaction | conc. | per rx |
| Component | Name | Direction 5′-3′ | Specifications | (uM) | (uM) | (uL) | (uM) | (25 ul) |
| Master mix | SSO | NA | #1725285 | NA | NA | 12.5 | 1× | NA |
| | Advanced | | | | | | | |
| | Universal | | | | | | | |
| | Probes | | | | | | | |
| | Supermix | | | | | | | |
| Primer F | CYP2C9*2- | 5′ GC GTT TCT | 18 bp | 93.05 | 9.305 | 0.5 | 0.186 | 0.00465 |
| | F7a | CCC TCA TGA C 3′ | (Tm = 56.3) | | | | | |
| Primer R | CYP2C9*2_ | 5′ | 20 bp | 91.26 | 9.126 | 0.5 | 0.183 | 0.00456 |
| | R1_Sa | GGTCAGTGATATG | (Tm = 58.4) | | | | | |
| | | GAGTAGG 3′ | | | | | | |
| Probe A | CYP2C9*2- | FAM/CATTGAGGAC | [6FAM]-22 bp- | 93.99 | 9.399 | 1.5 | 0.564 | 0.01410 |
| | P1a | 5′ CGTGTTCAAGAG/ | [BHQ1] | | | | | |
| | | BHQ1 3′ | (Tm = 62.1) | | | | | |
| Probe B | CYP2C9*2- | 5′ | [HEX]-22 | 94.32 | 9.432 | 1 | 0.377 | 0.00943 |
| | P1am | HEX/CATTGAGGAC | bp-[BHQ1] | | | | | |
| | | TGTGTTCAAGAG/ | (Tm = 60.1) | | | | | |
| | | BHQ1 3′ | | | | | | |
| Tris-EDTA | 1× Tris- | NA | 1 st Base | | | 7.00 | | NA |
| buffer | EDTA (TE) | | | | | | | |
| | Buffer with | | | | | | | |
| | reduced | | | | | | | |
| | EDTA, pH | | | | | | | |
| | 8.0, | | | | | | | |
| | Biotech- | | | | | | | |
| | nology Grade, | | | | | | | |
| | 1L (#CUS- | | | | | | | |
| | 3022-1 × 1L) | | | | | | | |
| Template_ | CYP2C9_ | TTTCAGCATCTGT | gblock-500 bp | | | | 100000 | 100000 |
| WT | WT_C9*2 | CTTGGGGATGGG | | | | | | |
| | | GAGGATGGAAAAC | | | | | | |
| | | AGAGACTTACAGA | | | | | | |
| | | GCTCCTCGGGCAG | | | | | | |
| | | AGCTTGGCCCATC | | | | | | |
| | | CACATGGCTGCCC | | | | | | |
| | | AGTGTCAGCTTCC | | | | | | |
| | | TCTTTCTTGCCTG | | | | | | |
| | | GGATCTCCCTCCT | | | | | | |
| | | AGTTTCGTTTCTCT | | | | | | |
| | | TCCTGTTAGGAAT | | | | | | |
| | | TGTTTTCAGCAAT | | | | | | |
| | | GGAAAGAAATGGA | | | | | | |
| | | AGGAGATCCGGC | | | | | | |
| | | GTTTCTCCCTCAT | | | | | | |
| | | GACGCTGCGGAAT | | | | | | |
| | | TTTGGGATGGGGA | | | | | | |
| | | AGAGGAGCATTGA | | | | | | |
| | | GGACCGTGTTCAA | | | | | | |
| | | GAGGAAGCCCGCT | | | | | | |
| | | GCCTTGTGGAGGA | | | | | | |
| | | GTTGAGAAAAACC | | | | | | |
| | | AAGGGTGGGTGAC | | | | | | |
| | | CCTACTCCATATC | | | | | | |
| | | ACTGACCTTACTG | | | | | | |
| | | GACTACTATCTTCT | | | | | | |
| | | CTACTGACATTCTT | | | | | | |
| | | GGAAACATTTCAG | | | | | | |
| | | GGGTGGCCATATC | | | | | | |
| | | TTTCATTATGAGTC | | | | | | |
| | | CTGGTTGTTAGCT | | | | | | |
| | | CATGTGAAGCGGG | | | | | | |
| | | GGTTTGAAGCTGA | | | | | | |
| | | GAGCCAAGGGAAT | | | | | | |
| | | TTGCACATATTTGT | | | | | | |
| | | GCTGTGTGTGTAC | | | | | | |
| | | AGGCATGATTGTG | | | | | | |
| | | CGT | | | | | | |
| Template_ | CYP2C9*2_ | TTTCAGCATCTGT | gblock-500 bp | | | | 100000 | 100000 |
| M | MT | CTTGGGGATGGG | | | | | | |
| | | GAGGATGGAAAAC | | | | | | |
| | | AGAGACTTACAGA | | | | | | |
| | | GCTCCTCGGGCAG | | | | | | |
| | | AGCTTGGCCCATC | | | | | | |
| | | CACATGGCTGCCC | | | | | | |
| | | AGTGTCAGCTTCC | | | | | | |
| | | TCTTTCTTGCCTG | | | | | | |
| | | GGATCTCCCTCCT | | | | | | |
| | | AGTTTCGTTTCTCT | | | | | | |
| | | TCCTGTTAGGAAT | | | | | | |
| | | TGTTTTCAGCAAT | | | | | | |
| | | GGAAAGAAATGGA | | | | | | |
| | | AGGAGATCCGGC | | | | | | |
| | | GTTTCTCCCTCAT | | | | | | |
| | | GACGCTGCGGAAT | | | | | | |
| | | TTTGGGATGGGGA | | | | | | |
| | | AGAGGAGCATTGA | | | | | | |
| | | GGACTGTGTTCAA | | | | | | |
| | | GAGGAAGCCCGCT | | | | | | |
| | | GCCTTGTGGAGGA | | | | | | |
| | | GTTGAGAAAAACC | | | | | | |
| | | AAGGGTGGGTGAC | | | | | | |
| | | CCTACTCCATATC | | | | | | |
| | | ACTGACCTTACTG | | | | | | |
| | | GACTACTATCTTCT | | | | | | |
| | | CTACTGACATTCTT | | | | | | |
| | | GGAAACATTTCAG | | | | | | |
| | | GGGTGGCCATATC | | | | | | |
| | | TTTCATTATGAGTC | | | | | | |
| | | CTGGTTGTTAGCT | | | | | | |
| | | CATGTGAAGCGGG | | | | | | |
| | | GGTTTGAAGCTGA | | | | | | |
| | | GAGCCAAGGGAAT | | | | | | |
| | | TTGCACATATTTGT | | | | | | |
| | | GCTGTGTGTGTAC | | | | | | |
| | | AGGCATGATTGTG | | | | | | |
| | | CGT | | | | | | |
| HapMap_ | NA19143 | | | | | | | |
| Homo WT | | | | | | | | |

| | |
|---|---|
| HapMap_ | HG00358 |
| Hetero | |
| HapMap_ | NA06989 |
| Homo M | |

TABLE 37
CYP2C9*3
(rs1057910)

| | | | | | Conc | | | |
|---|---|---|---|---|---|---|---|---|
| | | | | | after | | | Amount |
| | | | | Measured | 10× | Per | Final | (nmole) |
| | | | | conc. | dilution | Reaction | conc. | per rx |
| Component | Name | Direction 5′-3′ | Specifications | (uM) | (uM) | (uL) | (uM) | (25 ul) |
| Master mix | SSO | NA | #1725285 | NA | NA | 12.5 | 1× | NA |
| | Advanced | | | | | | | |
| | Universal | | | | | | | |
| | Probes | | | | | | | |
| | Supermix | | | | | | | |
| Primer F | CYP2C9*3- | 5′ | 18 bp | 95.53 | 9.553 | 0.5 | 0.191 | 0.00478 |
| | F2 | CTGCATGCAAGAC | (Tm = 56.3) | | | | | |
| | | AGGAG 3′ | | | | | | |
| Primer R | CYP2C9*3- | 5′ | 23 bp | 91.81 | 9.181 | 0.5 | 0.184 | 0.00459 |
| | R2 | CCTTGGGAATGAG | (Tm = 60.9) | | | | | |
| | | ATAGTTTCTG 3′ | | | | | | |
| Probe A | CYP2C9*3- | 5′ 6- | [6FAM]-21 bp- | 93.29 | 9.329 | 1.5 | 0.560 | 0.01399 |
| | P4a | FAM/CGAGGTCCA | [BHQ1] | | | | | |
| | | GAGATACATTGA/ | (Tm = 59.5) | | | | | |
| | | BHQ1 3′ | | | | | | |
| Probe B | CYP2C9*3- | 5′ | [HEX]-21 bp- | 101.02 | 10.102 | 1.25 | 0.505 | 0.01263 |
| | MT-P4a- | HEX/CGAGGTCCA | [IBFQ] | | | | | |
| | HEX | GAGATACCTTGA/ | (Tm = 61.2) | | | | | |
| | | IBFQ 3′ | | | | | | |
| Tris-EDTA | 1× Tris- | NA | 1^{st} Base | | | 6.75 | | NA |
| buffer | EDTA (TE) | | | | | | | |
| | Buffer with | | | | | | | |
| | reduced | | | | | | | |
| | EDTA, pH | | | | | | | |
| | 8.0, | | | | | | | |
| | Biotech- | | | | | | | |
| | nology Grade, | | | | | | | |
| | 1L (#CUS- | | | | | | | |
| | 3022-1 × 1L) | | | | | | | |
| Template | CYP2C9*3- | 5′ CCTGATGAAAATG | gblock-500 bp | | | | 100000 | 100000 |
| WT | WT | GAGAAGGAAAAGC | | | | | | |
| | | ACAACCAACCATC | | | | | | |
| | | TGAATTTACTATTG | | | | | | |
| | | AAAGCTTGGAAAA | | | | | | |
| | | CACTGCAGTTGAC | | | | | | |
| | | TTGTTTGGAGCTG | | | | | | |
| | | GGACAGAGACGAC | | | | | | |
| | | AAGCACAACCCTG | | | | | | |
| | | TTCTCCTGCTGAA | | | | | | |
| | | GCACCCAGAGGTC | | | | | | |
| | | ACAGCTAAAGTCC | | | | | | |
| | | AGGAAGAGATTGA | | | | | | |
| | | ACGTGTGATTGGC | | | | | | |
| | | AGAAACCGGAGCC | | | | | | |
| | | CCTGCATGCAAGA | | | | | | |
| | | CAGGAGCCACATG | | | | | | |
| | | CCCTACACAGATG | | | | | | |
| | | CTGTGGTGCACGA | | | | | | |
| | | GGTCCAGAGATAC | | | | | | |
| | | ATTGACCTTCTCC | | | | | | |
| | | CCACCAGCCTGCC | | | | | | |
| | | CCATGCAGTGACC | | | | | | |
| | | TGTGACATTAAATT | | | | | | |
| | | CAGAAACTATCTC | | | | | | |
| | | ATTCCCAAGGGCA | | | | | | |
| | | CAACCATATTAATT | | | | | | |
| | | TCCCTGACTTCTG | | | | | | |
| | | TGCTACATGACAA | | | | | | |
| | | CAAAGAATTTCCC | | | | | | |
| | | AACCCAGAGATGT | | | | | | |
| | | TTGACCCTCATCA | | | | | | |
| | | CTTTCTGGATGAA | | | | | | |
| | | GGTGGCAATTTTA | | | | | | |
| | | AGAAAAGTAAATA | | | | | | |
| | | CTTCATGCCTTTCT | | | | | | |
| | | CAGCAGGAAAACG | | | | | | |
| | | GA 3′ | | | | | | |
| Template_ | CYP2C9*3- | 5′ CCTGATGAAAATG | gblock-500bp | | | | 10000 | 100000 |
| M | MT | GAGAAGGAAAAGC | | | | | 0 | |
| | | ACAACCAACCATC | | | | | | |
| | | TGAATTTACTATTG | | | | | | |
| | | AAAGCTTGGAAAA | | | | | | |
| | | CACTGCAGTTGAC | | | | | | |
| | | TTGTTTGGAGCTG | | | | | | |
| | | GGACAGAGACGAC | | | | | | |
| | | AAGCACAACCCTG | | | | | | |
| | | AGATATGCTCTCC | | | | | | |
| | | TTCTCCTGCTGAA | | | | | | |
| | | GCACCCAGAGGTC | | | | | | |
| | | ACAGCTAAAGTCC | | | | | | |
| | | AGGAAGAGATTGA | | | | | | |
| | | ACGTGTGATTGGC | | | | | | |
| | | AGAAACCGGAGCC | | | | | | |
| | | CCTGCATGCAAGA | | | | | | |
| | | CAGGAGCCACATG | | | | | | |
| | | CTGTGGTGCACGA | | | | | | |
| | | GGTCCAGAGATAC | | | | | | |
| | | CTTGACCTTCTCC | | | | | | |
| | | CCACCAGCCTGCC | | | | | | |
| | | CCATGCAGTGACC | | | | | | |
| | | TGTGACATTAAATT | | | | | | |
| | | CAGAAACTATCTC | | | | | | |
| | | ATTCCCAAGGGCA | | | | | | |
| | | CAACCATATTAATT | | | | | | |
| | | TCCCTGACTTCTG | | | | | | |
| | | TGCTACATGACAA | | | | | | |
| | | CAAAGAATTTCCC | | | | | | |
| | | AACCCAGAGATGT | | | | | | |
| | | TTGACCCTCATCA | | | | | | |
| | | CTTTCTGGATGAA | | | | | | |
| | | GGTGGCAATTTTA | | | | | | |
| | | AGAAAAGTAAATA | | | | | | |
| | | CTTCATGCCTTTCT | | | | | | |
| | | CAGCAGGAAAACG | | | | | | |
| | | GA 3′ | | | | | | |
| HapMap_ | NA18861, | | | | | | | |
| Homo WT | NA06989, | | | | | | | |
| | NA19143 | | | | | | | |
| HapMap_ | NA12005, | | | | | | | |
| Hetero | NA18959 | | | | | | | |
| HapMap_ | NA21114 | | | | | | | |
| Homo M | | | | | | | | |

TABLE 38
CYP2C19*2
(rs4244285)

| | | | | | Conc | | | |
|---|---|---|---|---|---|---|---|---|
| | | | | | after | | | Amount |
| | | | | Measured | 10× | Per | Final | (nmole) |
| | | | | conc. | dilution | Reaction | conc | per rx |
| Component | Name | Direction 5′-3′ | Specifications | (uM) | (uM) | (uL) | (uM) | (25 ul) |
| Master mix | SSO | NA | #1725285 | NA | NA | 12.5 | 1× | NA |
| | Advanced | | | | | | | |
| | Universal | | | | | | | |
| | Probes | | | | | | | |
| | Supermix | | | | | | | |
| Primer F | CYP2C19*2- | 5′ | 19 bp | 95.62 | 9.562 | 0.5 | 0.191 | 0.00478 |
| | F2 | CACCCCCTGGATC | (Tm = 59.5) | | | | | |
| | | CAGATA 3′ | | | | | | |
| Primer R | CYP2C19*2- | 5′ | 22 bp | 96.17 | 9.617 | 0.5 | 0.192 | 0.00481 |
| | R1 | TCTCCAAAATATCA | (Tm = 54.7) | | | | | |
| | | CTTTCCAT 3′ | | | | | | |
| Probe A | CYP2C19*2- | 5′ 6- | [6FAM]-22 bp- | 99.35 | 9.935 | 0.25 | 0.099 | 0.00248 |
| | P2 | FAM/TCATTGATTA | [BHQ1] | | | | | |
| | | TTTCCCGGGAAC/ | (Tm = 58.4) | | | | | |
| | | BHQ1 3′ | | | | | | |
| Probe B | CYP2C19*2- | 5′ | [HEX]-22 bp- | 93.14 | 9.314 | 0.25 | 0.093 | 0.00233 |
| | MT-P2-HEX | HEX/TCATTGATTA | [IBFQ] | | | | | |
| | | TTTCCCAGGAAC/ | (Tm = 56.4) | | | | | |
| | | IBFQ 3′ | | | | | | |
| Tris-EDTA | 1× Tris- | NA | 1^{st} Base | | | 9.00 | | NA |
| buffer | EDTA (TE) | | | | | | | |
| | Buffer with | | | | | | | |
| | reduced | | | | | | | |
| | EDTA, pH | | | | | | | |
| | 8.0, | | | | | | | |
| | Bio- | | | | | | | |
| | technology | | | | | | | |
| | Grade, | | | | | | | |
| | 1L (#CUS- | | | | | | | |
| | 3022-1 × 1L) | | | | | | | |
| Template_ | CYP2C19*2- | 5′ CAGAGGATTTGGA | gblock-500 bp | | | | 100000 | 100000 |
| WT | WT | ATCGTTTTCAGCA | | | | | | |
| | | ATGGAAAGAGATG | | | | | | |
| | | GAAGGAGATCCGG | | | | | | |
| | | CGTTTCTCCCTCA | | | | | | |
| | | TGACGCTGCGGAA | | | | | | |
| | | TTTTGGGATGGGG | | | | | | |
| | | AAGAGGAGCATTG | | | | | | |
| | | AGGACCGTGTTCA | | | | | | |
| | | AGAGGAAGCCCG | | | | | | |
| | | CTGCCTTGTGGAG | | | | | | |
| | | GAGTTGAGAAAAA | | | | | | |
| | | CTGTGATCCCACT | | | | | | |
| | | TTCATCCTGGGCT | | | | | | |
| | | GTGCTCCCTGCAA | | | | | | |
| | | TGTGATCTGCTCC | | | | | | |
| | | ATTATTTTCCAGAA | | | | | | |
| | | ACGTTTCGATTATA | | | | | | |
| | | AAGATCAGCAATT | | | | | | |
| | | TCTTAACTTGATG | | | | | | |
| | | GAAAAATTGAATG | | | | | | |
| | | AAAACATCAGGAT | | | | | | |
| | | TGTAAGCACCCCC | | | | | | |
| | | TGGATCCAGATAT | | | | | | |
| | | GCAATAATTTTCCC | | | | | | |
| | | ACTATCATTGATTA | | | | | | |
| | | TTTCCCGGGAACC | | | | | | |
| | | CATAACAAATTACT | | | | | | |
| | | TAAAAACCTTGCTT | | | | | | |
| | | TTATGGAAAGTGA | | | | | | |
| | | TATTTTGGAGAAA | | | | | | |
| | | GTAAAAGAACACC | | | | | | |
| | | AAGAATCGATGGA | | | | | | |
| | | CATCAACAACCCT | | | | | | |
| | | CGGGACTTTATTG | | | | | | |
| | | ATTGCTTCCTGAT | | | | | | |
| | | CAAAATGGAGAAG | | | | | | |
| | | G 3′ | | | | | | |
| Template_ | CYP2C19*2- | 5′ CAGAGGATTTGGA | gblock-500 bp | | | | 100000 | 100000 |
| M | MT | ATCGTTTTCAGCA | | | | | | |
| | | ATGGAAAGAGATG | | | | | | |
| | | GAAGGAGATCCGG | | | | | | |
| | | CGTTTCTCCCTCA | | | | | | |
| | | TGACGCTGCGGAA | | | | | | |
| | | TTTTGGGATGGGG | | | | | | |
| | | AAGAGGAGCATTG | | | | | | |
| | | AGGACCGTGTTCA | | | | | | |
| | | AGAGGAAGCCCG | | | | | | |
| | | CTGCCTTGTGGAG | | | | | | |
| | | GAGTTGAGAAAAA | | | | | | |
| | | CCAAGGCTTCACC | | | | | | |
| | | CTGTGATCCCACT | | | | | | |
| | | TTCATCCTGGGCT | | | | | | |
| | | GTGCTCCCTGCAA | | | | | | |
| | | TGTGATCTGCTCC | | | | | | |
| | | ATTATTTTCCAGAA | | | | | | |
| | | AAGATCAGCAATT | | | | | | |
| | | TCTTAACTTGATG | | | | | | |
| | | GAAAAATTGAATG | | | | | | |
| | | AAAACATCAGGAT | | | | | | |
| | | TGTAAGCACCCCC | | | | | | |
| | | TGAATCCAGATAT | | | | | | |
| | | GCAATAATTTTCCC | | | | | | |
| | | ACTATCATTGATTA | | | | | | |
| | | TTTCCCAGGAACC | | | | | | |
| | | CATAACAAATTACT | | | | | | |
| | | TAAAAACCTTGCTT | | | | | | |
| | | TTATGGAAAGTGA | | | | | | |
| | | TATTTTGGAGAAA | | | | | | |
| | | GTAAAAGAACACC | | | | | | |
| | | AAGAATCGATGGA | | | | | | |
| | | CATCAACAACCCT | | | | | | |
| | | CGGGACTTTATTG | | | | | | |
| | | ATTGCTTCCTGAT | | | | | | |
| | | CAAAATGGAGAAG | | | | | | |
| | | G 3′ | | | | | | |
| HapMap_ | HG02684, | | | | | | | |
| Homo WT | HG01398, | | | | | | | |
| | NA06989, | | | | | | | |
| | NA19143 | | | | | | | |
| HapMap_ | NA19201 | | | | | | | |
| Hetero | | | | | | | | |
| HapMap_ | NA18961 | | | | | | | |
| Homo M | | | | | | | | |

TABLE 39
CYP2C19*3
(rs4986893)

| | | | | | Conc | | | |
|---|---|---|---|---|---|---|---|---|
| | | | | | after | | | Amount |
| | | | | Measured | 10× | Per | Final | (nmole) |
| | | | | conc | dilution | Reaction | conc. | per rx |
| Component | Name | Direction 5′-3′ | Specifications | (uM) | (uM) | (uL) | (uM) | (25 ul) |
| Master mix | SSO | NA | #1725285 | NA | NA | 12.5 | 1× | NA |
| | Advanced | | | | | | | |
| | Universal | | | | | | | |
| | Probes | | | | | | | |
| | Supermix | | | | | | | |
| Primer F | CYP2C19*3_ | 5′ CCA TTA TTT | 24 bp | 92.74 | 9.274 | 0.5 | 0.185 | 0.0046 |
| | rF1 | TCC AGA AAC GTT | (Tm = 60.3) | | | | | 4 |
| | | TCG 3′ | | | | | | |
| Primer R | CYP2C19*3_ | 5′ GGA TTT CCC | 23 bp | 92.25 | 9.225 | 0.5 | 0.185 | 0.0046 |
| | rR5 | AGA AAA AAA GAC | (Tm = 59.2) | | | | | 1 |
| | | TG 3′ | | | | | | |
| Probe A | CYP2C19*3_ | 5′ FAM/TA AGC | [6FAM]-21 bp- | 103.64 | 10.364 | 2 | 0.829 | 0.0207 |
| | rP1 | ACC CCC TGG ATC | [BHQ1] | | | | | 3 |
| | | CAG G/BHQ1 3′ | (Tm = 65.3) | | | | | |
| Probe B | CYP2C19*3_ | 5′ HEX/TA AGC | [HEX]-21 bp- | 102.48 | 10.248 | 3 | 1.230 | 0.0307 |
| | rP1aM | ACC CCC TGA ATC | [IBFQ] | | | | | 4 |
| | | CAG G/IBFQ 3′ | (Tm = 63.2) | | | | | |
| Tris-EDTA | 1× Tris- | NA | 1^{st} Base | | | 4.50 | | NA |
| buffer | EDTA (TE) | | | | | | | |
| | Buffer with | | | | | | | |
| | reduced | | | | | | | |
| | EDTA, pH | | | | | | | |
| | 8.0, | | | | | | | |
| | Bio- | | | | | | | |
| | technology | | | | | | | |
| | Grade, | | | | | | | |
| | 1L (#CUS- | | | | | | | |
| | 3022-1 × 1L) | | | | | | | |
| Template_ | CYP2C19_ | TTATATCTAATGTT | gblock-452 bp | | | | 100000 | 100000 |
| WT | WT_C19*3 | TACTCATATTTTAA | | | | | | |
| | | AATTGTTTCCAATC | | | | | | |
| | | ATTTAGCTTCACC | | | | | | |
| | | CTGTGATCCCACT | | | | | | |
| | | TTCATCCTGGGCT | | | | | | |
| | | GTGCTCCCTGCAA | | | | | | |
| | | TGTGATCTGCTCC | | | | | | |
| | | ATTATTTTCCAGAA | | | | | | |
| | | ACGTTTCGATTATA | | | | | | |
| | | AAGATCAGCAATT | | | | | | |
| | | TCTTAACTTGATG | | | | | | |
| | | GAAAAATTGAATG | | | | | | |
| | | AAAACATCAGGAT | | | | | | |
| | | TGTAAGCACCCCC | | | | | | |
| | | TGGATCCAGGTAA | | | | | | |
| | | GGCCAAGTTTTTT | | | | | | |
| | | GCTTCCTGAGAAA | | | | | | |
| | | CCACTTACAGTCT | | | | | | |
| | | TTTTTTCTGGGAAA | | | | | | |
| | | TCCAAAATTCTATA | | | | | | |
| | | TTGACCAAGCCCT | | | | | | |
| | | GAAGTACATTTTTG | | | | | | |
| | | AATACTACAGTCTT | | | | | | |
| | | GCCTAGACAGCCA | | | | | | |
| | | TGGGGTGAATATC | | | | | | |
| | | TGGAAAAGATGGC | | | | | | |
| | | AAAGTTCTTTATTT | | | | | | |
| | | TATGCACAGGAAA | | | | | | |
| | | TGAATATCCCAATA | | | | | | |
| | | TAGATCAGGCTTC | | | | | | |
| | | TAAGCCCATTAGC | | | | | | |
| | | TCCCTGATCAGTG | | | | | | |
| | | TTTTTTCCACTA | | | | | | |
| Template_ | CYP2C19_ | TATATCTAATGTTT | gblock-451 bp | | | | 100000 | 100000 |
| M | MT | ACTCATATTTTAAA | | | | | | |
| | | ATTGTTTCCAATCA | | | | | | |
| | | TTTAGCTTCACCCT | | | | | | |
| | | GTGATCCCACTTT | | | | | | |
| | | CATCCTGGGCTGT | | | | | | |
| | | GCTCCCTGCAATG | | | | | | |
| | | TGATCTGCTCCAT | | | | | | |
| | | TATTTTCCAGAAAC | | | | | | |
| | | GTTTCGATTATAAA | | | | | | |
| | | GATCAGCAATTTC | | | | | | |
| | | TTAACTTGATGGA | | | | | | |
| | | AAAATTGAATGAAA | | | | | | |
| | | ACATCAGGATTGT | | | | | | |
| | | AAGCACCCCCTGA | | | | | | |
| | | ATCCAGGTAAGGC | | | | | | |
| | | CAAGTTTTTTGCTT | | | | | | |
| | | CCTGAGAAACCAC | | | | | | |
| | | TTACAGTCTTTTTT | | | | | | |
| | | TCTGGGAAATCCA | | | | | | |
| | | AAATTCTATATTGA | | | | | | |
| | | CCAAGCCCTGAAG | | | | | | |
| | | TACATTTTTGAATA | | | | | | |
| | | CTACAGTCTTGCC | | | | | | |
| | | TAGACAGCCATGG | | | | | | |
| | | GGTGAATATCTGG | | | | | | |
| | | AAAAGATGGCAAA | | | | | | |
| | | GTTCTTTATTTTAT | | | | | | |
| | | GCACAGGAAATGA | | | | | | |
| | | ATATCCCAATATAG | | | | | | |
| | | ATCAGGCTTCTAA | | | | | | |
| | | GCCCATTAGCTCC | | | | | | |
| | | CTGATCAGTGTTTT | | | | | | |
| | | TTCCACTA | | | | | | |
| HapMap_ | NA12762, | | | | | | | |
| Homo WT | NA06989, | | | | | | | |
| | NA19143 | | | | | | | |
| HapMap_ | NA18564, | | | | | | | |
| Hetero | NA18608 | | | | | | | |
| HapMap_H | NA18971 | | | | | | | |
| omo M | | | | | | | | |

Table 39

TABLE 40
CYP2C19*17
(rs12248560)

| | | | | | Conc | | | |
|---|---|---|---|---|---|---|---|---|
| | | | | | after | | | Amount |
| | | | | Measured | 10× | Per | Final | (nmole) |
| | | | | conc. | dilution | Reaction | conc. | per rx |
| Component | Name | Direction 5′-3′ | Specifications | (uM) | (uM) | (uL) | (uM) | (25 ul) |
| Master mix | SSO | NA | #1725285 | NA | NA | 12.5 | 1× | NA |
| | Advanced | | | | | | | |
| | Universal | | | | | | | |
| | Probes | | | | | | | |
| | Supermix | | | | | | | |
| Primer F | CYP2C19*17- | 5′ | 23 bp | 93.27 | 9.327 | 0.5 | 0.187 | 0.0046 |
| | F7 | AACAAAGTTTTAG | (Tm = 53.9) | | | | | 6 |
| | | CAAACGATTT 3′ | | | | | | |
| Primer R | CYP2C19*17- | 5′ | 17 bp | 92.23 | 9.223 | 0.1 | 0.037 | 0.0009 |
| | R3 | ATGCCCATCGTGG | (Tm = 57.3) | | | | | 2 |
| | | CGCA 3′ | | | | | | |
| Probe A | CYP2C19*17- | 5′ 6-FAM/ | [6FAM]-20 bp- | 99.52 | 9.952 | 2.5 | 0.995 | 0.0248 |
| | P2a | TCTTCTGTTC | [BHQ1] | | | | | 8 |
| | | TCAAAGCATC/BHQ1 | (Tm = 54.3) | | | | | |
| | | 3′ | | | | | | |
| Probe B | CYP2C19*17- | 5′ HEX/TGTCTTCT | [HEX]-20 bp- | 107.13 | 10.713 | 0.5 | 0.214 | 0.0053 |
| | MT- | GTTCTCAAAGTA/ | [IBFQ] | | | | | 6 |
| | P1a_HEX | IBFQ 3′ | (Tm = 52.3) | | | | | |
| Tris-EDTA | 1× Tris- | NA | 1^{st} Base | | | 6.90 | | NA |
| buffer | EDTA (TE) | | | | | | | |
| | Buffer with | | | | | | | |
| | reduced | | | | | | | |
| | EDTA, pH | | | | | | | |
| | 8.0, | | | | | | | |
| | Bio- | | | | | | | |
| | technology | | | | | | | |
| | Grade, | | | | | | | |
| | 1L (#CUS- | | | | | | | |
| | 3022-1 × 1L) | | | | | | | |
| Template_ | CYP2C19*17- | GCCTGTTTTATGA | gblock-219 bp | | | | 100000 | 100000 |
| WT | WT | ACAGGATGAATGT | | | | | | |
| | | GGTATATATTCAG | | | | | | |
| | | AATAACTAATGTTT | | | | | | |
| | | GGAAGTTGTTTTG | | | | | | |
| | | TTTTGCTAAAACAA | | | | | | |
| | | AGTTTTAGCAAAC | | | | | | |
| | | GATTTTTTTTTTCA | | | | | | |
| | | AATTTGTGTCTTCT | | | | | | |
| | | GTTCTCAAAGCAT | | | | | | |
| | | CTCTGATGTAAGA | | | | | | |
| | | GATAATGCGCCAC | | | | | | |
| | | GATGGGCATCAGA | | | | | | |
| | | AGACCTCAGCTCA | | | | | | |
| | | AATCCCAGTTCTG | | | | | | |
| | | CCAGCTATGAGCT | | | | | | |
| | | GTGTGGC | | | | | | |
| Template_ | CYP2C19*17- | TTTGTTTTGCTAAA | gblock-369 bp | | | | 100000 | 100000 |
| M | MT | CTGAGCATTTCCC | | | | | | |
| | | CTCTGCAGTGATG | | | | | | |
| | | GAGAAGGGAGAAC | | | | | | |
| | | TCTTATTTTTTCTC | | | | | | |
| | | ATGAGCATCTCTG | | | | | | |
| | | GGGCTGTTTTCCT | | | | | | |
| | | TAGATAAATAAGT | | | | | | |
| | | GGTTCTATTTAATG | | | | | | |
| | | TGAAGCCTGTTTT | | | | | | |
| | | ATGAACAGGATGA | | | | | | |
| | | ATGTGGTATATATT | | | | | | |
| | | CAGAATAACTAAT | | | | | | |
| | | GTTTGGAAGTTGT | | | | | | |
| | | ACAAAGTTTTAGC | | | | | | |
| | | AAACGATTTTTTTT | | | | | | |
| | | TTCAAATTTGTGTC | | | | | | |
| | | TTCTGTTCTCAAAG | | | | | | |
| | | TATCTCTGATGTAA | | | | | | |
| | | GAGATAATGCGCC | | | | | | |
| | | ACGATGGGCATCA | | | | | | |
| | | GAAGACCTCAGCT | | | | | | |
| | | CAAATCCCAGTTC | | | | | | |
| | | TGCCAGCTATGAG | | | | | | |
| | | CTGTGTGGCACCA | | | | | | |
| | | ACAGGTGTCCTGT | | | | | | |
| | | TCTCCCAGGGTCT | | | | | | |
| | | CCCTTTTCCC | | | | | | |
| HapMap_ | NA12003 | | | | | | | |
| Homo WT | | | | | | | | |
| HapMap_ | NA12872 | | | | | | | |
| Hetero | | | | | | | | |
| HapMap_ | NA19098, | | | | | | | |
| Homo M | NA19153, | | | | | | | |
| | NA12812, | | | | | | | |
| | NA19346 | | | | | | | |

### REFERENCES INCORPORATED BY REFERENCE

Kothary, A. S., Mahendra, C., Tan, M., Min Tan, E J., Hong Yi, J. P., Gabriella, Hui Jocelyn, T. X., Haruman, J. S., Tan, Z., Lee, C. K., Lezhava, A., Yan, B., & Irwanto, A. (2021). Validation of a multi-gene qPCR-based pharmacogenomics panel across major ethnic groups in Singapore and Indonesia. Pharmacogenomics, 22(16), 1041-1056. https://doi.org/10.2217/pgs-2021-0071
Maggadani, B. P., Junusmin, K. I., Sani, L. L., Mahendra, C., Amelia, M., Gabriella, Irwanto, A., Harmita, Harahap, Y., &amp; Haryono, S. J, (2021). CYP2D6 genotyping for personalized therapy of tamoxifen in Indonesian women with ER+ breast cancer, https://doi.org/10.1101/2021.06.25.21259564

1. A method of assessing or evaluating a subject's likelihood of developing an adverse reaction in response to an administration of a therapeutic agent, or a method of assessing or evaluating a therapeutic agent's efficacy on a subject, the method comprising determining in a single real-time polymerase chain reaction run the presence of a variant in a set of genes consisting of CYP2D6, CYP2C9, CYP2C19 and SLCO1B1 in a sample obtained from the subject, wherein the presence of a variant on any one of the genes in the set of genes is indicative of a risk of an adverse reaction and/or change in efficacy to the therapeutic agent.

2. The method according to claim 1, wherein the presence of a variant is determined by providing a plurality of primer pairs and probes for amplifying a nucleic acid in the sample, wherein each primer pair amplifies a region of the nucleic acid associated with the genes or its variant, and detecting the presence or absence of a polymerase chain reaction product is indicative of the variant.

3. The method according to claim 1 or 2, wherein the variant of the gene is any variant selected from the group consisting of rs1065852, rs5030655, rs3892097, rs35742686, rs16947, rs28371725, rs1135840, rs769258, rs5030865, rs5030656, rs59421388, rs267608319, exon 9 conversion (*36), deletion (*5), rs1799853, rs1057910, rs4244285, rs4986893, rs12248560 and rs4149056.

4. The method according to any one of claim 2 or 3, wherein the plurality of primer pairs and probes is any one selected from the list in Tables 3 and 4.

5. The method according to any one of claims 2 to 4, wherein the plurality of primer pairs comprises at least one primer pair for amplifying a conserved area of the gene.

6. The method according to any one of the preceding claims, wherein the variant is a copy number variation and wherein the step of determining the presence of the copy number variation further comprising an RNaseP as a housekeeping gene.

7. The method according to claim 6, wherein the step of determining the presence of the copy number variation further comprising providing a control having a human genomic DNA to determine the subject's CYP2D6 gene copy number variations.

8. The method according to any one of claims 2 to 7, wherein the probes for targeting non-variant genes are tagged with a FAM fluorophore at the 5′ end, and the probes for targeting variant genes are tagged with HEX or Cy5 fluorophore at the 5′ end.

9. The method according to claim 6, wherein the variant is a copy number variation of CYP2D6 and wherein the probes for targeting the copy number variation of CYP2D6 are tagged with a FAM fluorophore at the 5′, and the probes for targeting the housekeeping gene are tagged with a VIC fluorophore at the 5′ end.

10. The method according to claim 9, wherein the probes have a 3′ modification of either a BHQ1 quencher, an IBFQ quencher, or an IBRQ quencher.

11. The method according to any one of claims 8 to 10, wherein the ratio between primer pairs and FAM, HEX, Cy5 probes are asymmetric.

12. The method according to any one of the preceding claims, wherein the therapeutic agent is any one selected from the list in Table 2.

13. The method according to any one of the preceding claims, wherein the single real-time polymerase chain reaction run comprises 50 cycles of denaturation and annealing/extension, said denaturation is carried out at about 95° C. for about 15 seconds and said annealing/extension is carried out at about 60° C. for about 60 seconds.

14. A kit comprising means for assessing or evaluating a subject's likelihood of developing an adverse reaction in response to an administration of a therapeutic agent, or for assessing or evaluating a therapeutic agent's efficacy on a subject by determining in a single real-time polymerase chain reaction run the presence of a variant in a set of genes consisting of CYP2D6, CYP2C9, CYP2C19 and SLCO1B1 in a sample obtained from the subject, wherein the presence of a variant on any one of the genes in the set of genes is indicative of a risk of an adverse reaction and/or change in efficacy to the therapeutic agent.

15. The kit according to claim 14, wherein the means comprising a plurality of primer pairs and probes selected from the list in Tables 3 and 4.
